(19) United States
(12) Patent Application Publication
(10) Pub. No.: US 2025/0333381 A1
(43) Pub. Date: Oct. 30, 2025

(54) SOLID STATE FORMS

(71) Applicant: AMGEN INC., THOUSAND OAKS, CA (US)

(72) Inventors: DARREN L. REID, BELMONT, MA (US); VILMALI LOPEZ-MEJIAS, CAMBRIDGE, MA (US); LASZLO VISONTAI, WOODLAND HILLS, CA (US); JAKE S. NEWMAN, CANOGA PARK, CA (US); TAYLOR C. HARDIN, SHERMAN OAKS, CA (US); ABHIJEET S. MAURYA, THOUSAND OAKS, CA (US); ADAM N. SMITH, CAMBRIDGE, MA (US); PADMINI KAVURU ANANTHOJI, CAMBRIDGE, MA (US); JONATHON ANDREW REIFMAN, THOUSAND OAKS, CA (US)

(21) Appl. No.: 19/190,123

(22) Filed: Apr. 25, 2025

Related U.S. Application Data
(60) Provisional application No. 63/639,571, filed on Apr. 26, 2024.

(51) Int. Cl. C07D 211/60; A61K 31/44

(52) U.S. Cl. CPC C07D 211/60; A61K 31/44

(57) ABSTRACT

The present disclosure provides crystalline and amorphous HCl and HBr salt forms of (2R,3S)-2-(4-(cyclopentylamino)phenyl)-1-(2-fluoro-6-methylbenzoyl)-N-(4-methyl-3-(trifluoromethyl)phenyl)piperidine-3-carboxamide, pharmaceutical compositions, and methods of treating a disease mediated by C5aR inhibition.

### CROSS-REFERENCES TO RELATED APPLICATIONS

This application claims the benefit of priority under 35 U.S.C § 119(e) to U.S. Provisional Application Ser. No. 63/639,571 filed Apr. 26, 2024.

### FIELD

The present disclosure provides salt forms of (2R,3S)-2-(4-(cyclopentylamino)phenyl)-1-(2-fluoro-6-methylbenzoyl)-N-(4-methyl-3-(trifluoromethyl)phenyl)piperidine-3-carboxamide, (referred to herein as “Compound A”), including crystalline forms of anhydrous forms of salt forms of Compound A, solvate forms of salt forms of Compound A, amorphous forms of salt forms of Compound A, pharmaceutical compositions, and a method of treating a disease mediated by the C5a receptor (C5aR).

### BACKGROUND

The complement system plays an important role in the immune response, and the complement fragment C5a is an important component of the complement system that exerts diverse physiological functions through activation of the C5aR and associated downstream G protein and β-arrestin signaling pathways. Dysfunction of C5a and C5aR is associated with numerous inflammatory and immune-mediated diseases.

Compound A is a selective inhibitor of C5aR useful for the treatment of inflammatory diseases, including treatment of anti-neutrophil cytoplasmic autoantibody-associated vasculitis (also referred to as ANCA-associated vasculitis or AAV), including granulomatosis with polyangiitis (GPA) and microscopic polyangiitis (MPA), complement 3 glomerulopathy (C3G), hidradenitis suppurativa HS), or lupus nephritis, or any combination of the foregoing. United States Patent Application Publication Number 2016/0229802 A1, published on Aug. 17, 2016, discloses Compound A.

M any compounds can exist in different crystal forms, or polymorphs, which exhibit different physical, chemical, and spectroscopic properties.

Polymorphic forms of a compound are known in the pharmaceutical arts to affect, for example, the solubility, stability, flowability, plasticity, and compressibility of the compound, as well as the safety and efficacy of drug products comprising it. Therefore, the discovery of new polymorphs of a drug can provide a variety of advantages.

### SUMMARY

The present disclosure provides new salt forms of (2R,3S)-2-(4-(cyclopentylamino)phenyl)-1-(2-fluoro-6-methylbenzoyl)-N-(4-methyl-3-(trifluoromethyl)phenyl)piperidine-3-carboxamide (Compound A), including amorphous forms and crystalline forms, wherein the crystalline forms include anhydrous forms and solvate forms, pharmaceutical compositions of the new salt forms of Compound A, methods of making new salt forms of Compound A, and methods of treating a disease mediated by C5aR. The new salt forms of Compound A include HCl and HBr salt forms of Compound A. The new salt forms of Compound A can further the development of formulations for the treatment of disease mediated by C5aR, and may yield numerous formulation, manufacturing, and therapeutic benefits. Compound A has the following structure:

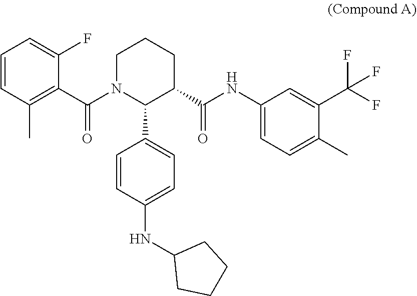 [SMILES: Cc1ccc(NC(=O)[C@H]2CCCN(C(=O)c3c(C)cccc3F)[C@H]2c2ccc(NC3CCCC3)cc2)cc1C(F)(F)F]

### BRIEF DESCRIPTION OF THE DRAWINGS

FIG. 1 shows XRPD data for an amorphous HCl salt Form 2 of Compound A. The powder X-ray diffraction pattern is characteristic of amorphous material with a broad amorphous halo and no distinct compound related diffraction peaks from 5-40° 2-theta.

FIGS. 2A and 2B shows DSC data for an amorphous HCl salt Form 2 of Compound A.

FIG. 3 shows TGA data for an amorphous HCl salt Form 2 of Compound A.

FIG. 4 shows XRPD data for an amorphous HBr salt Form 2 of Compound A. The powder X-ray diffraction pattern is characteristic of amorphous material with a broad amorphous halo and no distinct compound related diffraction peaks from 5-40° 2-theta.

FIGS. 5A and 5B shows DSC data for an amorphous HBr salt Form 2 of Compound A.

FIG. 6 shows TGA data for an amorphous HBr salt Form 2 of Compound A.

FIG. 7 shows XRPD data for the crystalline HCl salt Form 1 of Compound A.

FIG. 8 shows DSC data for crystalline HCl salt Form 1 of Compound A.

FIG. 9 shows TGA data for crystalline HCl salt Form 1 of Compound A.

FIG. 10 shows ¹³C ssNMR data for crystalline HCl salt Form 1 of Compound A.

FIG. 11 shows XRPD data for the crystalline HBr salt Form 1 of Compound A.

FIG. 12 shows DSC data for crystalline HBr salt Form 1 of Compound A.

FIG. 13 shows TGA data for crystalline HBr salt Form 1 of Compound A.

FIG. 14 shows ¹³C ssNMR data for crystalline HBr salt Form 1 of Compound A.

FIG. 15 shows XRPD data for the HCl salt toluene solvate Form 1 of Compound A.

FIG. 16 shows dissolution data for crystalline HBr salt Form 1 of Compound A, crystalline HCl salt orm 1 of Compound A, crystalline free base form of Compound A, and amorphous free base form of Compound A.

FIG. 17 shows TGA data for the HCl salt toluene solvate Form 1 of Compound A.

FIG. 18 shows XRPD data for the HCl salt toluene solvate Form 2 of Compound A.

FIG. 19 shows DSC data for the c HCl salt toluene solvate Form 2 of Compound A.

FIG. 20 shows TGA data for the HCl salt toluene solvate Form 2 of Compound A.

FIG. 21 shows XRPD data for the HBr salt toluene solvate Form 1 of Compound A.

FIG. 22 shows DSC data for the HBr salt toluene solvate Form 1 of Compound A.

FIG. 23 shows TGA data for the HBr salt toluene solvate Form 1 of Compound A.

FIG. 24 shows the overlay of the DSC and TGA plots of crystalline HCl salt Form 1 of Compound A.

FIG. 25 shows the overlay of the DSC and TGA plots of crystalline HBr salt Form 1 of Compound A.

FIG. 26 shows the DVS isotherm plot of crystalline HCl salt Form 1 of Compound A.

FIG. 27 shows the DVS isotherm plot of crystalline HBr salt Form 1 of Compound A.

FIG. 28 shows XRPD data for the crystalline HCl salt Form 3 of Compound A.

FIG. 29 shows DSC data for crystalline HCl salt Form 3 of Compound A.

FIG. 30 shows TGA data for crystalline HCl salt Form 3 of Compound A.

FIG. 31 shows ¹³C ssNMR data for crystalline HCl salt Form 3 of Compound A.

FIG. 32 shows XRPD data for the HCl salt TH F/H₂0 solvate Form 1 of Compound A.

FIG. 33 shows DSC data for the c HCl salt THF/H₂0 solvate Form 1 of Compound A.

FIG. 34 shows TGA data for the HCl salt THF/H₂0 Form 1 of Compound A.

FIGS. 35(a) and 35(b) show dissolution profiles of crystalline HCl Form 1 of Compound A of this disclosure compared to Napsylate, Tosylate, and Besylate salt forms of Compound A. The concentrations of the salts used are roughly double in FIG. 35(b) than in FIG. 35(a).

### DETAILED DESCRIPTION

### Abbreviations

The following abbreviations may be used herein:
CAN acetonitrile
DMSO dimethyl sulfoxide
HPLC high pressure liquid chromatography
Mg milligrams
Min minutes
mL milliliters
NMR nuclear magnetic resonance
ppm parts per million
ssNMR solid state nuclear magnetic resonance
TFA trifluoroacetic acid
UV ultraviolet

### Definitions

The term “Compound A” means (2R,3S)-2-(4-(cyclopentylamino)phenyl)-1-(2-fluoro-6-methylbenzoyl)-N-(4-methyl-3-(trifluoromethyl)phenyl)piperidine-3-carboxamide.

Recitation of ranges of values herein merely are intended to serve as a shorthand method of referring individually to each separate value falling within the range, unless otherwise indicated herein, and each separate value is incorporated into the specification as if it were individually recited herein. The use of any and all examples, or exemplary language (e.g., “such as”) provided herein, is intended to better illustrate the invention and is not a limitation on the scope of the invention unless otherwise claimed. No language in the specification should be construed as indicating any non-claimed element as essential to the practice of the invention.

The term “excipient” means any pharmaceutically acceptable additive, carrier, diluent, adjuvant, or other ingredient, other than the active pharmaceutical ingredient (API), which is typically included for formulation and/or administration to a patient.

The term “pharmaceutically acceptable” refers to a species or component that is generally safe, non-toxic, and neither biologically nor otherwise undesirable for use in a subject.

The term “pharmaceutically acceptable excipient” refers to a broad range of ingredients that may be combined with a compound, solvate, or salt (including all forms of said compound, solvates or salts) disclosed herein to prepare a pharmaceutically acceptable composition or formulation. Excipients include, for example, vehicles (e.g., solvents, dispersion media), coatings, isotonic and absorption delaying agents, diluents, colorants, glidants, disintegrants, flavoring agents, coatings, binders, sweeteners, lubricants, sorbents, and preservatives (e.g., antibacterial and antifungal agents)

The term “a disease mediated by C5aR” means inflammatory disorders and autoimmune disorders associated with the complement system and particular involving C5a and its receptor C5aR. C5aR is expressed on a broad spectrum of immune and non-immune cells and are involved in cellular functions and physiological processes during homeostasis and inflammation. Dysregulated C5a-mediated inflammation contributes to diseases such as anti-neutrophil cytoplasmic autoantibody-associated vasculitis (also referred to as ANCA-associated vasculitis or AAV), including granulomatosis with polyangiitis (GPA) and microscopic polyangiitis (MPA), complement 3 glomerulopathy (C3G), hidradenitis suppurativa (HS), and lupus nephritis, and others. Compound A, also known as AMG 569, received FDA approval as an adjunctive treatment of adult patients with severe active anti-neutrophil cytoplasmic autoantibody (ANCA)-associated vasculitis (granulomatosis with polyangiitis [GPA] and microscopic polyangiitis [MPA]) in combination with standard therapy including glucocorticoids. Treatment of C3G patients with Compound A in a Phase 2 Accolade clinical trial demonstrated statistically significant improvement in renal function as measured by eGFR compared to placebo over 26 weeks of blinded treatment. Treatment of HS patients with Compound in a Phase 3 Aurora clinical trial demonstrated statistically significant dose-dependent improvement in HiSCR (Hidradenitis Suppurativa Clinical Response) vs. placebo in pre-specified Hurley Stage III (severe HS) patients at 12 weeks.

As used herein, a condition is considered “responsive to C5a receptor modulation” if modulation of C5a receptor activity results in the reduction of inappropriate activity of a C5a receptor.

The term “patient” or “subject” refers to humans and other mammals. The term “mammal” as used herein includes, for example, humans, non-human primates, cattle, sheep, goats, pigs, horses, cats, dog, rabbits, rodents (e.g., rats or mice), and monkeys. Human subjects include neonates, infants, juveniles, adults, and geriatric subjects.

The term “therapeutically effective amount” as used herein refers to that amount of a compound disclosed herein that elicits a desired biological or medical response in a cell, a tissue, a system, or a subject.

The term “salt form(s) of Compound A” is meant to include crystalline forms of anhydrous forms of salt forms of Compound A, solvate forms of salt forms of Compound A, and amorphous forms of salt forms of Compound A. Salt forms of Compound A of this disclosure are also meant to include HBr and HCl salt forms of Compound A.

The term “amorphous halo” is an approximately bell-shaped maximum in the powder X-ray diffraction pattern of an amorphous substance.

As used herein and unless otherwise indicated, the terms “polymorph” and “polymorphic form” refer to solid crystalline forms of a compound or complex. Different polymorphs of the same compound can exhibit different physical, chemical and/or spectroscopic properties. Different physical properties include, but are not limited to stability (e.g., to heat or light), compressibility and density (important in formulation and product manufacturing), and dissolution rates (which can affect bioavailability). Differences in stability can result from changes in chemical reactivity (e.g., differential oxidation, such that a dosage form discolors more rapidly when comprised of one polymorph than when comprised of another polymorph) or mechanical characteristics (e.g., tablets crumble on storage as a kinetically favored polymorph converts to thermodynamically more stable polymorph) or both (e.g., tablets of one polymorph are more susceptible to breakdown at high humidity). Different physical properties of polymorphs can affect their processing. For example, one polymorph might be more likely to form solvates or might be more difficult to filter or wash free of impurities than another due to, for example, the shape or size distribution of particles of it.

As used herein and unless otherwise indicated, the term “substantially pure” when used to describe a polymorph of a compound means a solid form of the compound that comprises that polymorph and is substantially free of other polymorphs of the compound. A representative substantially pure polymorph comprises greater than about 80% by weight of one polymorphic form of the compound and less than about 20% by weight of other polymorphic forms of the compound. In another embodiment, a substantially pure polymorph comprises greater than about 90% by weight of one polymorphic form of the compound and less than about 10% by weight of the other polymorphic forms of the compound. In another embodiment, a substantially pure polymorph comprises greater than about 95% by weight of one polymorphic form of the compound and less than about 5% by weight of the other polymorphic forms of the compound. In yet another embodiment, a substantially pure polymorph comprises greater than about 97% by weight of one polymorphic forms of the compound and less than about 3% by weight of the other polymorphic forms of the compound.

### Embodiments

In some embodiments, the methods for treatment are directed to treating vasculitis, the methods comprise administering an effective amount of any of salt forms of Compound A described in this disclosure (or a pharmaceutical composition comprising the same) to a subject in need thereof. In certain embodiments the vasculitis is ANCA-associated vasculitis. In some embodiments, the methods comprising administering an effective amount of any of the salt forms of Compound A of this disclosure as an adjunctive treatment of adult patients with severe active anti-neutrophil cytoplasmic autoantibody (ANCA)-associated vasculitis (granulomatosis with polyangiitis [GPA] and microscopic polyangiitis [MPA]) in combination with standard therapy including glucocorticoids.

The salt forms of Compound A of this disclosure can be administered to a patient in a therapeutically effective amount. The salt forms of Compound A can be administered alone or as part of a pharmaceutically acceptable composition or formulation. In addition, the compounds or compositions can be administered all at once, as for example, by a bolus injection, multiple times, such as by a series of capsules, or delivered substantially uniformly over a period of time, as for example, using transdermal delivery. It is also noted that the dose of the compound can be varied over time. In another embodiment any of the salt forms of Compound A can be administered to a patient in an amount of about EQ 30 mg freebase twice daily. In another embodiment, any of the salt forms of Compound A is administered to a patient with food. In another embodiment, any of the salt forms of Compound A is administered to a patient without food.

In addition, any of the salt forms of Compound A of this disclosure can be administered alone, in combination with other pharmaceutically active compounds. The other pharmaceutically active compounds can be intended to treat the same disease or condition as the compounds of the present disclosure or a different disease or condition. If the patient is to receive or is receiving multiple pharmaceutically active compounds, the compounds can be administered simultaneously or sequentially. For example, in the case of capsules, the active compounds may be found in one capsule or in separate capsules, which can be administered at once or sequentially in any order. In addition, it should be recognized that the compositions may be different forms. For example, one or more compound may be delivered via a capsule, while another is administered via injection or orally as a syrup. All combinations, delivery methods and administration sequences are contemplated.

It is also noted that the salt forms of Compound A of this disclosure can be administered together. For example, substantially pure crystalline form of an HBr or HCl salt form of Compound A can be administered to a patient. Alternatively, about 90% by weight of crystalline form of an HBr or HCl salt form of Compound A can be administered with the remaining HBr or HCl salt form of Compound A present in other forms, such as the amorphous form. In another embodiment, 80% by weight of crystalline form of an HBr or HCl salt form of Compound A can be administered with the remaining HBr or HCl salt form of Compound A present in other forms, such as the amorphous form. All combinations are contemplated. In one embodiment of the disclosure, an HBr or HCl salt form of Compound A is administered to a patient in one substantially pure form. Those skilled in the art will appreciate the possible variations.

The salt forms of Compound A of this disclosure may be used in the manufacture of a medicament for the treatment of a disease mediated by C5aR, such as inflammatory and autoimmune diseases, including, but not limited to, anti-neutrophil cytoplasmic autoantibody-associated vasculitis (also referred to as ANCA-associated vasculitis or AAV), including granulomatosis with polyangiitis (GPA) and microscopic polyangiitis (MPA), complement 3 glomerulopathy (C3G), hidradenitis suppurativa (HS), or lupus nephritis, or any combination of the foregoing.

In still a further embodiment the disclosure relates to the use of a salt form of Compound A for the preparation of a medicament useful for the treatment of a disease mediated by C5aR, such as inflammatory and autoimmune diseases, including, but not limited to, as anti-neutrophil cytoplasmic autoantibody-associated vasculitis (also referred to as ANCA-associated vasculitis or AAV), including granulomatosis with polyangiitis (GPA) and microscopic polyangiitis (MPA), complement 3 glomerulopathy (C3G), hidradenitis suppurativa (HS), or lupus nephritis, or any combination of the foregoing.

Since one aspect of the present disclosure contemplates the treatment of a disease/condition with a combination of pharmaceutically active compounds that may be administered separately, the disclosure further relates to combining separate pharmaceutical compositions in kit form. The kit comprises two separate pharmaceutical compositions: a compound of the present disclosure, and a second pharmaceutical compound. The kit comprises a container for containing the separate compositions such as a divided bottle or a divided foil packet. Additional examples of containers include syringes, boxes and bags. Typically, the kit comprises directions for the use of the separate components. The kit form is particularly advantageous when the separate components are preferably administered in different dosage forms (e.g., oral and parenteral), are administered at different dosage intervals, or when titration of the individual components of the combination is desired by the prescribing physician or veterinarian.

### Formulation and Route of Administration

While it may be possible to administer a compound disclosed herein alone in the uses described, the compound administered normally will be present as an active ingredient in a pharmaceutical composition. Thus, further provided herein is a pharmaceutical composition comprising a the salt forms of Compound A of this disclosure, in combination with one or more pharmaceutically acceptable excipients and, if desired, other active ingredients. See, e.g., Remington: The Science and Practice of Pharmacy, Volume I and Volume 1l, twenty-second edition, edited by Loyd V. Allen Jr., Philadelphia, PA, Pharmaceutical Press, 2012; Pharmaceutical Dosage Forms (Vol. 1-3), Liberman et al., Eds., Marcel Dekker, New York, NY, 1992; Handbook of Pharmaceutical Excipients (3rd Ed.), edited by Arthur H. Kibbe, American Pharmaceutical Association, Washington, 2000; Pharmaceutical Formulation: The Science and Technology of Dosage Forms (Drug Discovery), first edition, edited by GD Tovey, Royal Society of Chemistry, 2018. In some cases, the pharmaceutical composition described herein comprises a therapeutically effective amount of a compound disclosed herein, or a pharmaceutically acceptable salt thereof.

The compound(s) disclosed herein may be administered by any suitable route in the form of a pharmaceutical composition adapted to such a route and in a dose effective for the treatment intended. The compounds and compositions presented herein may, for example, be administered orally, mucosally, topically, transdermally, rectally, pulmonarily, parentally, intranasally, intravascularly, intravenously, intraarterial, intraperitoneally, intrathecally, subcutaneously, sublingually, intramuscularly, intrasternally, vaginally or by infusion techniques, in dosage unit formulations containing conventional pharmaceutically acceptable excipients.

The pharmaceutical composition may be in the form of, for example, a tablet, chewable tablet, minitablet, caplet, pill, bead, hard capsule, soft capsule, gelatin capsule, granule, powder, lozenge, patch, cream, gel, sachet, microneedle array, syrup, flavored syrup, juice, drop, injectable solution, emulsion, microemulsion, ointment, aerosol, aqueous suspension, or oily suspension. In some cases, the pharmaceutical composition is made in the form of a dosage unit containing a particular amount of the active ingredient.

Thus, a further aspect of the disclosure is a pharmaceutical composition comprising one or more of the compounds disclosed herein, or a pharmaceutically acceptable salt thereof, and a pharmaceutically acceptable excipient. Further provided herein is a compound of the disclosure, or a pharmaceutically acceptable salt thereof, or the pharmaceutical composition described herein, for use as a medicament.

The salt forms of Compound A of this disclosure can be administered to a patient at dosage levels in the range of about EQ 10 mg free base to about EQ 200 mg free base per day. The specific dosage and dosage range that can be used depends on a number of factors, including the requirements of the patient, the severity of the condition or disease being treated, and the pharmacological activity of the compound being administered. The determination of dosage ranges and optimal dosages for a particular patient is within the ordinary skill in the art. In another embodiment the total daily dose administered to a patient is EQ 60 mg of Compound A freebase.

“EQ” designation in this disclosure is used in connection with salt drug products (e.g., the salt forms of Compound A) to indicate that the strength of such drug product is being expressed in terms of the equivalent strength of the active moiety (e.g., “EQ 60 mg freebase”).

Those skilled in the art will understand that the salt forms of Compound A of this disclosure may exist in one or more ionization states, which typically exists as zwitterions. While the name or structure for only a particular ionization state may be used, it is intended that all ionization states are encompassed by the present disclosure, unless stated otherwise.

The present disclosure is also intended to include salt forms of Compound A that are isotopically-labelled forms of Compound A wherein one or more atoms of Compound A are replaced by an atom having an atomic mass or mass number different from the atomic mass or mass number usually found in nature. Examples of isotopes that can be incorporated into Compound A of the disclosure include isotopes of hydrogen, carbon, nitrogen, oxygen, phosphorous, fluorine and chlorine, such as ²H, ³H, ¹³C, ¹⁴C, ¹⁵N, ¹⁶O, ¹⁷O, ³¹P, ³²p, ³⁵S, ¹⁸F, and ³⁶Cl.

Salt forms of Compound A of the present disclosure that contain the aforementioned isotopes and/or other isotopes of other atoms are within the scope of this disclosure. Certain isotopically-labelled compounds of the present disclosure, for example those into which radioactive isotopes such as ³H and ¹⁴C are incorporated, are useful in drug and/or substrate tissue distribution assays. By way of example, tritiated, i.e., ³H, and carbon-14, i.e., ¹⁴C, isotopes can be used for isotopic labelling because of their ease of preparation and detection. Further, substitution with heavier isotopes such as deuterium, i.e., ²H, can afford certain therapeutic advantages resulting from greater metabolic stability, for example increased in vivo half-life or reduced dosage requirements. Isotopically labelled compounds of this disclosure can generally be prepared by substituting a readily available isotopically labelled reagent for a non-isotopically labelled reagent.

Additional embodiments of this disclosure are described below:

### Additional Embodiments

Embodiment 1 of this disclosure relates to a crystalline salt form of (2R,3S)-2-(4-(cyclopentylamino)phenyl)-1-(2-fluoro-6-methylbenzoyl)-N-(4-methyl-3-(trifluoromethyl)phenyl)piperidine-3-carboxamide (Compound A), wherein the crystalline salt form of Compound A is a crystalline HCl salt form or a crystalline HBr salt form.

Embodiment 2 of this disclosure relates to the crystalline HCl salt form of Embodiment 1, wherein the crystalline HCl salt form is anhydrous.

Embodiment 3 of this disclosure relates to the crystalline HCl salt form of Embodiment 1 or Embodiment 2, wherein the crystalline HCl salt form is characterized by a powder X-ray diffraction pattern comprising at least one peak selected from 5.9, 7.3, 13.4, 13.7, 15.4, 16.3, 17.7, 18.7, 21.0, and 22.4±0.2 degrees 2 theta as measured by X-ray powder diffraction using an X-ray wavelength of 1.54 Å.

It is to be understood that the standard error in the embodiments of this disclosure described by the phrase “±0.2 degrees 2 theta as measured by X-ray powder diffraction using an X-ray wavelength of 1.54 Å” is meant to apply to each peak listing of the embodiment that precedes this phrase. Thus, for example, Embodiment 3 of this disclosure can also be phrased as the following which is meant to have exactly the same meaning: Embodiment 3 of this disclosure relates to the crystalline HCl salt form of Embodiment 1 or Embodiment 2, wherein the crystalline HCl salt form is characterized by a powder X-ray diffraction pattern comprising at least one peak selected from 5.9±0.2 degrees 2 theta, 7.3±0.2 degrees 2 theta, 13.4±0.2 degrees 2 theta, 13.7±0.2 degrees 2 theta, 15.4±0.2 degrees 2 theta, 16.3±0.2 degrees 2 theta, 17.7±0.2 degrees 2 theta, 18.7±0.2 degrees 2 theta, 21.0±0.2 degrees 2 theta, and 22 4±0.2 degrees 2 theta as measured by X-ray powder diffraction using an X-ray wavelength of 1.54 Å.

Embodiment 3(a) of this disclosure relates to the crystalline HCl salt form of Embodiment 3, wherein the crystalline HCl salt form is characterized by a powder X-ray diffraction pattern comprising peaks at 13.7, 15.4 and 16.3±0.2 degrees 2 theta as measured by X-ray powder diffraction using an X-ray wavelength of 1.54 Å.

Embodiment 3(b) of this disclosure relates to the crystalline HCl salt form of Embodiment 3, wherein the crystalline HCl salt form is characterized by a powder X-ray diffraction pattern comprising at least one peak selected from 5.9, 7.3, 13.4, 13.7, 15.4, 16.3, 17.7, 18.7, 21.0, and 22.4±0.1 degrees 2 theta as measured by X-ray powder diffraction using an X-ray wavelength of 1.54 Å.

Embodiment 3(c) of this disclosure relates to the crystalline HCl salt form of Embodiment 3(a), wherein the crystalline HCl salt form is characterized by a powder X-ray diffraction pattern comprising peaks at 13.7, 15.4 and 16.3±0.1 degrees 2 theta as measured by X-ray powder diffraction using an X-ray wavelength of 1.54 Å.

Embodiment 4 of this disclosure relates to the crystalline HCl salt form of Embodiment 1 or Embodiment 2, wherein the crystalline HCl salt form is characterized by a powder X-ray diffraction pattern comprising at least two peaks selected from 5.9, 7.3, 13.4, 13.7, 15.4, 16.3, 17.7, 18.7, 21.0, and 22.4±0.2 degrees 2 theta as measured by X-ray powder diffraction using an X-ray wavelength of 1.54 Å.

Embodiment 4(a) of this disclosure relates to the crystalline HCl salt form of Embodiment 4, wherein the crystalline HCl salt form is characterized by a powder X-ray diffraction pattern comprising at least two peaks selected from 5.9, 7.3, 13.4, 13.7, 15.4, 16.3, 17.7, 18.7, 21.0, and 22.4±0.1 degrees 2 theta as measured by X-ray powder diffraction using an X-ray wavelength of 1.54 Å.

Embodiment 5 of this disclosure relates to the crystalline HCl salt form of Embodiment 1 or Embodiment 2, wherein the crystalline HCl salt form is characterized by a powder X-ray diffraction pattern comprising at least three peaks selected from 5.9, 7.3, 13.4, 13.7, 15.4, 16.3, 17.7, 18.7, 21.0, and 22.4±0.2 degrees 2 theta as measured by X-ray powder diffraction using an X-ray wavelength of 1.54 Å.

Embodiment 5(a) of this disclosure relates to the crystalline HCl salt form of Embodiment 5, wherein the crystalline HCl salt form is characterized by a powder X-ray diffraction pattern comprising at least three peaks selected from 5.9, 7.3, 13.4, 13.7, 15.4, 16.3, 17.7, 18.7, 21.0, and 22.4±0.1 degrees 2 theta as measured by X-ray powder diffraction using an X-ray wavelength of 1.54 Å.

Embodiment 6 of this disclosure relates to the crystalline HCl salt form of Embodiment 1 or Embodiment 2, wherein the crystalline HCl salt form is characterized by a powder X-ray diffraction pattern comprising peaks at 5.9, 7.3, 13.4, 13.7, 15.4, 16.3, 17.7, 18.7, 21.0, and 22.4±0.2 degrees 2 theta as measured by X-ray powder diffraction using an X-ray wavelength of 1.54 Å.

Embodiment 6(a) of this disclosure relates to the crystalline HCl salt form of Embodiment 6, wherein the crystalline HCl salt form is characterized by a powder X-ray diffraction pattern comprising peaks at 5.9, 7.3, 13.4, 13.7, 15.4, 16.3, 17.7, 18.7, 21.0, and 22.4±0.1 degrees 2 theta as measured by X-ray powder diffraction using an X-ray wavelength of 1.54 Å.

Embodiment 7 of this disclosure relates to the crystalline HCl salt form of Embodiment 1 or Embodiment 2, wherein the crystalline HCl salt form is characterized by a powder X-ray diffraction pattern comprising at least three peaks selected from 5.9, 6.7, 7.3, 10.3, 11.5, 12.9, 13.4, 13.7, 15.4, 16.3, 17.7, 18.7, 19.8, 21.0, 21.9, 22.4, 23.1, 23.8, 25.7, and 28.3±0.2 degrees 2 theta as measured by X-ray powder diffraction using an X-ray wavelength of 1.54 Å.

Embodiment 7(a) of this disclosure relates to the crystalline HCl salt form of Embodiment 7, wherein the crystalline HCl salt form is characterized by a powder X-ray diffraction pattern comprising at least three peaks selected from 5.9, 6.7, 7.3, 10.3, 11.5, 12.9, 13.4, 13.7, 15.4, 16.3, 17.7, 18.7, 19.8, 21.0, 21.9, 22.4, 23.1, 23.8, 25.7, and 28.3±0.1 degrees 2 theta as measured by X-ray powder diffraction using an X-ray wavelength of 1.54 Å.

Embodiment 8 of this disclosure relates to the crystalline HCl salt form of Embodiment 1 or Embodiment 2, wherein the crystalline HCl salt form is characterized by a powder X-ray diffraction pattern comprising at least five peaks selected from 5.9, 6.7, 7.3, 10.3, 11.5, 12.9, 13.4, 13.7, 15.4, 16.3, 17.7, 18.7, 19.8, 21.0, 21.9, 22.4, 23.1, 23.8, 25.7, and 28.3±0.2 degrees 2 theta as measured by X-ray powder diffraction using an X-ray wavelength of 1.54 Å.

Embodiment 8(a) of this disclosure relates to the crystalline HCl salt form of Embodiment 8, wherein the crystalline HCl salt form is characterized by a powder X-ray diffraction pattern comprising at least five peaks selected from 5.9, 6.7, 7.3, 10.3, 11.5, 12.9, 13.4, 13.7, 15.4, 16.3, 17.7, 18.7, 19.8, 21.0, 21.9, 22.4, 23.1, 23.8, 25.7, and 28.3±0.1 degrees 2 theta as measured by X-ray powder diffraction using an X-ray wavelength of 1.54 Å.

Embodiment 9 of this disclosure relates to the crystalline HCl salt form of Embodiment 1 or Embodiment 2, wherein the crystalline HCl salt form is characterized by a powder X-ray diffraction pattern comprising at least seven peaks selected from 5.9, 6.7, 7.3, 10.3, 11.5, 12.9, 13.4, 13.7, 15.4, 16.3, 17.7, 18.7, 19.8, 21.0, 21.9, 22.4, 23.1, 23.8, 25.7, and 28.3±0.2 degrees 2 theta as measured by X-ray powder diffraction using an X-ray wavelength of 1.54 Å.

Embodiment 9(a) of this disclosure relates to the crystalline HCl salt form of Embodiment 9, wherein the crystalline HCl salt form is characterized by a powder X-ray diffraction pattern comprising at least seven peaks selected from 5.9, 6.7, 7.3, 10.3, 11.5, 12.9, 13.4, 13.7, 15.4, 16.3, 17.7, 18.7, 19.8, 21.0, 21.9, 22.4, 23.1, 23.8, 25.7, and 28.3±0.1 degrees 2 theta as measured by X-ray powder diffraction using an X-ray wavelength of 1.54 Å.

Embodiment 10 of this disclosure relates to the crystalline HCl salt form of Embodiment 1 or Embodiment 2, wherein the crystalline HCl salt form is characterized by a powder X-ray diffraction pattern comprising peaks at 5.9, 6.7, 7.3, 10.3, 11.5, 12.9, 13.4, 13.7, 15.4, 16.3, 17.7, 18.7, 19.8, 21.0, 21.9, 22.4, 23.1, 23.8, 25.7, and 28.3±0.2 degrees 2 theta as measured by X-ray powder diffraction using an X-ray wavelength of 1.54 Å.

Embodiment 10(a) of this disclosure relates to the crystalline HCl salt form of Embodiment 1 or Embodiment 2, wherein the crystalline HCl salt form is characterized by a powder X-ray diffraction pattern comprising peaks at 5.9, 6.7, 7.3, 10.3, 11.5, 12.9, 13.4, 13.7, 15.4, 16.3, 17.7, 18.7, 19.8, 21.0, 21.9, 22.4, 23.1, 23.8, 25.7, and 28.3±0.1 degrees 2 theta as measured by X-ray powder diffraction using an X-ray wavelength of 1.54 Å.

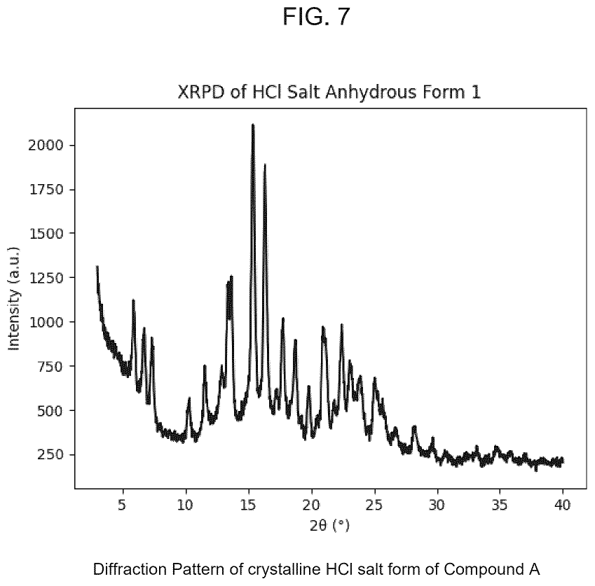

Embodiment 11 of this disclosure relates to the crystalline HCl salt form of Embodiment 1 or Embodiment 2, wherein the crystalline HCl salt form is characterized by the powder X-ray diffraction pattern substantially as shown in FIG. 7 as measured by X-ray powder diffraction using an X-ray wavelength of 1.54 Å.

Embodiment 12 of this disclosure relates to the crystalline HCl salt form of any one of Embodiments 1-11, wherein the crystalline HCl salt form of is characterized by a differential scanning calorimetry thermogram comprising an endotherm with an onset of about 192±1° C.

Embodiment 13 of this disclosure relates to the crystalline HCl salt form of any one of Embodiments 1-12, wherein the crystalline HCl salt is characterized by a thermogravimetric analysis thermogram comprising a weight loss of about 0.33%±0.1% when heated from about 25° C. to about 125° C.

Embodiment 14 of this disclosure relates to the crystalline HCl salt form of Embodiment 13, wherein the thermogravimetric analysis thermogram further comprising a weight loss of about 5.6%±0.5% from about 125° C. to about 200° C.

Embodiment 15 of this disclosure relates to the crystalline HCl salt of any one of Embodiments 1-14, or any sub-embodiments thereof, wherein the crystalline HCl salt form is characterized by ¹³C solid state NMR comprising at least three peaks selected from peaks at approximately 170, 166, 160, 158, 140, 137, 135, 132, 129, 126, 124, 116, 115, 111, 65, 60, 54, 52, 49, 46, 44, 30, 28, 25, and 19 ppm.

Embodiment 16 of this disclosure relates to the crystalline HCl salt of any one of Embodiments 1-14, or any sub-embodiments thereof, wherein the crystalline HCl salt form is characterized by ¹³C solid state NMR comprising at least five peaks selected from peaks at approximately 170, 166, 160, 158, 140, 137, 135, 132, 129, 126, 124, 116, 115, 111, 65, 60, 54, 52, 49, 46, 44, 30, 28, 25, and 19 ppm.

Embodiment 17 of this disclosure relates to the crystalline HCl salt of any one of Embodiments 1-14, or any sub-embodiments thereof, wherein the crystalline HCl salt form is characterized by ¹³C solid state NMR comprising at least seven peaks selected from peaks at approximately 170, 166, 160, 158, 140, 137, 135, 132, 129, 126, 124, 116, 115, 111, 65, 60, 54, 52, 49, 46, 44, 30, 28, 25, and 19 ppm.

Embodiment 18 of this disclosure relates to the crystalline HCl salt of any one of Embodiments 1-14, or any sub-embodiments thereof, wherein the crystalline HCl salt form is characterized by ¹³C solid state NMR comprising peaks at approximately 170, 166, 160, 158, 140, 137, 135, 132, 129, 126, 124, 116, 115, 111, 65, 60, 54, 52, 49, 46, 44, 30, 28, 25, and 19 ppm.

Embodiment 19 of this disclosure relates to the crystalline HCl salt form of any one of Embodiments 1-18, or any sub-embodiments thereof, wherein the crystalline HCl salt form is substantially free of other crystalline or amorphous forms.

Embodiment 20 of this disclosure relates to the crystalline HCl salt form of Embodiment 19, wherein the amount of other crystalline or amorphous forms is 5% (w/w) or less.

Embodiment 21 of this disclosure relates to the crystalline HBr salt form of Embodiment 1, wherein the crystalline HBr salt form is anhydrous.

Embodiment 22 of this disclosure relates to the crystalline HBr salt form of Embodiment 1 or Embodiment 21, wherein the crystalline HBr salt form is characterized by a powder X-ray diffraction pattern comprising at least one peak selected from 7.5, 12.8, 13.2, 13.6, 15.3, 17.9, 18.8, 20.9, 21.3, 22.9, and 25.2±0.2 degrees 2 theta as measured by X-ray powder diffraction using an X-ray wavelength of 1.54 Å.

It is to be understood that the standard error in the embodiments of this disclosure described by the phrase “±0.2 degrees 2 theta as measured by X-ray powder diffraction using an X-ray wavelength of 1.54 Å” is meant to apply to each peak listing of the embodiment that precedes this phrase. Thus, for example, Embodiment 22 of this disclosure can also be phrased as the following which is meant to have exactly the same meaning: Embodiment 22 of this disclosure relates to the crystalline HBr salt form of Embodiment 1 or Embodiment 21, wherein the crystalline HBr salt form is characterized by a powder X-ray diffraction pattern comprising at least one peak selected from 7.5±0.2 degrees 2 theta, 12.8±0.2 degrees 2 theta, 13.2±0.2 degrees 2 theta, 13.6±0.2 degrees 2 theta, 15.3±0.2 degrees 2 theta, 17.9±0.2 degrees 2 theta, 18.8±0.2 degrees 2 theta, 20.9±0.2 degrees 2 theta, 21.3±0.2 degrees 2 theta, 22.9±0.2 degrees 2 theta, and 25.2±0.2 degrees 2 theta as measured by X-ray powder diffraction using an X-ray wavelength of 1.54 Å.

Embodiment 22(a) of this disclosure relates to the crystalline HBr salt form of Embodiment 22, wherein the crystalline HBr salt form is characterized by a powder X-ray diffraction pattern comprising at least one peak selected from 7.5, 12.8, 13.2, 13.6, 15.3, 17.9, 18.8, 20.9, 21.3, 22.9, and 25.2±0.1 degrees 2 theta as measured by X-ray powder diffraction using an X-ray wavelength of 1.54 Å.

Embodiment 22(b) of this disclosure relates to the crystalline HBr salt form of Embodiment 22, wherein the crystalline HBr salt form is characterized by a powder X-ray diffraction pattern comprising peaks at 13.2, 15.3 and 20.9±0.2 degrees 2 theta as measured by X-ray powder diffraction using an X-ray wavelength of 1.54 Å.

Embodiment 22(c) of this disclosure relates to the crystalline HBr salt form of Embodiment 22, wherein the crystalline HBr salt form is characterized by a powder X-ray diffraction pattern comprising peaks at 13.2, 15.3 and 20.9±0.1 degrees 2 theta as measured by X-ray powder diffraction using an X-ray wavelength of 1.54 Å.

Embodiment 23 of this disclosure relates to the crystalline HBr salt form of Embodiment 1 or Embodiment 21, wherein the crystalline HBr salt form is characterized by a powder X-ray diffraction pattern comprising peaks at 7.5, 13.2, and 15.3±0.2 degrees 2 theta as measured by X-ray powder diffraction using an X-ray wavelength of 1.54 Å.

Embodiment 23(a) of this disclosure relates to the crystalline HBr salt form of Embodiment 23, wherein the crystalline HBr salt form is characterized by a powder X-ray diffraction pattern comprising peaks at 7.5, 13.2, and 15.3±0.1 degrees 2 theta as measured by X-ray powder diffraction using an X-ray wavelength of 1.54 Å.

Embodiment 24 of this disclosure relates to the crystalline HBr salt form of Embodiment 1 or Embodiment 21, wherein the crystalline HBr salt form is characterized by a powder X-ray diffraction pattern comprising at least three peaks selected from 7.5, 12.8, 13.2, 13.6, 15.3, 17.9, 18.8, 20.9, 21.3, 22.9, and 25.2±0.2 degrees 2 theta as measured by X-ray powder diffraction using an X-ray wavelength of 1.54 Å.

Embodiment 24(a) of this disclosure relates to the crystalline HBr salt form of Embodiment 24, wherein the crystalline HBr salt form is characterized by a powder X-ray diffraction pattern comprising at least three peaks selected from 7.5, 12.8, 13.2, 13.6, 15.3, 17.9, 18.8, 20.9, 21.3, 22.9, and 25.2±0.1 degrees 2 theta as measured by X-ray powder diffraction using an X-ray wavelength of 1.54 Å.

Embodiment 25 of this disclosure relates to the crystalline HBr salt form of Embodiment 1 or Embodiment 21, wherein the crystalline anhydrous HBr salt form is characterized by a powder X-ray diffraction pattern comprising peaks at 7.5, 12.8, 13.2, 13.6, 15.3, 17.9, 18.8, 20.9, 21.3, 22.9, and 25.2±0.2 degrees 2 theta as measured by X-ray powder diffraction using an X-ray wavelength of 1.54 Å.

Embodiment 25(a) of this disclosure relates to the crystalline HBr salt form of Embodiment 25, wherein the crystalline anhydrous HBr salt form is characterized by a powder X-ray diffraction pattern comprising peaks at 7.5, 12.8, 13.2, 13.6, 15.3, 17.9, 18.8, 20.9, 21.3, 22.9, and 25.2±0.1 degrees 2 theta as measured by X-ray powder diffraction using an X-ray wavelength of 1.54 Å.

Embodiment 26 of this disclosure relates to the crystalline HBr salt form of Embodiment 1 or Embodiment 21, wherein the crystalline HBr salt form is characterized by a powder X-ray diffraction pattern comprising at least three peaks selected from 7.5, 10.1, 11.8, 12.5, 12.8, 13.2, 13.6, 15.3, 16.0, 16.4, 17.4, 17.9, 18.8, 19.8, 20.3, 20.9, 21.3, 22.0, 22.9, 23.3, 23.8, 24.7, 25.2, 26.4, 27.8, 28.9, 30.0, 32.8, and 34.7±0.2 degrees 2 theta as measured by X-ray powder diffraction using an X-ray wavelength of 1.54 Å.

Embodiment 26(a) of this disclosure relates to the crystalline HBr salt form of Embodiment 26, wherein the crystalline HBr salt form is characterized by a powder X-ray diffraction pattern comprising at least three peaks selected from 7.5, 10.1, 11.8, 12.5, 12.8, 13.2, 13.6, 15.3, 16.0, 16.4, 17.4, 17.9, 18.8, 19.8, 20.3, 20.9, 21.3, 22.0, 22.9, 23.3, 23.8, 24.7, 25.2, 26.4, 27.8, 28.9, 30.0, 32.8, and 34.7±0.1 degrees 2 theta as measured by X-ray powder diffraction using an X-ray wavelength of 1.54 Å.

Embodiment 27 of this disclosure relates to the crystalline HBr salt form of Embodiment 1 or Embodiment 21, wherein the crystalline HBr salt form is characterized by a powder X-ray diffraction pattern comprising at least five peaks selected from 7.5, 10.1, 11.8, 12.5, 12.8, 13.2, 13.6, 15.3, 16.0, 16.4, 17.4, 17.9, 18.8, 19.8, 20.3, 20.9, 21.3, 22.0, 22.9, 23.3, 23.8, 24.7, 25.2, 26.4, 27.8, 28.9, 30.0, 32.8, and 34.7±0.2 degrees 2 theta as measured by X-ray powder diffraction using an X-ray wavelength of 1.54 Å.

Embodiment 27(a) of this disclosure relates to the crystalline HBr salt form of Embodiment 27, wherein the crystalline HBr salt form is characterized by a powder X-ray diffraction pattern comprising at least five peaks selected from 7.5, 10.1, 11.8, 12.5, 12.8, 13.2, 13.6, 15.3, 16.0, 16.4, 17.4, 17.9, 18.8, 19.8, 20.3, 20.9, 21.3, 22.0, 22.9, 23.3, 23.8, 24.7, 25.2, 26.4, 27.8, 28.9, 30.0, 32.8, and 34.7±0.1 degrees 2 theta as measured by X-ray powder diffraction using an X-ray wavelength of 1.54 Å.

Embodiment 28 of this disclosure relates to the crystalline HBr salt form of Embodiment 1 or Embodiment 21, wherein the crystalline HBr salt form is characterized by a powder X-ray diffraction pattern comprising at least seven peaks selected from 7.5, 10.1, 11.8, 12.5, 12.8, 13.2, 13.6, 15.3, 16.0, 16.4, 17.4, 17.9, 18.8, 19.8, 20.3, 20.9, 21.3, 22.0, 22.9, 23.3, 23.8, 24.7, 25.2, 26.4, 27.8, 28.9, 30.0, 32.8, and 34.7±0.2 degrees 2 theta as measured by X-ray powder diffraction using an X-ray wavelength of 1.54 Å.

Embodiment 28(a) of this disclosure relates to the crystalline HBr salt form of Embodiment 28, wherein the crystalline HBr salt form is characterized by a powder X-ray diffraction pattern comprising at least seven peaks selected from 7.5, 10.1, 11.8, 12.5, 12.8, 13.2, 13.6, 15.3, 16.0, 16.4, 17.4, 17.9, 18.8, 19.8, 20.3, 20.9, 21.3, 22.0, 22.9, 23.3, 23.8, 24.7, 25.2, 26.4, 27.8, 28.9, 30.0, 32.8, and 34.7±0.1 degrees 2 theta as measured by X-ray powder diffraction using an X-ray wavelength of 1.54 Å.

Embodiment 29 of this disclosure relates to the crystalline HBr salt form of Embodiment 1 or Embodiment 21, wherein the crystalline HBr salt form is characterized by a powder X-ray diffraction pattern comprising peaks at 7.5, 10.1, 11.8, 12.5, 12.8, 13.2, 13.6, 15.3, 16.0, 16.4, 17.4, 17.9, 18.8, 19.8, 20.3, 20.9, 21.3, 22.0, 22.9, 23.3, 23.8, 24.7, 25.2, 26.4, 27.8, 28.9, 30.0, 32.8, and 34.7±0.2 degrees 2 theta as measured by X-ray powder diffraction using an X-ray wavelength of 1.54 Å.

Embodiment 29(a) of this disclosure relates to the crystalline HBr salt form of Embodiment 20, wherein the crystalline HBr salt form is characterized by a powder X-ray diffraction pattern comprising peaks at 7.5, 10.1, 11.8, 12.5, 12.8, 13.2, 13.6, 15.3, 16.0, 16.4, 17.4, 17.9, 18.8, 19.8, 20.3, 20.9, 21.3, 22.0, 22.9, 23.3, 23.8, 24.7, 25.2, 26.4, 27.8, 28.9, 30.0, 32.8, and 34.7±0.1 degrees 2 theta as measured by X-ray powder diffraction using an X-ray wavelength of 1.54 Å.

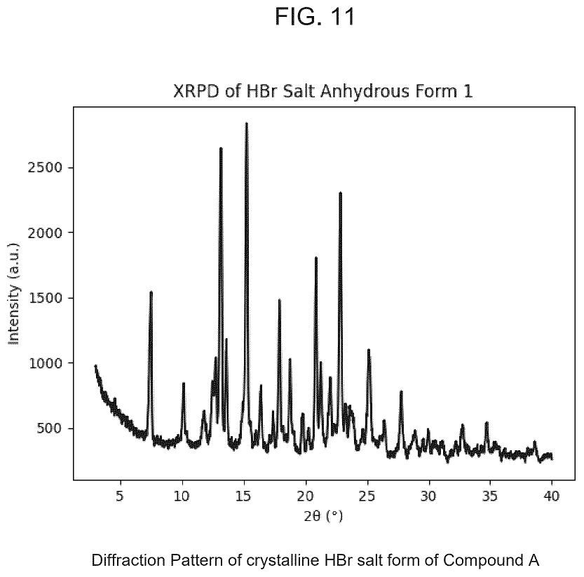

Embodiment 30 of this disclosure relates to the crystalline HBr salt form of Embodiment 1 or Embodiment 21, wherein the crystalline HBr salt form is characterized by the powder X-ray diffraction pattern substantially as shown in FIG. 11 as measured by X-ray powder diffraction using an X-ray wavelength of 1.54 Å.

Embodiment 31 of this disclosure relates to the crystalline HBr salt form of any one of Embodiments 1 or 21-30, or any sub-embodiments thereof, wherein the crystalline HBr salt form is characterized by a differential scanning calorimetry thermogram comprising an endotherm with an onset of about 201±1° C.

Embodiment 32 of this disclosure relates to the crystalline HBr salt form of any one of Embodiments 1 or 21-31, or any sub-embodiments thereof, wherein the crystalline HBr salt is characterized by a thermogravimetric analysis thermogram comprising a weight loss of about 0.35%±0.1% when heated from about 25° C. to about 150° C.

Embodiment 33 of this disclosure relates to the crystalline HBr salt of any one of Embodiments 1 or 21-32, or any sub-embodiments thereof, wherein the crystalline HBr salt form is characterized by ¹³C solid state NMR comprising at least three peaks selected from peaks at approximately 170, 166, 160, 159, 158, 157, 141, 137, 135, 132, 129, 126, 124, 117, 115, 111, 65, 60, 52, 46, 44, 30, 28, 24, and 19 ppm.

Embodiment 34 of this disclosure relates to the crystalline HBr salt of any one of Embodiments 1 or 21-32, or any sub-embodiments thereof, wherein the crystalline HBr salt form is characterized by ¹³C solid state NMR comprising at least five peaks selected from peaks at approximately 170, 166, 160, 159, 158, 157, 141, 137, 135, 132, 129, 126, 124, 117, 115, 111, 65, 60, 52, 46, 44, 30, 28, 24, and 19 ppm.

Embodiment 35 of this disclosure relates to the crystalline HBr salt of any one of Embodiments 1 or 21-32, or any sub-embodiments thereof, wherein the crystalline HBr salt form is characterized by ¹³C solid state NMR comprising at least seven peaks selected from peaks at approximately 170, 166, 160, 159, 158, 157, 141, 137, 135, 132, 129, 126, 124, 117, 115, 111, 65, 60, 52, 46, 44, 30, 28, 24, and 19 ppm.

Embodiment 36 of this disclosure relates to the crystalline HBr salt of any one of Embodiments 1 or 21-32, or any sub-embodiments thereof, wherein the crystalline HBr salt form is characterized by ¹³C solid state NMR comprising peaks at approximately 170, 166, 160, 159, 158, 157, 141, 137, 135, 132, 129, 126, 124, 117, 115, 111, 65, 60, 52, 46, 44, 30, 28, 24, and 19 ppm.

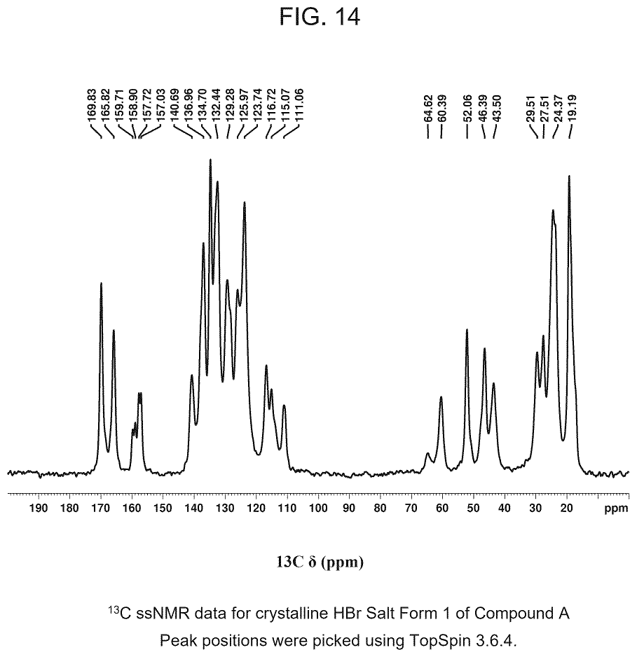

Embodiment 37 of this disclosure relates to the crystalline HBr salt form of any one of Embodiments 1 or 21-32, or any sub-embodiments thereof, wherein the crystalline HBr salt form is characterized by ¹³C solid state NMR substantially as depicted in FIG. 14.

Embodiment 38 of this disclosure relates to the crystalline HBr salt form of any one of Embodiments 1 or 21-37, or any sub-embodiments thereof, wherein the crystalline HBr salt form is substantially free of other crystalline or amorphous forms.

Embodiment 39 of this disclosure relates to the crystalline HBr salt form of Embodiment 38, wherein the amount of other crystalline or amorphous forms is 5% (w/w) or less.

Embodiment 40 of this disclosure relates to a pharmaceutical composition comprising the crystalline salt form of Compound A of any one of Embodiments 1-39 or any sub-embodiments thereof, and a pharmaceutically acceptable excipient.

Embodiment 41 of this disclosure relates to a method of treating a disease mediated by C5aR in a subject in need of treatment, the method comprising administering to the subject a therapeutically effective amount of the crystalline salt form of Compound A of any one of Embodiments 1-39, or any sub-embodiments thereof, or the pharmaceutical composition of Embodiment 40.

Embodiment 42 of this disclosure relates to the method of Embodiment 41, wherein the disease mediated by C5aR is anti-neutrophil cytoplasmic autoantibody-associated vasculitis, complement 3 glomerulopathy, hidradenitis suppurativa, or lupus nephritis, or any combination of the foregoing.

Embodiment 43 of this disclosure relates to a method of Embodiment 42, wherein the disease mediated by C5aR is anti-neutrophil cytoplasmic autoantibody-associated vasculitis.

Embodiment 44 of this disclosure relates to the method of any one of Embodiments 41-43, wherein the compound is administered at a total daily dose of EQ 60 mg free base of Compound A or EQ 30 mg freebase of compound A twice daily.

Embodiment 44(a) of this disclosure relates to any one of Embodiments 41-44, further comprising simultaneous, separate, or sequential administration of an effective amount of a second compound, wherein the second compound is one or more anti-inflammatory and/or immunosuppressive agents. Non-limiting examples of the second compound include prednisone, rituximab, cyclophosphamide, or any combination of the foregoing.

Embodiment 45 of this disclosure relates to the crystalline HCl salt form of Embodiment 1, wherein the crystalline HCl salt form is a crystalline toluene solvate.

Embodiment 46 of this disclosure relates to the crystalline toluene solvate of Claim 45, wherein the crystalline toluene solvate is characterized by a powder X-ray diffraction pattern comprising at least three peaks selected from 6.3, 13.5, 16.7, 17.1, 18.9, 20.5, 23.4, and 24.1±0.2 degrees 2 theta as measured by X-ray powder diffraction using an X-ray wavelength of 1.54 Å.

Embodiment 46(a) of this disclosure relates to the crystalline toluene solvate of Claim 45, wherein the crystalline toluene solvate is characterized by a powder X-ray diffraction pattern comprising at least three peaks selected from 6.3, 13.5, 16.7, 17.1, 18.9, 20.5, 23.4, and 24.1±0.1 degrees 2 theta as measured by X-ray powder diffraction using an X-ray wavelength of 1.54 Å.

Embodiment 47 of this disclosure relates to the crystalline toluene solvate of Embodiment 46, further characterized by one or more peaks selected from 5.1, 9.8, 11.9, 12.6, 14.4, 15.3, 17.8, 18.2, 19.5, 21.4, 22.6, and 24.9±0.2 degrees 2 theta as measured by X-ray powder diffraction using an X-ray wavelength of 1.54 Å.

Embodiment 47(a) of this disclosure relates to the crystalline toluene solvate of Embodiment 46, further characterized by one or more peaks selected from 5.1, 9.8, 11.9, 12.6, 14.4, 15.3, 17.8, 18.2, 19.5, 21.4, 22.6, and 24.9±0.1 degrees 2 theta as measured by X-ray powder diffraction using an X-ray wavelength of 1.54 Å.

Embodiment 48 of this disclosure relates to the crystalline toluene solvate of Embodiment 45, wherein the crystalline toluene solvate is characterized by a powder X-ray diffraction pattern comprising at least 3 peaks selected at 5.2, 11.9, 13.7, 14.9, 15.6, and 16.4±0.2 degrees 2 theta as measured by X-ray powder diffraction using an X-ray wavelength of 1.54 Å.

Embodiment 48(a) of this disclosure relates to the crystalline toluene solvate of Embodiment 45, wherein the crystalline toluene solvate is characterized by a powder X-ray diffraction pattern comprising at least 3 peaks selected at 5.2, 11.9, 13.7, 14.9, 15.6, and 16.4±0.1 degrees 2 theta as measured by X-ray powder diffraction using an X-ray wavelength of 1.54 Å.

Embodiment 49 of this disclosure relates to the crystalline toluene solvate of Embodiment 48, further characterized by one or more peaks selected from 6.6, 8.1, 8.6, 10.3, 17.2, 18.0, 18.8, 21.6, 22.1, 22.8, 23.4, and 24.3, ±0.2 degrees 2 theta as measured by X-ray powder diffraction using an X-ray wavelength of 1.54 Å.

Embodiment 49(a) of this disclosure relates to the crystalline toluene solvate of Embodiment 48, further characterized by one or more peaks selected from 6.6, 8.1, 8.6, 10.3, 17.2, 18.0, 18.8, 21.6, 22.1, 22.8, 23.4, and 24.3, ±0.1 degrees 2 theta as measured by X-ray powder diffraction using an X-ray wavelength of 1.54 Å.

Embodiment 50 of this disclosure relates to the crystalline HBr salt form of Embodiment 1, wherein the crystalline HBr salt form is a crystalline toluene solvate.

Embodiment 51 of this disclosure relates to the e crystalline toluene solvate of Embodiment 50, wherein the crystalline toluene solvate is characterized by a powder X-ray diffraction pattern comprising at least three peaks selected at 6.4, 8.4, 11.7, 12.9, 13.6, 15.3, 16.2, 21.8, and 24.0±0.2 degrees 2 theta as measured by X-ray powder diffraction using an X-ray wavelength of 1.54 Å.

Embodiment 51(a) of this disclosure relates to the e crystalline toluene solvate of Embodiment 50, wherein the crystalline toluene solvate is characterized by a powder X-ray diffraction pattern comprising at least three peaks selected at 6.4, 8.4, 11.7, 12.9, 13.6, 15.3, 16.2, 21.8, and 24.0±0.1 degrees 2 theta as measured by X-ray powder diffraction using an X-ray wavelength of 1.54 Å.

Embodiment 52 of this disclosure relates to the crystalline toluene solvate of Embodiment 51, further characterized by one or more of the following:
(a) one or more peaks selected 10.1, 16.9, 17.6, 18.4, 19.2, 19.8, 20.4, 21, 21.4, 22.6, 23.0, and 24.9±0.2 degrees 2 theta as measured by X-ray powder diffraction using an X-ray wavelength of 1.54 Å;
(b) a differential scanning calorimetry thermogram comprising an endotherm with an onset of about 195±1° C.; or
(c) a thermogravimetric analysis thermogram comprising a weight loss of about 8%±1% when heated from about 25° C. to about 150° C.

Embodiment 52(a) of this disclosure relates to the crystalline toluene solvate of Embodiment 51, further characterized by one or more of the following:
a) one or more peaks selected 10.1, 16.9, 17.6, 18.4, 19.2, 19.8, 20.4, 21, 21.4, 22.6, 23.0, and 24.9±0.1 degrees 2 theta as measured by X-ray powder diffraction using an X-ray wavelength of 1.54 Å;
b) a differential scanning calorimetry thermogram comprising an endotherm with an onset of about 195±1° C.; or
c) a thermogravimetric analysis thermogram comprising a weight loss of about 8%±1% when heated from about 25° C. to about 150° C.

Embodiment 53 of this disclosure relates to an amorphous salt form of (2R,3S)-2-(4-(cyclopentylamino)phenyl)-1-(2-fluoro-6-methylbenzoyl)-N-(4-methyl-3-(trifluoromethyl)phenyl)piperidine-3-carboxamide (Compound A), wherein the amorphous salt form of Compound A is an amorphous HCl salt form or an amorphous HBr salt form.

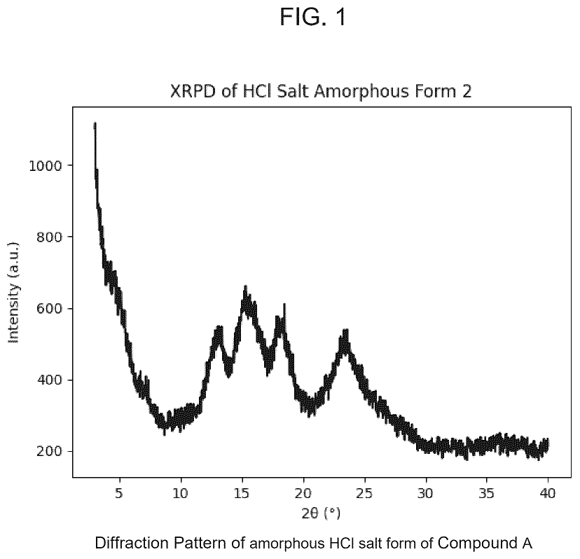

Embodiment 54 of this disclosure relates to the amorphous HCl salt form of Embodiment 53 characterized by one or more of the following:
(a) a powder X-ray diffraction pattern substantially as shown in FIG. 1 using an X-ray wavelength of 1.54 Å;
(b) a modulated differential scanning calorimetry thermogram comprising a glass transition temperature of about 74±1° C.; or
(c) a thermogravimetric analysis thermogram comprising a weight loss of about 0.6%±0.1% when heated from about 25° C. to about 125° C.

Embodiment 55 of this disclosure relates to the amorphous HCl salt form of Embodiment 54, wherein the amorphous HCl salt form is substantially free of other crystalline or amorphous forms.

Embodiment 56 of this disclosure relates to the amorphous HCl salt form of Embodiment 54, wherein the amount of other crystalline or amorphous forms is 5% (w/w) or less.

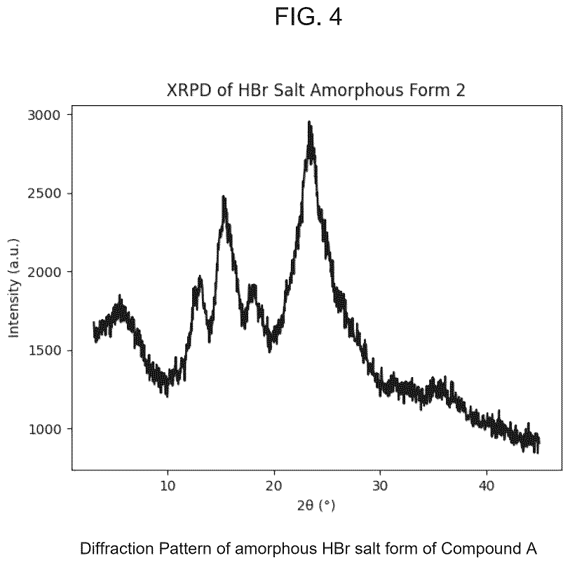

Embodiment 57 of this disclosure relates to the amorphous HBr salt form of Embodiment 53 characterized by one or more of the following:
(a) a powder X-ray diffraction pattern substantially as shown in FIG. 4 using an X-ray wavelength of 1.54 Å;
(b) a modulated differential scanning calorimetry thermogram comprising a glass transition temperature of about 65±1° C.; or
(c) a thermogravimetric analysis thermogram comprising a weight loss of about 0.5%±0.1% when heated from about 25° C. to about 150° C.

Embodiment 58 of this disclosure relates to the amorphous HBr salt form of Embodiment 57, wherein the amorphous HBr salt form is substantially free of other crystalline or amorphous forms.

Embodiment 59 of this disclosure relates to the amorphous HBr salt form of Embodiment 58, wherein the amount of other crystalline or amorphous forms is 5% (w/w) or less.

Embodiment 60 of this disclosure relates to a pharmaceutical composition comprising the amorphous salt form of Compound A of any one of Embodiments 53-59 and a pharmaceutically acceptable excipient.

Embodiment 61 of this disclosure relates to a method of treating a disease mediated by C5aR in a subject in need of treatment, the method comprising administering to the subject a therapeutically effective amount of the amorphous salt form of Compound A of any one of Embodiments 53-59, or the pharmaceutical composition of Embodiment 60.

Embodiment 62 of this disclosure relates to the method of Embodiment 61, wherein the disease mediated by C5aR is anti-neutrophil cytoplasmic autoantibody-associated vasculitis, complement 3 glomerulopathy, hidradenitis suppurativa, or lupus nephritis, or any combination of the foregoing.

Embodiment 63 of this disclosure relates to the method of Embodiment 62, wherein the disease mediated by C5aR is anti-neutrophil cytoplasmic autoantibody-associated vasculitis.

Embodiment 64 of this disclosure relates to the method of any one of Embodiments 61-63, wherein the compound is administered at a total daily dose of EQ 60 mg free base of Compound A or EQ 30 mg freebase of compound A twice daily.

Embodiment 64(a) of this disclosure relates to any one of Embodiments 61-64, further comprising simultaneous, separate, or sequential administration of an effective amount of a second compound, wherein the second compound is one or more anti-inflammatory and/or immunosuppressive agents. Non-limiting examples of the second compound include prednisone, rituximab, cyclophosphamide, or any combination of the foregoing.

Embodiment 65 of this disclosure relates to the crystalline HCl salt form of Embodiment 1 or 2, wherein the crystalline HCl salt form is characterized by a powder X-ray diffraction pattern comprising at least one peak selected from 3.9, 10.5, 12.3, 12.6, 13.9, 15.6, 18.1, 18.6, 21.5, and 25.2±0.2 degrees 2 theta as measured by X-ray powder diffraction using an X-ray wavelength of 1.54 Å.

Embodiment 66 of this disclosure relates to the crystalline HCl salt form of Embodiment 1 or 2, wherein the crystalline HCl salt form is characterized by a powder X-ray diffraction pattern comprising at least two peaks selected from 3.9, 10.5, 12.3, 12.6, 13.9, 15.6, 18.1, 18.6, 21.5, and 25.2±0.2 degrees 2 theta as measured by X-ray powder diffraction using an X-ray wavelength of 1.54 Å.

Embodiment 67 of this disclosure relates to the crystalline HCl salt form of Embodiment 1 or 2, wherein the crystalline HCl salt form is characterized by a powder X-ray diffraction pattern comprising at least three peaks selected from 3.9, 10.5, 12.3, 12.6, 13.9, 15.6, 18.1, 18.6, 21.5, and 25.2±0.2 degrees 2 theta as measured by X-ray powder diffraction using an X-ray wavelength of 1.54 Å.

Embodiment 68 of this disclosure relates to the crystalline HCl salt form of Embodiment 1 or 2, wherein the crystalline HCl salt form is characterized by a powder X-ray diffraction pattern comprising peaks at 3.9, 10.5, 12.3, 12.6, 13.9, 15.6, 18.1, 18.6, 21.5, and 25.2±0.2 degrees 2 theta as measured by X-ray powder diffraction using an X-ray wavelength of 1.54 Å.

Embodiment 69 of this disclosure relates to the crystalline HCl salt form of Embodiment 1 or 2, wherein the crystalline HCl salt form is characterized by a powder X-ray diffraction pattern comprising at least three peaks selected from 3.9, 5.2, 7.7, 10.5, 11.0, 11.7, 12.3, 12.6, 13.9, 14.6, 15.0, 15.6, 17.1, 18.1, 18.6, 19.6, 20.2, 21.5, 21.9, 22.9, 24.5, 25.2, 27.1, 27.9, 29.4, and 35.7±0.2 degrees 2 theta as measured by X-ray powder diffraction using an X-ray wavelength of 1.54 Å.

Embodiment 70 of this disclosure relates to the crystalline HCl salt form of Embodiment 1 or 2, wherein the crystalline HCl salt form is characterized by a powder X-ray diffraction pattern comprising at least five peaks selected from 3.9, 5.2, 7.7, 10.5, 11.0, 11.7, 12.3, 12.6, 13.9, 14.6, 15.0, 15.6, 17.1, 18.1, 18.6, 19.6, 20.2, 21.5, 21.9, 22.9, 24.5, 25.2, 27.1, 27.9, 29.4, and 35.7±0.2 degrees 2 theta as measured by X-ray powder diffraction using an X-ray wavelength of 1.54 Å.

Embodiment 71 of this disclosure relates to the crystalline HCl salt form of Embodiment 1 or 2, wherein the crystalline HCl salt form is characterized by a powder X-ray diffraction pattern comprising at least seven peaks selected from 3.9, 5.2, 7.7, 10.5, 11.0, 11.7, 12.3, 12.6, 13.9, 14.6, 15.0, 15.6, 17.1, 18.1, 18.6, 19.6, 20.2, 21.5, 21.9, 22.9, 24.5, 25.2, 27.1, 27.9, 29.4, and 35.7±0.2 degrees 2 theta as measured by X-ray powder diffraction using an X-ray wavelength of 1.54 Å.

Embodiment 72 of this disclosure relates to the crystalline HCl salt form of Embodiment 1 or 2, wherein the crystalline HCl salt form is characterized by a powder X-ray diffraction pattern comprising peaks at 3.9, 5.2, 7.7, 10.5, 11.0, 11.7, 12.3, 12.6, 13.9, 14.6, 15.0, 15.6, 17.1, 18.1, 18.6, 19.6, 20.2, 21.5, 21.9, 22.9, 24.5, 25.2, 27.1, 27.9, 29.4, and 35.7±0.2 degrees 2 theta as measured by X-ray powder diffraction using an X-ray wavelength of 1.54 Å.

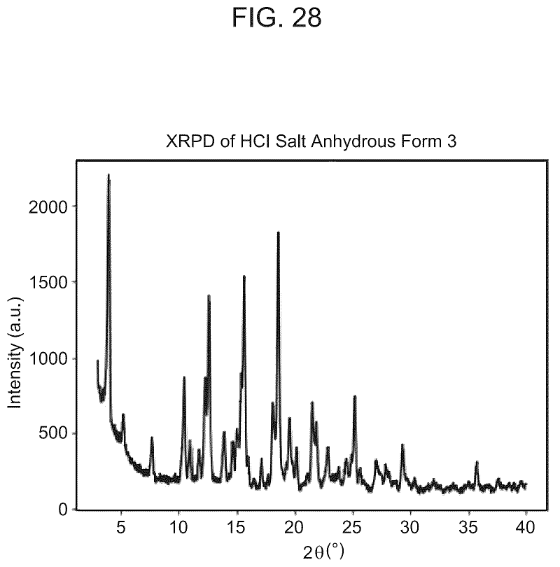

Embodiment 73 of this disclosure relates to the crystalline HCl salt form of Embodiment 1 or 2, wherein the crystalline HCl salt form is characterized by the powder X-ray diffraction pattern substantially as shown in FIG. 28 as measured by X-ray powder diffraction using an X-ray wavelength of 1.54 Å.

Embodiment 74 of this disclosure relates to the crystalline HCl salt form of any one of Embodiments 1, 2 or 65-73, wherein the crystalline HCl salt form is characterized by a differential scanning calorimetry thermogram comprising an endotherm with an onset of about 209±1° C.

Embodiment 75 of this disclosure relates to the crystalline HCl salt form of any one of Embodiments 1, 2 or 65-74, wherein the crystalline HCl salt is characterized by a thermogravimetric analysis thermogram comprising a weight loss of about 0.2%±0.1% when heated from about 25° C. to about 150° C.

Embodiment 76 of this disclosure relates to the crystalline HCl salt form of any one of Embodiments 1, 2 or 65-74, wherein the crystalline HCl salt is characterized by a thermogravimetric analysis thermogram comprising a weight loss of about 0.2%±0.05% when heated from about 25° C. to about 150° C.

Embodiment 77 of this disclosure relates to the crystalline HCl salt of any one of Embodiments 1, 2 or 65-76, wherein the crystalline HCl salt form is characterized by ¹³C solid state NMR comprising at least three peaks selected from peaks at approximately 170.2, 166.4, 158.9, 158.0, 157.3, 139.8, 138.1, 136.6, 134.9, 132.7, 131.6, 130.5, 128.4, 127.0, 126.5, 123.5, 122.4, 119.6, 114.9, 113.6, 65.0, 63.3, 56.4, 52.4, 47.7, 46.9, 42.9, 30.1, 25.8, 25.4, 22.9, and 19.3.

Embodiment 78 of this disclosure relates to the crystalline HCl salt of any one of Embodiments 1, 2 or 65-76, wherein the crystalline HCl salt form is characterized by ¹³C solid state NMR comprising at least five peaks selected from peaks at approximately 170.2, 166.4, 158.9, 158.0, 157.3, 139.8, 138.1, 136.6, 134.9, 132.7, 131.6, 130.5, 128.4, 127.0, 126.5, 123.5, 122.4, 119.6, 114.9, 113.6, 65.0, 63.3, 56.4, 52.4, 47.7, 46.9, 42.9, 30.1, 25.8, 25.4, 22.9, and 19.3.

Embodiment 79 of this disclosure relates to the crystalline HCl salt of any one of Embodiments 1, 2 or 65-76, wherein the crystalline HCl salt form is characterized by ¹³C solid state NM R comprising at least seven peaks selected from peaks at approximately 170.2, 166.4, 158.9, 158.0, 157.3, 139.8, 138.1, 136.6, 134.9, 132.7, 131.6, 130.5, 128.4, 127.0, 126.5, 123.5, 122.4, 119.6, 114.9, 113.6, 65.0, 63.3, 56.4, 52.4, 47.7, 46.9, 42.9, 30.1, 25.8, 25.4, 22.9, and 19.3 ppm.

Embodiment 80 of this disclosure relates to the crystalline HCl salt of any one of Embodiments 1, 2 or 65-76, wherein the crystalline HCl salt form is characterized by ¹³C solid state NM R comprising peaks at approximately 170.2, 166.4, 158.9, 158.0, 157.3, 139.8, 138.1, 136.6, 134.9, 132.7, 131.6, 130.5, 128.4, 127.0, 126.5, 123.5, 122.4, 119.6, 114.9, 113.6, 65.0, 63.3, 56.4, 52.4, 47.7, 46.9, 42.9, 30.1, 25.8, 25.4, 22.9, and 19.3 ppm.

Embodiment 81 of this disclosure relates to the crystalline HCl salt form of any one of Embodiments 1, 2 or 65-79, wherein the crystalline HCl salt form is substantially free of other crystalline or amorphous forms.

Embodiment 82 of this disclosure relates to the crystalline HCl salt form of Embodiment 1, wherein the crystalline HCl salt form is a TH F/H₂O solvate.

Embodiment 83 of this disclosure relates to the crystalline THF/H₂O solvate of Embodiment 82, wherein the crystalline TH F/H₂O solvate is characterized by a powder X-ray diffraction pattern comprising at least three peaks selected from 9.4, 13.4, 14.7, 15.7, 17.0, 17.8, 18.9, 19.5, 20.8, 22.6, 23.6, 25.0, 26.8, 27.2, 28.3, 29.0, 31.3, 34.5, 36.2, and 40.6±0.2 degrees 2 theta as measured by X-ray powder diffraction using an X-ray wavelength of 1.54 Å.

Embodiment 84 of this disclosure relates to the crystalline THF/H₂O solvate of Embodiment 83, further characterized by one or more of the following:
a. one or more peaks selected from 9.4, 15.7, 17.0, 17.8, 20.8, 27.2, 28.3, 29.0, 31.3, 34.5, 36.2, and 40.6±0.2 degrees 2 theta as measured by X-ray powder diffraction using an X-ray wavelength of 1.54 Å;
b. a differential scanning calorimetry thermogram comprising an endotherm with an onset of about 117±1° C.; or
c. a thermogravimetric analysis thermogram comprising a weight loss of about 9%±1% when heated from about 25° C. to about 100° C.

Embodiment 85 of this disclosure relates to the crystalline TH F/H₂O solvate form of Embodiment 84, wherein the amount of other crystalline or amorphous forms is 5% (w/w) or less.

Embodiment 86 of this disclosure relates to the crystalline HCl salt form of Embodiment 1, wherein the crystalline HCl salt form is characterized by one or more of the following:
(a) a powder X-ray diffraction pattern comprising at least one peak selected from 5.9, 7.3, 13.4, 13.7, 15.4, 16.3, 17.7, 18.7, 21.0, and 22.4±0.2 degrees 2 theta as measured by X-ray powder diffraction using an X-ray wavelength of 1.54 Å;
(b) a differential scanning calorimetry thermogram comprising an endotherm with an onset of about 192±1° C.;
(c) a thermogravimetric analysis thermogram comprising a weight loss of about 0.33%±0.1% when heated from about 25° C. to about 125° C.; and
(d) ¹³C solid state NMR comprising at least three peaks selected from peaks at approximately 170, 166, 160, 158, 140, 137, 135, 132, 129, 126, 124, 116, 115, 111, 65, 60, 54, 52, 49, 46, 44, 30, 28, 25, and 19 ppm.

Embodiment 86(a) of this disclosure relates to the crystalline HCl salt form of Embodiment 86, wherein the crystalline HCl salt form is characterized by one or more of the following:
(a) a powder X-ray diffraction pattern comprising at least one peak selected from 5.9, 7.3, 13.4, 13.7, 15.4, 16.3, 17.7, 18.7, 21.0, and 22.4±0.1 degrees 2 theta as measured by X-ray powder diffraction using an X-ray wavelength of 1.54 Å;
(b) a differential scanning calorimetry thermogram comprising an endotherm with an onset of about 192±1° C.;
(c) a thermogravimetric analysis thermogram comprising a weight loss of about 0.33%±0.1% when heated from about 25° C. to about 125° C.; and
(d) ¹³C solid state NMR comprising at least three peaks selected from peaks at approximately 170, 166, 160, 158, 140, 137, 135, 132, 129, 126, 124, 116, 115, 111, 65, 60, 54, 52, 49, 46, 44, 30, 28, 25, and 19 ppm.

Embodiment 86(b) of this disclosure relates to the crystalline HCl salt form of Embodiment 86, wherein the powder X-ray diffraction pattern comprises peaks at 13.7, 15.4 and 16.3±0.2 degrees 2 theta as measured by X-ray powder diffraction using an X-ray wavelength of 1.54 Å.

Embodiment 86(c) of this disclosure relates to the crystalline HCl salt form of Embodiment 86, wherein the powder X-ray diffraction pattern comprises peaks at 13.7, 15.4 and 16.3±0.1 degrees 2 theta as measured by X-ray powder diffraction using an X-ray wavelength of 1.54 Å.

Embodiment 87 of this disclosure relates to the crystalline HCl salt form of Embodiment 86, wherein the X-ray diffraction pattern comprising at least two peaks selected from 5.9, 7.3, 13.4, 13.7, 15.4, 16.3, 17.7, 18.7, 21.0, and 22.4±0.2 degrees 2 theta as measured by X-ray powder diffraction using an X-ray wavelength of 1.54 Å.

Embodiment 87(a) of this disclosure relates to the crystalline HCl salt form of Embodiment 86, wherein the X-ray diffraction pattern comprising at least two peaks selected from 5.9, 7.3, 13.4, 13.7, 15.4, 16.3, 17.7, 18.7, 21.0, and 22.4±0.1 degrees 2 theta as measured by X-ray powder diffraction using an X-ray wavelength of 1.54 Å.

Embodiment 88 of this disclosure relates to the crystalline HCl salt form of Embodiment 86, wherein the crystalline HCl salt form is characterized by a powder X-ray diffraction pattern comprises at least three peaks selected from 5.9, 7.3, 13.4, 13.7, 15.4, 16.3, 17.7, 18.7, 21.0, and 22.4±0.2 degrees 2 theta as measured by X-ray powder diffraction using an X-ray wavelength of 1.54 Å.

Embodiment 88(a) of this disclosure relates to the crystalline HCl salt form of Embodiment 86, wherein the crystalline HCl salt form is characterized by a powder X-ray diffraction pattern comprises at least three peaks selected from 5.9, 7.3, 13.4, 13.7, 15.4, 16.3, 17.7, 18.7, 21.0, and 22.4±0.1 degrees 2 theta as measured by X-ray powder diffraction using an X-ray wavelength of 1.54 Å.

Embodiment 89 of this disclosure relates to the crystalline HCl salt form of Embodiment 86, wherein the powder X-ray diffraction pattern comprises peaks at 5.9, 7.3, 13.4, 13.7, 15.4, 16.3, 17.7, 18.7, 21.0, and 22.4±0.2 degrees 2 theta as measured by X-ray powder diffraction using an X-ray wavelength of 1.54 Å.

Embodiment 89(a) of this disclosure relates to the crystalline HCl salt form of Embodiment 89, wherein the powder X-ray diffraction pattern comprises peaks at 5.9, 7.3, 13.4, 13.7, 15.4, 16.3, 17.7, 18.7, 21.0, and 22.4±0.1 degrees 2 theta as measured by X-ray powder diffraction using an X-ray wavelength of 1.54 Å.

Embodiment 90 of this disclosure relates to the crystalline HCl salt form of any one of Embodiments 86-89, wherein the powder X-ray diffraction pattern further comprises at least one peak selected from 6.7, 7.3, 10.3, 11.5, 12.9, 13.4, 19.8, 21.9, 23.1, 23.8, 25.7, and 28.3±0.2 degrees 2 theta as measured by powder X-ray diffraction using an X-ray wavelength of 1.54 Å.

Embodiment 90(a) of this disclosure relates to the crystalline HCl salt form of any one of Embodiments 90, wherein the powder X-ray diffraction pattern further comprises at least one peak selected from 6.7, 7.3, 10.3, 11.5, 12.9, 13.4, 19.8, 21.9, 23.1, 23.8, 25.7, and 28.3±0.1 degrees 2 theta as measured by powder X-ray diffraction using an X-ray wavelength of 1.54 Å.

Embodiment 91 relates to the crystalline HCl salt form of Embodiment 70, wherein the powder X-ray diffraction pattern comprises peaks at 5.9, 6.7, 7.3, 10.3, 11.5, 12.9, 13.4, 13.7, 15.4, 16.3, 17.7, 18.7, 19.8, 21.0, 21.9, 22.4, 23.1, 23.8, 25.7, and 28.3±0.2 degrees 2 theta as measured by X-ray powder diffraction using an X-ray wavelength of 1.54 Å.

Embodiment 91(a) relates to the crystalline HCl salt form of Embodiment 91, wherein the powder X-ray diffraction pattern comprises peaks at 5.9, 6.7, 7.3, 10.3, 11.5, 12.9, 13.4, 13.7, 15.4, 16.3, 17.7, 18.7, 19.8, 21.0, 21.9, 22.4, 23.1, 23.8, 25.7, and 28.3±0.1 degrees 2 theta as measured by X-ray powder diffraction using an X-ray wavelength of 1.54 Å.

Embodiment 92 relates to the crystalline HCl salt form of Embodiment 86, wherein the crystal powder X-ray diffraction pattern is substantially as shown in FIG. 7 as measured by X-ray powder diffraction using an X-ray wavelength of 1.54 Å.

Embodiment 93 relates to the crystalline HCl salt form of any one of Embodiments 86-92, or any sub-embodiments thereof, wherein the thermogravimetric analysis thermogram further comprising a weight loss of about 5.6%±0.5% from about 125° C. to about 200° C.

Embodiment 94 relates to the crystalline HCl salt of any one of Embodiments 86-93, or any sub-embodiments thereof, wherein the ¹³C solid state NMR comprises at least five peaks selected from peaks at approximately 170, 166, 160, 158, 140, 137, 135, 132, 129, 126, 124, 116, 115, 111, 65, 60, 54, 52, 49, 46, 44, 30, 28, 25, and 19 ppm.

Embodiment 95 relates to the crystalline HCl salt of any one of Embodiments 86-93, or any sub-embodiments thereof, wherein the ¹³C solid state NMR comprises at least seven peaks selected from peaks at approximately 170, 166, 160, 158, 140, 137, 135, 132, 129, 126, 124, 116, 115, 111, 65, 60, 54, 52, 49, 46, 44, 30, 28, 25, and 19 ppm.

Embodiment 96 relates to the crystalline HCl salt of any one of Embodiments 86-93 or any sub-embodiments thereof, wherein the ¹³C solid state NM R comprises peaks at approximately 170, 166, 160, 158, 140, 137, 135, 132, 129, 126, 124, 116, 115, 111, 65, 60, 54, 52, 49, 46, 44, 30, 28, 25, and 19 ppm.

Embodiment 97 relates to the crystalline HCl salt form of any one of Embodiments 86-96, or any sub-embodiments thereof, wherein the crystalline HCl salt form is substantially free of other crystalline or amorphous forms.

Embodiment 97(a) relates to the crystalline HCl salt form of Embodiment 97, wherein the amount of other crystalline or amorphous forms is 5% (w/w) or less.

Embodiment 98 of this disclosure relates to the crystalline HBr salt form of Embodiment 2, wherein the crystalline HBr salt form is characterized by one or more of the following:
(a) at least one peak selected from 7.5, 12.8, 13.2, 13.6, 15.3, 17.9, 18.8, 20.9, 21.3, 22.9, and 25.2±0.2 degrees 2 theta as measured by X-ray powder diffraction using an X-ray wavelength of 1.54 Å;
(b) a differential scanning calorimetry thermogram comprising an endotherm with an onset of about 201±1° C.;
(c) a thermogravimetric analysis thermogram comprising a weight loss of about 0.35%±0.1% when heated from about 25° C. to about 150° C.; and
(d) ¹³C solid state NMR comprising at least three peaks selected from peaks at approximately 170, 166, 160, 159, 158, 157, 141, 137, 135, 132, 129, 126, 124, 117, 115, 111, 65, 60, 52, 46, 44, 30, 28, 24, and 19 ppm.

Embodiment 98(a) of this disclosure relates to the crystalline HBr salt form of Embodiment 1, wherein the crystalline HBr salt form is characterized by one or more of the following:
(a) at least one peak selected from 7.5, 12.8, 13.2, 13.6, 15.3, 17.9, 18.8, 20.9, 21.3, 22.9, and 25.2±0.1 degrees 2 theta as measured by X-ray powder diffraction using an X-ray wavelength of 1.54 Å;
(b) a differential scanning calorimetry thermogram comprising an endotherm with an onset of about 201±1° C.;
(c) a thermogravimetric analysis thermogram comprising a weight loss of about 0.35%±0.1% when heated from about 25° C. to about 150° C.; and
(d) ¹³C solid state NMR comprising at least three peaks selected from peaks at approximately 170, 166, 160, 159, 158, 157, 141, 137, 135, 132, 129, 126, 124, 117, 115, 111, 65, 60, 52, 46, 44, 30, 28, 24, and 19 ppm.
   is characterized by a powder X-ray diffraction pattern comprising peaks at 13.2, 15.3 and 20.9±0.2 degrees 2 theta as measured by X-ray powder diffraction using an X-ray wavelength of 1.54 Å.

Embodiment 98(b) of this disclosure relates to Embodiment 98 wherein the powder X-ray diffraction pattern comprises peaks at 13.2, 15.3 and 20.9±0.2 degrees 2 theta as measured by X-ray powder diffraction using an X-ray wavelength of 1.54 Å.

Embodiment 98(c) of this disclosure relates to Embodiment 98 wherein the powder X-ray diffraction pattern comprises peaks at 13.2, 15.3 and 20.9±0.1 degrees 2 theta as measured by X-ray powder diffraction using an X-ray wavelength of 1.54 Å.

Embodiment 99 of this disclosure relates to the crystalline HBr salt form of Embodiment 98, wherein the powder X-ray diffraction pattern comprises at least two peaks selected from 7.5, 12.8, 13.2, 13.6, 15.3, 17.9, 18.8, 20.9, 21.3, 22.9, and 25.2±0.2 degrees 2 theta as measured by X-ray powder diffraction using an X-ray wavelength of 1.54 Å.

Embodiment 99(a) of this disclosure relates to the crystalline HBr salt form of Embodiment 99, wherein the powder X-ray diffraction pattern comprises at least two peaks selected from 7.5, 12.8, 13.2, 13.6, 15.3, 17.9, 18.8, 20.9, 21.3, 22.9, and 25.2±0.1 degrees 2 theta as measured by X-ray powder diffraction using an X-ray wavelength of 1.54 Å.

Embodiment 100 of this disclosure relates to the crystalline HBr salt form of Embodiment 98, wherein the powder X-ray diffraction pattern comprises at least three peaks selected from 7.5, 12.8, 13.2, 13.6, 15.3, 17.9, 18.8, 20.9, 21.3, 22.9, and 25.2±0.2 degrees 2 theta as measured by X-ray powder diffraction using an X-ray wavelength of 1.54 Å.

Embodiment 100(a) of this disclosure relates to the crystalline HBr salt form of Embodiment 100, wherein the powder X-ray diffraction pattern comprises at least three peaks selected from 7.5, 12.8, 13.2, 13.6, 15.3, 17.9, 18.8, 20.9, 21.3, 22.9, and 25.2±0.1 degrees 2 theta as measured by X-ray powder diffraction using an X-ray wavelength of 1.54 Å.

Embodiment 101 of this disclosure relates to the crystalline HBr salt form of Embodiment 98, wherein the powder X-ray diffraction pattern comprises peaks at 7.5, 12.8, 13.2, 13.6, 15.3, 17.9, 18.8, 20.9, 21.3, 22.9, and 25.2±0.2 degrees 2 theta as measured by X-ray powder diffraction using an X-ray wavelength of 1.54 Å.

Embodiment 101(a) of this disclosure relates to the crystalline HBr salt form of Embodiment 101, wherein the powder X-ray diffraction pattern comprises peaks at 7.5, 12.8, 13.2, 13.6, 15.3, 17.9, 18.8, 20.9, 21.3, 22.9, and 25.2±0.1 degrees 2 theta as measured by X-ray powder diffraction using an X-ray wavelength of 1.54 Å.

Embodiment 102 of this disclosure relates to the crystalline HBr salt form of any of Embodiments 98-101, or any sub-embodiments thereof, wherein the powder X-ray diffraction pattern further comprises at least one peak selected from 10.1, 11.8, 12.5, 16.0, 16.4, 17.4, 19.8, 20.3, 22.0, 23.3, 23.8, 24.7, 26.4, 27.8, 28.9, 30.0, 32.8, and 34.7±0.2 degrees 2 theta as measured by X-ray powder diffraction using an X-ray wavelength of 1.54 Å.

Embodiment 102(a) of this disclosure relates to the crystalline HBr salt form of any of Embodiments 102, wherein the powder X-ray diffraction pattern further comprises at least one peak selected from 10.1, 11.8, 12.5, 16.0, 16.4, 17.4, 19.8, 20.3, 22.0, 23.3, 23.8, 24.7, 26.4, 27.8, 28.9, 30.0, 32.8, and 34.7±0.1 degrees 2 theta as measured by X-ray powder diffraction using an X-ray wavelength of 1.54 Å.

Embodiment 103 of this disclosure relates to the crystalline HBr salt form of Embodiment 98, wherein the powder X-ray diffraction pattern is substantially as shown in FIG. 11 as measured by X-ray powder diffraction using an X-ray wavelength of 1.54 Å.

Embodiment 104 of this disclosure relates to the crystalline HBr salt of any one of Embodiments 98-103, or any sub-embodiments thereof, wherein the ¹³C solid state NM R comprises at least five peaks selected from peaks at approximately 170, 166, 160, 159, 158, 157, 141, 137, 135, 132, 129, 126, 124, 117, 115, 111, 65, 60, 52, 46, 44, 30, 28, 24, and 19 ppm.

Embodiment 105 of this disclosure relates to the crystalline HBr salt of any one of Embodiments 98-103, or any sub-embodiments thereof, wherein the ¹³C solid state NM R comprises at least seven peaks selected from peaks at approximately 170, 166, 160, 159, 158, 157, 141, 137, 135, 132, 129, 126, 124, 117, 115, 111, 65, 60, 52, 46, 44, 30, 28, 24, and 19 ppm.

Embodiment 106 of this disclosure relates to the crystalline HBr salt of any one of Embodiments 98-103, or any sub-embodiments thereof, wherein the ¹³C solid state NM R comprises peaks at approximately 170, 166, 160, 159, 158, 157, 141, 137, 135, 132, 129, 126, 124, 117, 115, 111, 65, 60, 52, 46, 44, 30, 28, 24, and 19 ppm.

Embodiment 107 of this disclosure relates to the crystalline HBr salt form of any one of Embodiments 98-106, or any sub-embodiments thereof, wherein the crystalline HBr salt form is substantially free of other crystalline or amorphous forms.

Embodiment 108 of this disclosure relates to the crystalline HBr salt form of Embodiment 107, wherein the amount of other crystalline or amorphous forms is 5% (w/w) or less.

Embodiment 109 of this disclosure relates to a pharmaceutical composition comprising the crystalline salt form of Compound A of any one of Embodiments 86-108, or any sub-embodiments thereof, and a pharmaceutically acceptable excipient.

Embodiment 110 of this disclosure relates to a method of treating a disease mediated by C5aR in a subject in need of treatment, the method comprising administering to the subject a therapeutically effective amount of the crystalline salt form of Compound A of any one of Embodiments 86-108, or any sub-embodiments thereof, or the pharmaceutical composition of Embodiment 109.

Embodiment 111 of this disclosure relates to the method of Embodiment 110, wherein the disease mediated by C5aR is anti-neutrophil cytoplasmic autoantibody-associated vasculitis, complement 3 glomerulopathy, hidradenitis suppurativa, or lupus nephritis, or any combination of the foregoing.

Embodiment 112 of this disclosure relates to a method of Embodiment 111, wherein the disease mediated by C5aR is anti-neutrophil cytoplasmic autoantibody-associated vasculitis.

Embodiment 113 of this disclosure relates to the method of any one of Embodiments 110-112, wherein the compound is administered at a total daily dose of EQ 60 mg free base of Compound A or EQ 30 mg freebase of compound A twice daily.

Embodiment 114 of this disclosure relates to the crystalline salt form of Compound A of any one of Embodiments 1-39, or any sub-embodiments thereof, or the pharmaceutical composition of Embodiment 40, for use as a medicament for the treatment of a disease mediated by C5aR in a subject in need of treatment.

Embodiment 115 of this disclosure relates Embodiment 114, wherein the disease mediated by C5aR is anti-neutrophil cytoplasmic autoantibody-associated vasculitis, complement 3 glomerulopathy, hidradenitis suppurativa, or lupus nephritis, or any combination of the foregoing.

Embodiment 116 of this disclosure relates to a method of Embodiment 115, wherein the disease mediated by C5aR is anti-neutrophil cytoplasmic autoantibody-associated vasculitis.

Embodiment 117 of this disclosure relates to any one of Embodiments 114-116, wherein the compound is administered at a total daily dose of EQ 60 mg free base of Compound A or EQ 30 mg freebase of compound A twice daily.

Embodiment 118 of this disclosure relates to the crystalline salt form of Compound A of any one of Embodiments 1-39, or any sub-embodiments thereof, or the pharmaceutical composition of Embodiment 40, for use as a medicament for the treatment of a disease mediated by C5aR in a subject in need of treatment.

Embodiment 119 of this disclosure relates to Embodiment 118, wherein the disease mediated by C5aR is anti-neutrophil cytoplasmic autoantibody-associated vasculitis, complement 3 glomerulopathy, hidradenitis suppurativa, or lupus nephritis, or any combination of the foregoing.

Embodiment 120 of this disclosure relates to Embodiment 119, wherein the disease mediated by C5aR is anti-neutrophil cytoplasmic autoantibody-associated vasculitis.

Embodiment 121 of this disclosure relates to any one of Embodiments 118-120, wherein the compound is administered at a total daily dose of EQ 60 mg free base of Compound A or EQ 30 mg freebase of compound A twice daily.

Embodiment 122 of this disclosure relates to the crystalline salt form of Compound A of any one of Embodiments 1-39, or any sub-embodiments thereof, or the pharmaceutical composition of Embodiment 40, for use in the treatment of a disease mediated by C5aR in a subject in need of treatment.

Embodiment 123 of this disclosure relates to Embodiment 122, wherein the disease mediated by C5aR is anti-neutrophil cytoplasmic autoantibody-associated vasculitis, complement 3 glomerulopathy, hidradenitis suppurativa, or lupus nephritis, or any combination of the foregoing.

Embodiment 124 of this disclosure relates to Embodiment 123, wherein the disease mediated by C5aR is anti-neutrophil cytoplasmic autoantibody-associated vasculitis.

Embodiment 125 of this disclosure relates to any one of Embodiments 122-124, wherein the compound is administered at a total daily dose of EQ 60 mg free base of Compound A or EQ 30 mg freebase of compound A twice daily.

Embodiment 126 of this disclosure relates to the crystalline salt form of Compound A of any one of Embodiments 1-39, or any sub-embodiments thereof, or the pharmaceutical composition of Embodiment 40, for the manufacture of a medicament for the treatment of a disease mediated by C5aR in a subject in need of treatment.

Embodiment 127 of this disclosure relates to Embodiment 126, wherein the disease mediated by C5aR is anti-neutrophil cytoplasmic autoantibody-associated vasculitis, complement 3 glomerulopathy, hidradenitis suppurativa, or lupus nephritis, or any combination of the foregoing.

Embodiment 128 of this disclosure relates to Embodiment 127, wherein the disease mediated by C5aR is anti-neutrophil cytoplasmic autoantibody-associated vasculitis.

Embodiment 129 of this disclosure relates to any one of Embodiments 126-128, wherein the compound is administered at a total daily dose of EQ 60 mg free base of Compound A or EQ 30 mg freebase of compound A twice daily.

Embodiment 130 of this disclosure relates to a process of making crystalline HCl salt form of Compound A according to any one of Embodiments 2-20, or Embodiments 86-98, or any sub-embodiments thereof, comprising:
providing a free base form of Compound A;
combining the free base form of Compound A with toluene under heated conditions; adding an aqueous acid; and
isolating the crystalline HCl salt form of Compound A.

Embodiment 131 of this disclosure relates to the process of Embodiment 130, wherein the aqueous acid is aqueous HCl.

Embodiment 132 of this disclosure relates to the process of any one of Embodiments 130-131, wherein the heated conditions for combining the free base form of Compound A with toluene comprises a temperature ranging from about 50° C. to about 70° C.

Embodiment 133 of this disclosure relates to the process of any one of Embodiments 130-131, wherein the heated conditions for combining the free base form of Compound A with toluene comprises a temperature ranging from about 55° C. to about 65° C.

Embodiment 134 of this disclosure relates to the process of any one of Embodiments 130-131, wherein the heated conditions for combining the free base form of Compound A with toluene comprises heating to a temperature of about 62° C.

Embodiment 135 of this disclosure relates to the process of any one of Embodiments 130-134, wherein the isolation of crystalline HCl salt form of Compound A comprises vacuum drying under heated condition.

Embodiment 136 of this disclosure relates to the process of Embodiment 135, wherein the heated conditions comprises a temperature ranging from about 50° C. to about 70° C.

Embodiment 137 of this disclosure relates to the process of Embodiment 135, wherein the heated conditions comprises a temperature ranging from about 55° C. to about 65° C.

Embodiment 138 of this disclosure relates to the process of Embodiment 135, wherein the heated conditions comprises a temperature of about 60° C.

Embodiment 139 of this disclosure relates to a process of making process of making crystalline HBr salt form of Compound A according to any one of Embodiments 21-39, or Embodiments 98-108, or any sub-embodiments thereof, comprising:
providing free base amorphous form of Compound A;
combining the free base amorphous form of Compound with acetonitrile under heated conditions;
isolating the precipitate from the reaction mixture; and
washing and drying the isolated precipitate to afford crystalline HBr salt form of Compound A.

Embodiment 140 of this disclosure relates to the process of Embodiment 139, wherein the heated conditions for combining the free base amorphous form of Compound with acetonitrile comprises a temperature ranging from about 50° C. to about 70° C.,

Embodiment 141 of this disclosure relates to the process of Embodiment 139, wherein the heated conditions for combining the free base amorphous form of Compound with acetonitrile comprises a temperature ranging from about 55° C. to about 65° C.

Embodiment 142 of this disclosure relates to the process of Embodiment 139, wherein the heated conditions for combining the free base amorphous form of Compound with acetonitrile comprises a temperature of about 60° C.

Embodiment 143 of this disclosure relates to the process of any one of Embodiments 139-142, wherein the isolation of crystalline HBr salt form of Compound A comprises filtering and optionally filter re-slurrying.

Embodiment 144 of this disclosure relates to the process of any one of Embodiments 139-143, wherein the washing and drying steps are performed under heated conditions.

Embodiment 145 of this disclosure relates to the process of Embodiments 144, wherein the heated conditions comprises a temperature ranging from about 110° C. to about 140° C.

Embodiment 146 of this disclosure relates to the process of Embodiment 145, wherein the heated conditions comprises a temperature ranging from about 120° C. to about 130° C.

Embodiment 147 of this disclosure relates to the process of Embodiment 145, wherein the heated conditions comprises a temperature of about 125° C.

### Analytical Techniques

### Powder X-Ray Diffraction (PXRD)

Powder X-ray diffraction data were collected using a Bruker D8 Advance diffractometer equipped with a twin-twin optic and EIGER2 R 500K X-ray detector in reflection mode. The samples were scanned at ambient temperature in continuous mode from 3-40 °2θ with step size of 0.02 °2θ at 40 kV and 40 mA with CuKα radiation (1.54 Å). The incident beam path was equipped with primary soller slit 2.5-degree, secondary soller slit 4.0 and divergence slit 0.6 mm, in a fixed slit mode. Samples were prepared on a low background sample holder and placed on a spinning stage with a rotation time of 10 rev/min. The 20 position was calibrated against a Panalytical Si reference standard disc. Data were collected using DIFFRAC.MEASUREMENT CENTER (v. 7.5) and processed with DIFFRAC.EVA (v. 5.2). The parameters used are listed in the table below.

### Parameters for PXRD

| Parameters | Reflection Mode | |
|---|---|---|
| X-Ray wavelength | Cu, kα | |
| | Kα1 (Å): 1.540598, | |
| | Kα2 (Å): 1.544426, | |
| | Kα2/Kα1 intensity ratio: 0.50 | |
| X-Ray tube setting | 40 kV, 40 mA | |
| Divergence slit | 0.6 | mm |
| Scan mode | Continuous | |
| Scan range (°2θ) | 3-40 | |
| Scan step time (s) | 37.27 | |
| Step size (°2θ) | 0.02 | |
| Test Time | 7 | min |

It is noted that peak shift of about +/−0.2 degrees can occur in XRPD patterns and could be caused by factors such as sample preparation and instrument alignment.

### Thermogravimetric Analysis (TGA)

Thermogravimetric analysis was performed in a TGA Q500 (TA Instruments Inc.) calibrated with calcium oxalate monohydrate. After the pan was tared, the samples (2.00-10.00 mg) were equilibrated at 25° C. prior to heating to 300° C. under N2 atmosphere (25 mL/min) at a rate of 10° C./min in a platinum pan. Data were analyzed with TR IOS v5.4 TA Universal Analysis software version 4.5A. Detailed parameters used are listed in the table below.

### Differential Scanning Calorimetry (DSC)

Differential scanning calorimetry data were collected using a DSC Q2000 (TA Instruments Inc.) equipped with an RCS90 (Discovery) single-stage refrigeration system and auto sampler. The calibration of the instrument was made with an indium standard. Approximately 2.00-10.00 mg of the powder samples were weighed using a XP6 microbalance from MettlerToledo (±0.002 mg) and placed on normal crimped aluminum pans. Samples were equilibrated at 25° C. prior to heating to 300° C. under a N2 atmosphere (50 mL/min) at a rate of 10° C./min and temperature accuracy of 0.1° C. Data were analyzed with TRIOS v5.4 TA Universal Analysis 2000 software version 4.5A.

Modulated DSC runs were collected at a heating rate of 2° C./min with a modulation frequency of 1° C. per 100 s over a temperature range of −40 to 300° C.

Detailed parameters used are listed in the table below.

| Parameters | TGA | DSC |
|---|---|---|
| Method | Ramp | Ramp |
| Sample pan | Platinum, open | Aluminum |
| Temperature | RT - desired temperature | 25° C. - desired temperature |
| Heating rate | 10° C./min | 10° C./min |
| Purge gas | N₂ | N₂ |

### Solid State NMR

All spectra were acquired at 11.7 T on a wide bore Bruker Avance III spectrometer equipped with a 4 mm H/F/X magic angle spinning probe at 298 K. Chemical shifts were referenced externally using adamantane with the field set to that the ¹³C CH₂ resonance was at 38.48 ppm. Approximately 100 mg of sample was packed into a 4 mm zirconia rotor and a magic angle spinning frequency of 10 kHz was used.

¹³C spectra were acquired by ¹H-¹³C cross polarization with total suppression of sidebands sequence (CP-TOSS) using a 70-100% amplitude ramped 1H pulse and a constant amplitude 13C pulse with maximum RF fields at ˜60 kHz and ˜50 kHz respectively. A CP contact time of 2 ms was used. 10 us 13C pi pulses at 50 kHz were employed. During acquisition 1H heteronuclear decoupling was achieved using SPINA L-64. 1944 transients were acquired for each spectrum using a recycle delay of 40 s. A II other acquisition parameters can be found in the relevant data sets.

### Dynamic Vapor Sorption

Dynamic Vapor Sorption (DVS): Water sorption data were collected using a DVS Adventure dynamic vapor sorption analyzer (Surface Measurement Systems). The relative humidity at 25° C. was calibrated against the deliquescence point of LiCl, Mg(NO₃)₂ and KCl. Approximately 5-15 mg were loaded onto a tared metal pan. Data were collected from 0 to 90 to 0% RH in 5% increments at 25° C. Progression to the next stage occurred when the change in mass with respect to time was less than 0.002 per minute or after 180 minutes, whichever occurred first. The minimum stage time was 10 minutes.

Actual parameters for DVS are listed in the table below.

| Item | Value | |
|---|---|---|
| Temperature | 25° | C. |
| Sample size | 5~15 | mg |
| Gas and flow rate | N₂, 200 mL/min | |
| dm/dt | 0.002/min | |
| Min. dm/dt stability duration | 10 | min |
| Max. equilibrium time | 180 | min |

| | |
|---|---|
| RH range | 0% RH-90% RH-0% RH |
| RH step size | 5% RH |

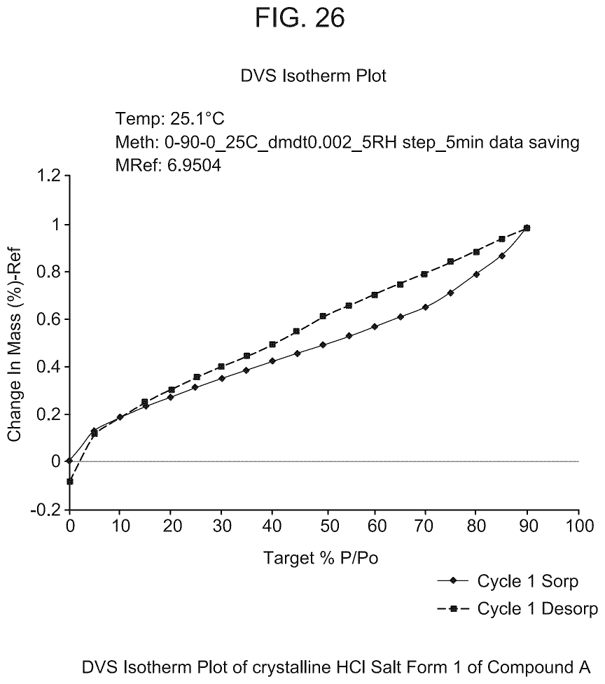

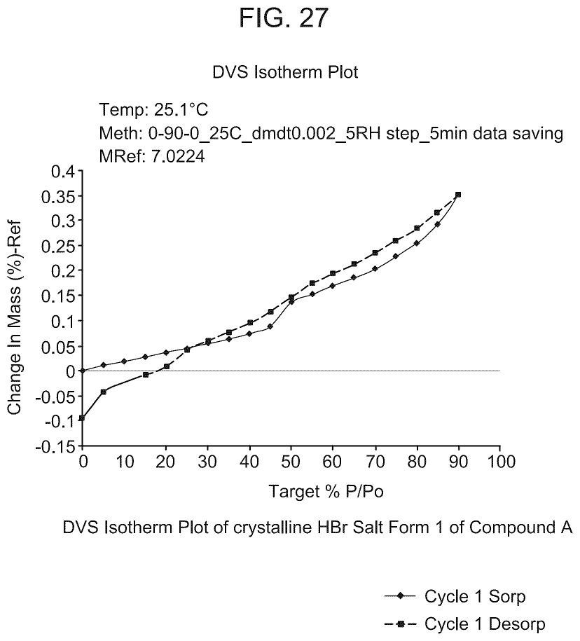

The DVS data in FIGS. 26 and 27 indicate that both the crystalline HCl and HBr salt forms of Compound A are non-hygroscopic.

### Solubility

Solubility: Samples were prepared by adding ˜10 mg of the solid to a small centrifuge tube followed by the addition of 1.75 mL freshly prepared FaSSIF solution. The pH of each suspension was measured prior to shaking the samples in an Eppendorf ThermoMixer F1.5 at 37° C. and 1500 rpm for 2 hr. The samples were centrifuged, and the pH of each suspension was measured again at the T=2 hr timepoint. The samples were filtered through Millex 0.20 μm PTFE syringe filters and the filtrates were diluted by a factor of 2 with acetonitrile. Solubility samples were prepared in duplicate. Calibration standards were prepared at API concentrations of 1, 2.5, 7.5, 15, 25, and 50 μg/mL using 1:1 H2O/ACN as a diluent (linear regression R2=0.999965). A check standard was prepared at a concentration of 14.94 μg/mL, with an observed concentration of 15.04 μg/mL (100.6% of the target value). The samples were analyzed by HPLC using the method parameters described in the table below.

| Item | Value | |
|---|---|---|
| Instrument | Agilent 1260 Infinity w/DAD detector | |
| Column | Waters X Bridge C18, 4.6 × 150 mm, | |
| | 3.6 μm Part #186003034 | |
| Mobile Phase A | H₂O + 0.1% TFA | |
| Mobile Phase B | ACN + 0.1% TFA | |
| Mobile Phase | 45% A, 55% B | |
| Composition (Isocratic) | | |
| Flow | 1.0 | mL/min |
| Injection Volume | 50 | μL |
| Column Temperature | 60° | C. |
| Detector Wavelength | 254 | nm |
| Run Time | 10 | min |

### EXAMPLES

### Identification of Solid Forms of Compound A

Within the pharmaceutical research and development field, the investigation of a suitable solid-state form represents a crucial step. Investigating a solid-state form comprises several decisions, mainly the investigation of an anhydrous, salt or co-crystal form and the investigation of a polymorph of the respective anhydrous, salt or co-crystal. During the drug development process, several properties of research compounds are optimized, typically leading to one or a few candidates that continue into exploratory development programs. Typically, in the assessment and optimization of physical chemical parameters during lead drug development, and one such parameter that is highly focused on is solubility. Beyond the optimization of solubility, further physical chemical parameters, such as (1) melting point, (2) thermal behavior, (3) hygroscopicity, (4) crystal habit, (5) polymorphic behavior or physical stability, (6) impurity profile, and (7) chemical stability of the anhydrous or salt form, must be borne in mind when investigating the solid form. The melting point of a compound, either as a free base, acid or salt form, should meet a certain threshold to allow processing steps such as drying, tableting, or capsuling. The assessment of thermal behavior, which is typically done by thermogravimetry (TGA) and differential scanning calorimetry (DSC), also includes solid-solid phase transitions. These may be either enantiotropic or monotropic and can be related to the conversion of one polymorph to another or one pseudo-polymorph to another pseudo-polymorph—e.g. a lower solvate or hydrate—or a true polymorph. Hygroscopicity plays a key role in the evaluation of solid-state forms, as this property is highly relevant for many process steps such as drying, storage, blending, granulation, to name but a few. Hygroscopicity can be investigated by dynamic vapor sorption (DVS). Basically, this technique yields information on the amount of moisture that is taken up by the compound at a certain relative humidity level. Discussing thermal behavior and hygroscopicity represents the link to another parameter that has to be considered in the selection of a form for pharmaceutical development: for example, a manageable polymorphic behavior is required for an anhydrous or salt form to continue in pharmaceutical development. Therefore, at least a brief assessment of polymorphism is typically carried out in an anhydrous or salt-investigation procedure. In this sense, a manageable polymorphic behavior is not equivalent to the existence of only one or two polymorphic forms, but rather to render a situation where the conversion of polymorphic forms that are not equivalent. Crystal habits can influence anhydrous or salt investigations, and optimization in many cases means moving away a drug in the form of needle-shaped crystals towards e.g. platelets or even cubic crystals exhibiting better flowability. Salt investigation can be a tool to improve impurity profiles of drugs since pharmaceutical salts often exhibit crystal structures that are quite different from the structure of the corresponding free base or acid.

### Solid Form Screen

A solid form screen of HCl and HBr salts of Compound A was conducted. Crystalline salt forms of Compound A were identified, including a crystalline HCl salt form, a crystalline toluene solvate of a crystalline HCl salt form (also referred to herein an HCl salt toluene solvate form of Compound A), a crystalline HBr salt form, a crystalline toluene solvate of a crystalline HBr salt form (also referred to herein an HBr salt toluene solvate form of Compound A). Amorphous salt forms of Compound A were also identified, including an amorphous HCl salt form and an amorphous HBr salt form.

Further, the HBr and HCl crystalline salt forms of Compound A form are the most thermodynamically stable crystalline forms identified in the screening process of the HCl and HBr salts of Compound A. Other crystalline forms identified in this screening process include, HCl and HBr salt toluene solvate forms of Compound A described herein below, and these HCl and HBr salt toluene solvate forms of Compound A convert to the respective HBr and HCl crystalline salt forms of Compound A upon heating.

Specifically, the HCl salt toluene solvate form of Compound A converts to the HCl crystalline salt form of Compound A upon heating under vacuum at about 60° C. for about 2 hours. HBr salt toluene solvate form of Compound A converts to the HCl crystalline salt form of Compound A upon heating under vacuum from about 100° C. to about 125° C. from about 4 to about 24 hours. Further, the DV S data of both crystalline HCl and HBr salts form of Compound A indicate that these two materials are non-hygroscopic.

The higher melting point of HBr crystalline salt form of Compound A (DSC endotherm onset of about 201.1° C.) to the HBr salt toluene solvate form of Compound A (DSC endotherm onset of about 194.5° C.) is a further indicator of its thermodynamic stability HBr crystalline salt form of Compound A. Similarly, the higher melting point of HCl crystalline salt form of Compound A (DSC endotherm onset of about 192.2° C.) to the HCl salt toluene solvate form of Compound A is a further indicator of its thermodynamic stability HCl crystalline salt form of Compound A.

Accordingly, both HBr and HCl crystalline salt forms of Compound A show advantageous properties of the crystalline forms identified in the screening process of the HCl and HBr salts of Compound A.

### Solid Form Screens of HCl and HBr Salt Forms of Compound A

Solid form screens to generate the different solid forms of the HCl and HBr salt forms of Compound A were carried out as described below. The free base of Compound A can be made according to the procedures disclosed in WO 2010/075257 and WO 2016/053890.

### Example 1: Preparation of Crystalline HCL Salt Form 1 of Compound A

Crystalline HCl salt form of Compound A was prepared by adding the free base of Compound A (504.4 mg, 0.86 mmol) and toluene (5 mL) to a scintillation vial and heated to about 62° C. with stirring. Once fully dissolved, 1N aqueous HCl (1.0 mL, 1 mmol) was quickly transferred to the vial. The solution was cooled to room temperature and stirred overnight. The precipitate was filtered, washed with 3×2.5 mL toluene, and vacuum dried at 60° C. overnight to afford a crystalline HCl salt form of Compound A.

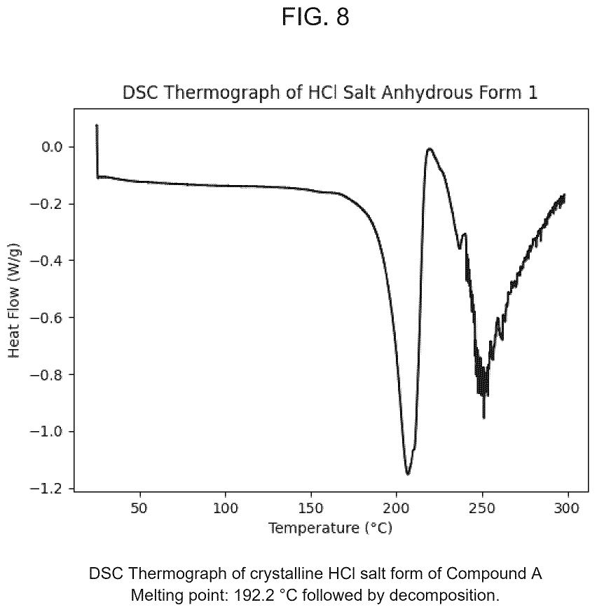

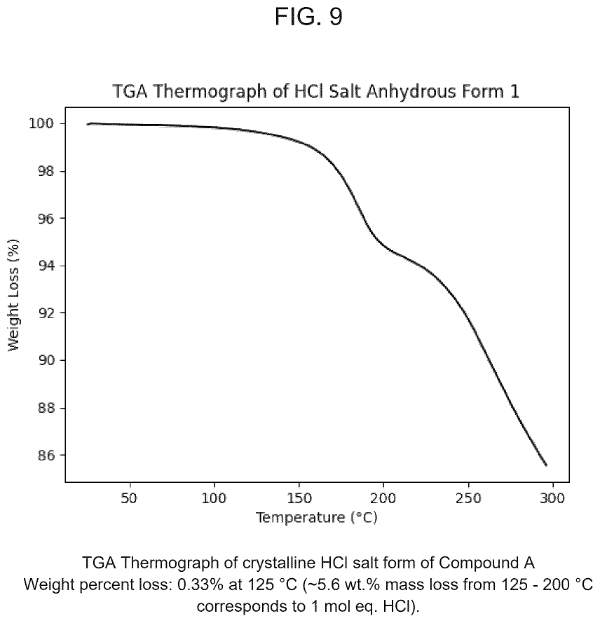

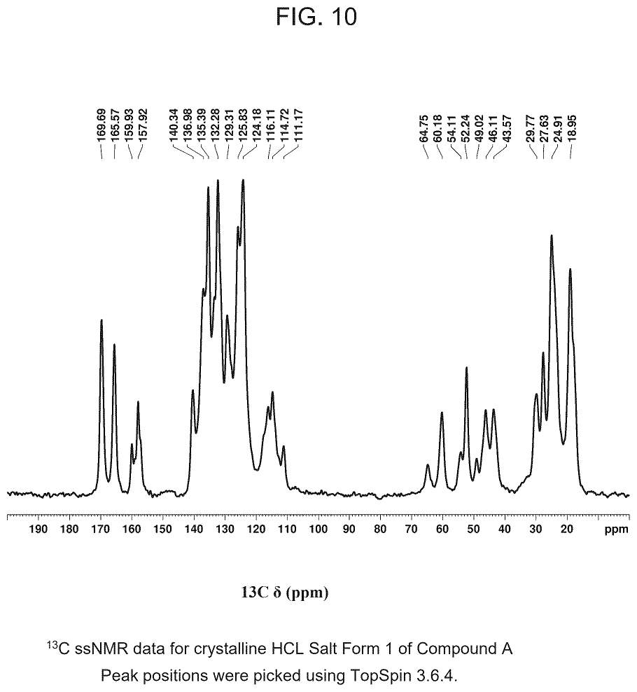

The crystalline HCl salt form of Compound A was characterized by proton NM R, XRPD (FIG. 7), DSC (FIG. 8), TGA (FIG. 9), ¹³C SSNMR (FIG. 10), and DVS (FIG. 26).

The differential scanning calorimetry (DSC) thermogram comprised an endotherm with an onset of about 191° C.±1° C. (FIG. 8).

The thermogravimetric analysis (TGA) thermogram comprised a weight loss of about 0.33%±0.1% when heated from about 25° C. to about 125° C. (FIG. 9).

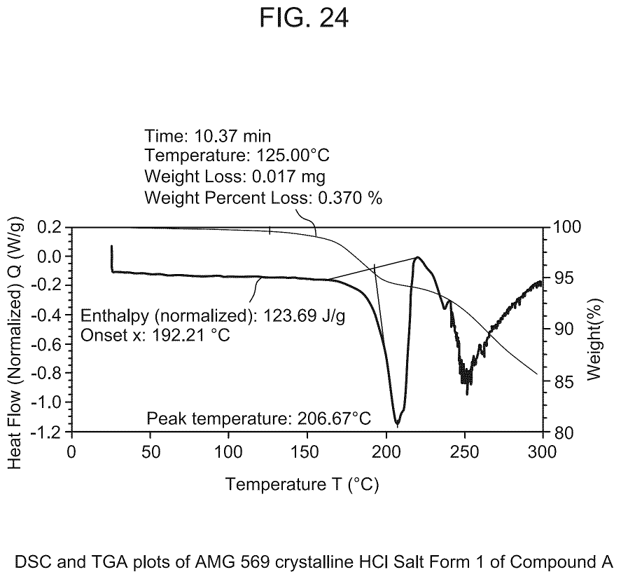

FIG. 24 illustrates the overlay of the DSC and TGA plots of crystalline HCl salt Form 1 of Compound A.

¹H NMR (400 MHz, DMSO-d₆) δ ppm 1.44-1.67 (m, 6H) 1.68-1.75 (m, 2H) 1.82 (m, =12.65, 12.65 Hz, 3H) 1.95 (s, 2H) 1.99-2.24 (m, 1H) 2.35-2.39 (m, 4H) 3.04-3.25 (m, 3H) 3.79 (quin, J=6.70 Hz, 2H) 6.43 (dd, J=12.33, 6.06 Hz, 1H) 7.04-7.22 (m, 4H) 7.31-7.41 (m, 2H) 7.44-7.55 (m, 2H) 7.59-7.69 (m, 1H) 7.89 (dd, J=10.03, 2.09 Hz, 1H) 10.46 (s, 1H)

TABLE 1
XRPD data of Crystalline HCl Salt Form 1 of Compound A
XRPD Peak Table:

| 2θ (°) | Rel. Intensity (%) |
|---|---|
| 5.881 | 27.4 |
| 6.738 | 23.2 |
| 7.346 | 25.2 |
| 10.281 | 12.4 |
| 11.536 | 22.6 |
| 12.894 | 20.2 |
| 13.437 | 46.8 |
| 13.676 | 49.8 |
| 15.384 | 100.0 |
| 16.337 | 82.6 |
| 17.745 | 35.8 |
| 18.722 | 29.4 |
| 19.823 | 15.9 |
| 21.032 | 33.2 |
| 21.850 | 10.6 |
| 22.432 | 35.3 |
| 23.096 | 20.8 |
| 23.843 | 17.1 |
| 25.059 | 19.4 |
| 25.684 | 13.2 |
| 28.259 | 9.1 |

### Example 2: Preparation of Crystalline HBR Salt Form 1 of Compound A

The crystalline HBr salt form of Compound A was prepared by adding free base amorphous form of Compound A (348.6 mg, 0.60 mmol) and acetonitrile (5 mL) to a scintillation vial and heated to about 62° C. with stirring. The solution was cooled to RT and the resulting suspension was stirred for about 30 min. The precipitate was filtered re-slurried in toluene (3 mL) at RT overnight. After a second filtration, the product was washed with 3×2.5 mL toluene and dried at about 125° C. in a fume hood overnight to afford a crystalline HBr salt form of Compound A. Methods of making the free base amorphous form of Compound A is described in U.S. Pat. No. 11,603,356.

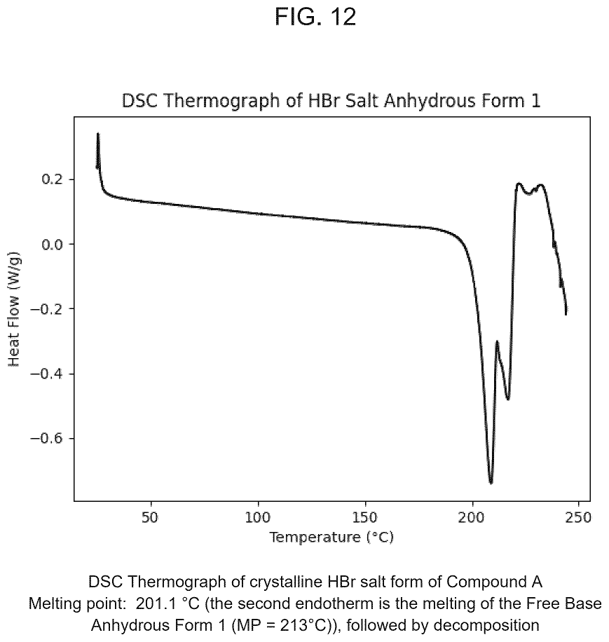

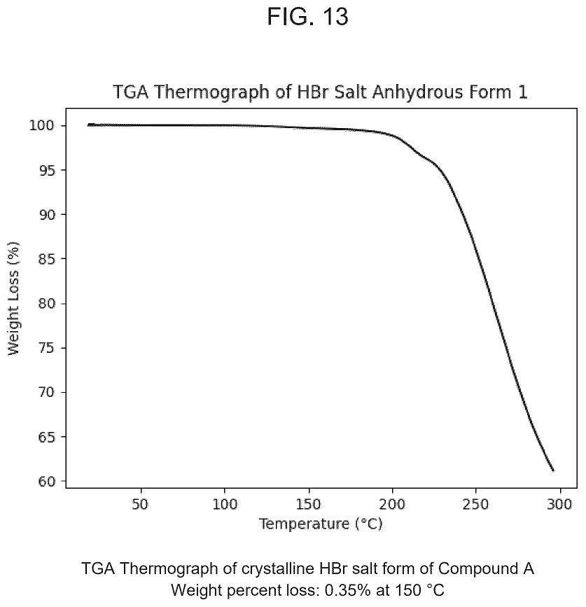

The crystalline HBr salt Form 1 of Compound A was characterized by proton NMR, XRPD (FIG. 11), DSC (FIG. 12), TGA (FIG. 13), ¹³C SSNMR (FIG. 14), and DVS (FIG. 27).

The differential scanning calorimetry (DSC) thermogram comprised an endotherm with an onset of about 201±1° C. (FIG. 12).

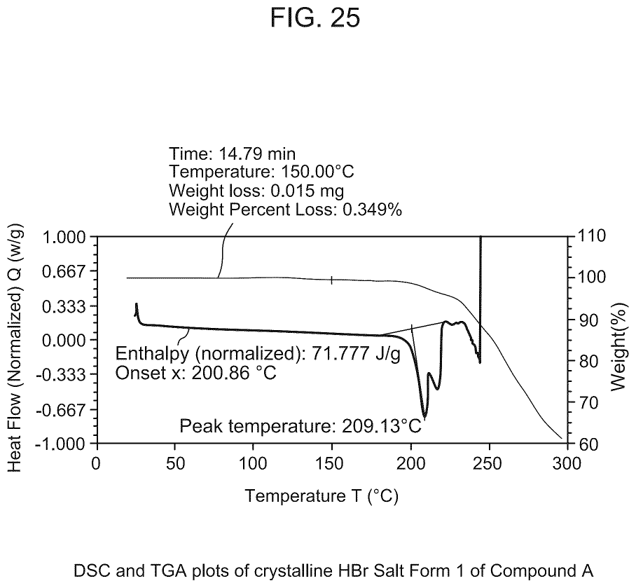

FIG. 25 illustrates the overlay of the DSC and TGA plots of crystalline HBr salt Form 1 of Compound A.

¹H NMR (400 MHz, DMSO-d₆) δ ppm 1.51 (br m, 6H) 1.68 (br m, 2H) 1.76-1.88 (m, 3H) 1.93 (br s, 2H) 1.95-2.18 (m, 1H) 2.34 (br s, 4H) 3.02-3.22 (m, 3H) 3.78 (br s, 2H) 6.36-6.45 (m, 1H) 6.83-7.20 (m, 4H) 7.34 (br d, J=7.11 Hz, 2H) 7.43 (br d, J=12.33 Hz, 2H) 7.62 (br dd, J=19.54, 8.26 Hz, 1H) 7.88 (br d, J=6.06 Hz, 1H) 10.42 (br s, 1H).

TABLE 2
XRPD data of Crystalline HBr Salt Form 1 of Compound A
XRPD Peak Table:

| 2θ (°) | Rel. Intensity (%) |
|---|---|
| 7.514 | 44.9 |
| 10.142 | 19.1 |
| 11.806 | 10.5 |
| 12.487 | 19.5 |
| 12.759 | 26.5 |
| 13.189 | 91.5 |
| 13.620 | 33.2 |
| 15.269 | 100.0 |
| 16.027 | 6.5 |
| 16.417 | 20.1 |
| 17.403 | 13.3 |
| 17.923 | 46.2 |
| 18.783 | 28.5 |
| 19.788 | 11.6 |
| 20.298 | 6.4 |
| 20.894 | 58.8 |
| 21.274 | 26.4 |
| 22.043 | 21.6 |
| 22.858 | 78.8 |
| 23.282 | 12.7 |
| 23.777 | 10.2 |
| 24.672 | 6.5 |
| 25.154 | 32.1 |
| 26.394 | 12.3 |
| 27.802 | 20.0 |
| 28.918 | 8.2 |
| 29.965 | 9.2 |
| 32.765 | 10.7 |
| 34.741 | 9.6 |

### Example 3: Preparation of Crystalline Toluene Solvate of the HCL Salt Form 1 of Compound A

The crystalline toluene solvate of the HCl salt Form 1 of Compound A was prepared by adding 1 equivalent of HCl to the free base amorphous form of Compound A in toluene, and slurrying the reaction mixture at RT for about 24 hours.

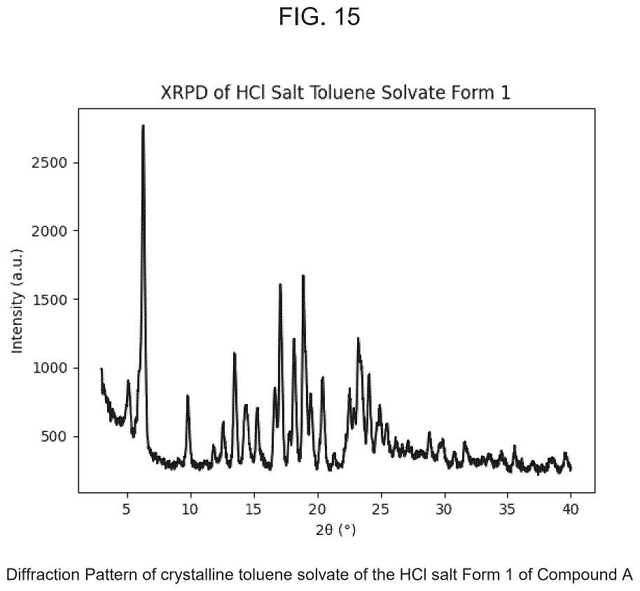

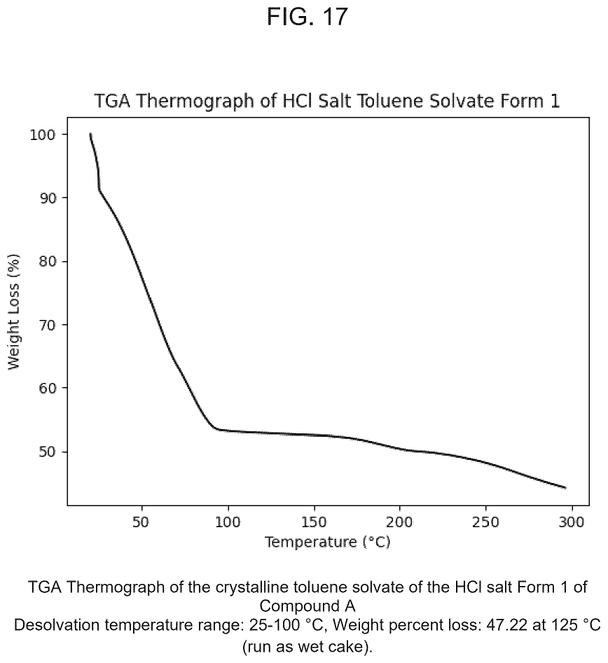

The crystalline toluene solvate of the HCl salt Form 1 of Compound A was characterized by proton NMR, XRPD (FIG. 15) and TGA (FIG. 17).

¹H NMR (400 MHz, DMSO-d₆) δ ppm 1.44-1.67 (m, 6H) 1.72 (br s, 2H) 1.81 (br s, 3H) 1.94 (s, 2H) 1.98-2.22 (m, 1H) 2.31* (s, 14H) 2.35-2.38 (m, 4H) 3.04-3.29 (m, 3H) 3.80 (br dd, J=13.69, 7.00 Hz, 2H) 6.43 (dd, J=12.65, 6.17 Hz, 1H) 7.13-7.20* (m, 16H) 7.22-7.28* (m, 10H) 7.28-7.42 (m, 2H) 7.42-7.57 (m, 2H) 7.58-7.70 (m, 1H) 7.89 (dd, J=10.24, 2.09 Hz, 1H) 10.46 (s, 1H)

TABLE 3
XRPD data of the crystalline toluene solvate
of the HCl salt Form 1 of Compound A
XRPD Peak Table:

| 2θ (°) | Rel. Intensity (%) |
|---|---|
| 5.104 | 15.6 |
| 6.320 | 100.0 |
| 9.809 | 23.3 |
| 11.921 | 6.1 |
| 12.637 | 14.7 |
| 13.533 | 34.7 |
| 14.399 | 17.5 |
| 15.318 | 18.4 |
| 16.680 | 25.1 |
| 17.100 | 59.1 |
| 17.781 | 9.5 |
| 18.207 | 40.2 |
| 18.923 | 63.8 |
| 19.490 | 23.7 |
| 20.454 | 28.1 |
| 21.386 | 5.0 |
| 22.614 | 18.8 |
| 23.356 | 32.6 |
| 24.133 | 25.9 |
| 24.945 | 15.8 |
| 25.486 | 10.3 |
| 26.241 | 3.3 |
| 27.201 | 5.5 |
| 28.871 | 8.8 |
| 29.801 | 6.8 |
| 30.833 | 4.4 |
| 31.716 | 6.6 |
| 35.557 | 8.6 |
| 39.634 | 5.2 |

### Example 4: Preparation of Crystalline Toluene Solvate of the HCL Salt Form 2 of Compound A

The crystalline toluene solvate of the HCl salt Form 2 of Compound A was prepared by heating the crystalline toluene solvate of the HCl salt Form 1 of Compound A at 60° C. for about 24 hours.

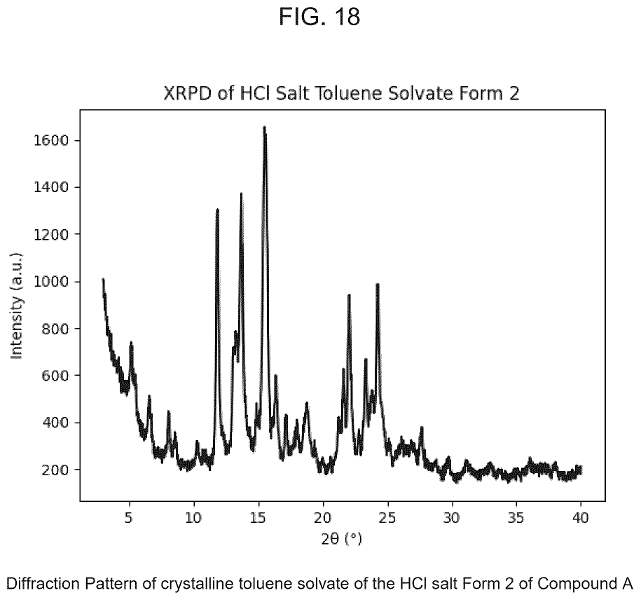

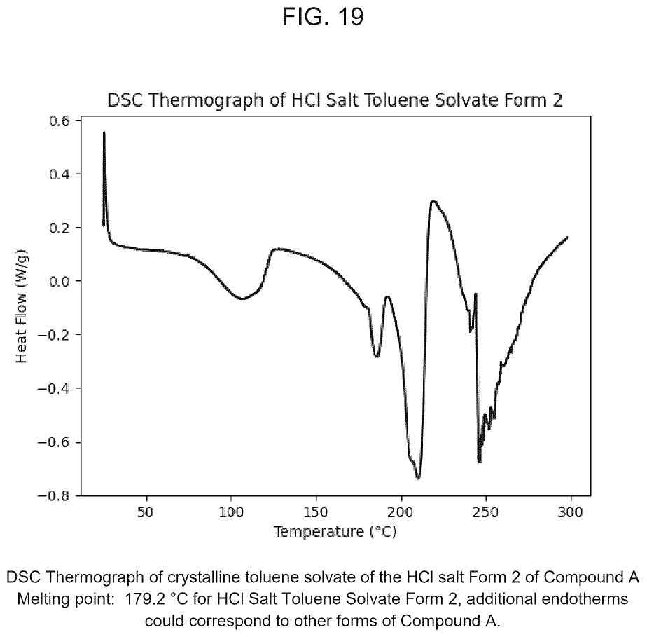

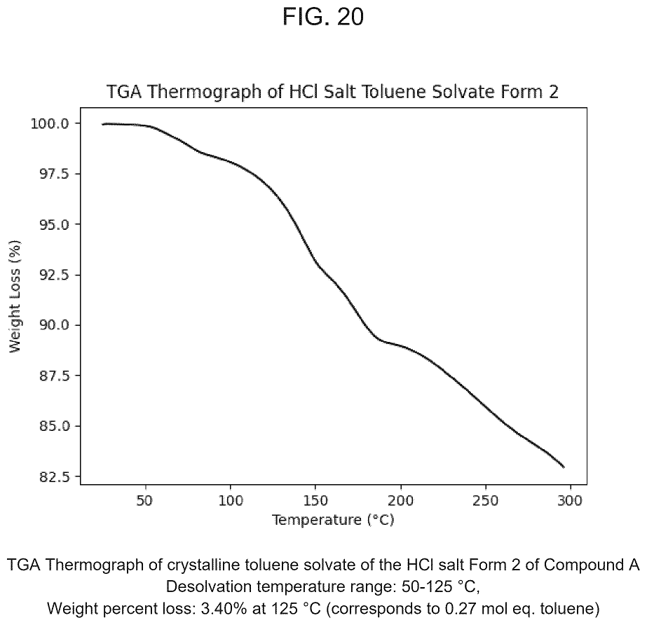

The crystalline toluene solvate of the HCl salt Form 2 of Compound A was characterized by proton NMR, XRPD (FIG. 18), DSC (FIG. 19), and TGA (FIG. 20).

¹H NMR (400 MHz, DMSO-d₆) δ ppm 1.35-1.68 (m, 6H) 1.68-1.75 (m, 2H) 1.75-1.87 (m, 3H) 1.93 (s, 2H) 1.99-2.24 (m, 1H) 2.30* (s, 1H) 2.32-2.40 (m, 4H) 3.04-3.27 (m, 3H) 3.80 (m, j=13.64, 13.64, 6.79 Hz, 2H) 6.43 (dd, j=12.85, 6.17 Hz, 1H) 7.03-7.12 (m, 1H) 7.12-7.21* (m, 2H) 7.21-7.29 (m, 2H) 7.29-7.39* (m, 2H) 7.45-7.58 (m, 2H) 7.57-7.69 (m, 1H) 7.88 (dd, J=10.97, 1.99 Hz, 1H) 10.47 (s, 1H)

TABLE 4
XRPD data of crystalline toluene solvate
of the HCl salt Form 2 of Compound A:
XRPD Peak Table:

| 2θ (°) | Rel. Intensity (%) |
|---|---|
| 5.220 | 18.7 |
| 6.591 | 14.1 |
| 8.093 | 14.2 |
| 8.568 | 9.3 |
| 10.270 | 8.0 |
| 11.864 | 79.7 |
| 13.716 | 84.3 |
| 14.881 | 23.3 |
| 15.556 | 100.0 |
| 16.377 | 28.8 |
| 17.180 | 14.4 |
| 17.999 | 14.5 |
| 18.775 | 19.6 |
| 21.616 | 44.3 |
| 22.059 | 55.9 |
| 22.816 | 14.4 |
| 23.358 | 31.2 |
| 24.265 | 58.3 |
| 27.643 | 11.6 |

### Example 5: Preparation of Crystalline Toluene Solvate of the HBR Salt Form 1 of Compound A

The crystalline toluene solvate of the HBr salt Form of Compound A was prepared by slurring the amorphous HBr salt form of Compound A in toluene at room temperature for 2 days.

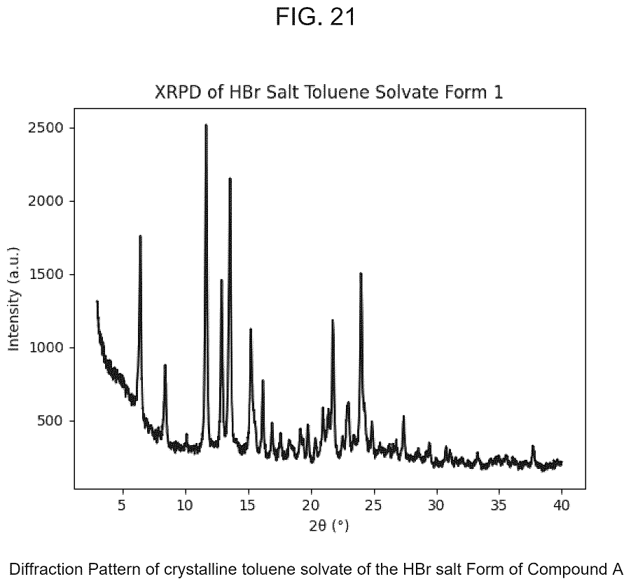

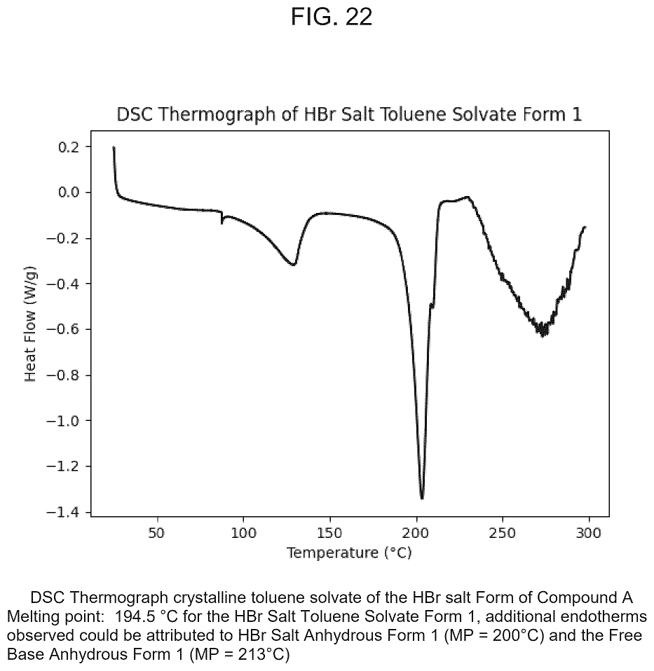

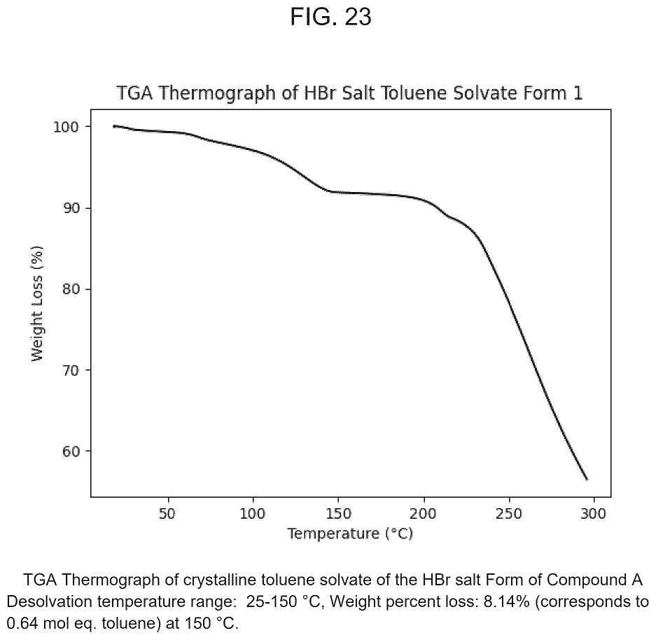

The crystalline toluene solvate of the HBr salt Form of Compound A was characterized by proton NM R, XRPD (FIG. 21), DSC (FIG. 22), and TGA (FIG. 23).

The differential scanning calorimetry (DSC) thermogram comprised an endotherm with an onset of about 195° C.±1° C. (FIG. 22).

The thermogravimetric analysis (TGA) thermogram comprised a weight loss of about 8%±1% when heated from about 25° C. to about 150° C. (FIG. 23).

¹H NMR (400 MHz, DMSO-d₆) δ ppm 1.43-1.60 (m, 6H) 1.63-1.68 (m, 6H) 1.69 (br m, 2H) 1.76-1.86 (m, 3H) 1.93 (s, 2H) 2.01-2.21 (m, 1H) 2.30* (s, 1H) 2.34-2.38 (m, 4H) 3.03-3.28 (m, 3H) 3.78-3.85 (m, 2H) 6.42 (dd, J=11.50, 6.27 Hz, 1H) 7.04-7.21* (m, 5H) 7.21-7.40* (m, 12H) 7.42-7.53 (m, 2H) 7.56-7.68 (m, 1H) 7.89 (dd, J=8.26, 2.19 Hz, 1H) 10.45 (d, J=3.14 Hz, 1H)

TABLE 5
XRPD data of crystalline toluene solvate
of the HBr salt Form of Compound A:
XRPD Peak Table:

| 2θ (°) | Rel. Intensity (%) |
|---|---|
| 6.443 | 52.3 |
| 8.444 | 24.2 |
| 10.137 | 5.1 |
| 11.689 | 100.0 |
| 12.920 | 51.5 |
| 13.588 | 83.0 |
| 15.250 | 37.2 |
| 16.200 | 24.6 |
| 16.942 | 11.0 |
| 17.600 | 8.0 |
| 18.374 | 5.1 |
| 19.184 | 9.4 |
| 19.785 | 11.7 |
| 20.375 | 7.5 |
| 21.000 | 17.5 |
| 21.440 | 15.3 |
| 21.766 | 46.3 |
| 22.603 | 4.1 |
| 23.012 | 16.4 |
| 24.029 | 60.9 |
| 24.881 | 11.2 |
| 27.421 | 13.6 |
| 29.445 | 7.9 |
| 30.791 | 5.5 |
| 31.099 | 6.1 |
| 33.314 | 4.4 |
| 37.711 | 7.1 |

### Example 6: Preparation of the Amorphous HCL Salt Form 2 of COMPOUND A

The amorphous HCl salt form of Compound A was prepared by adding 1 equivalent of HCl to the amorphous free base form of Compound A in ACN at room temperature for about 24 hours.

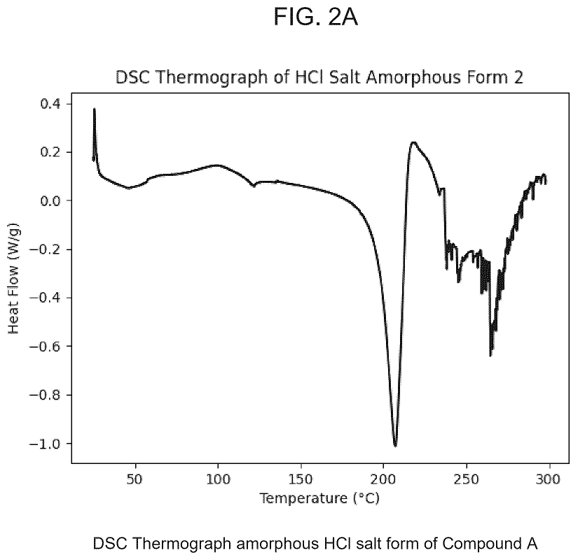

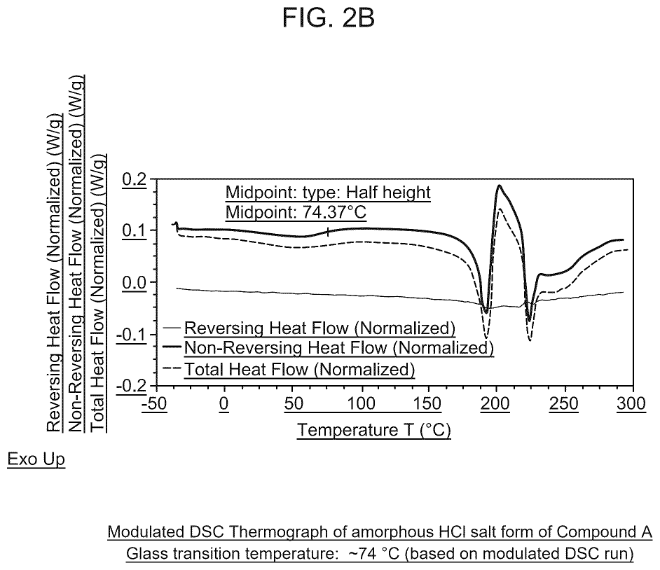

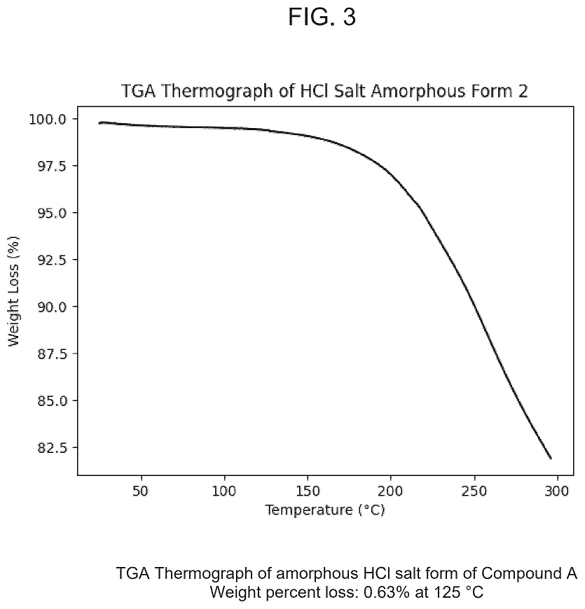

The amorphous HCl salt form of Compound A was characterized by proton NMR, X-ray powder diffraction (XRPD) data (FIG. 1), DSC (FIGS. 2A and 2B), and TGA (FIG. 3).

A modulated differential scanning calorimetry thermogram comprised a glass transition temperature of about 74±1° C. (FIG. 2B).

A thermogravimetric analysis thermogram comprised a weight loss of about 0.6%±0.1% when heated from about 25° C. to about 125° C. (FIG. 3).

¹H NMR (500 MHz, DMSO-d₆) δ ppm 1.40-1.67 (m, 6H) 1.67-1.76 (m, 2H) 1.77-1.86 (m, 3H) 1.94 (s, 2H) 1.99-2.24 (m, 1H) 2.32-2.40 (m, 4H) 3.03-3.21 (m, 3H) 3.79 (dt, J=13.85, 6.98 Hz, 2H) 6.43 (dd, J=12.65, 6.17 Hz, 1H) 7.04-7.30 (m, 4H) 7.31-7.39 (m, 2H) 7.43-7.55 (m, 2H) 7.58-7.69 (m, 1H) 7.89 (dd, J=10.45, 2.09 Hz, 1H) 10.46 (s, 1H)

### Example 7: Preparation of the Amorphous HBR Salt Form of Compound A

The amorphous HBr salt form of Compound A was prepared by adding 1 equivalent of HBr to the free base amorphous form of Compound A in ACN and slurring at room temperature for 24 hours.

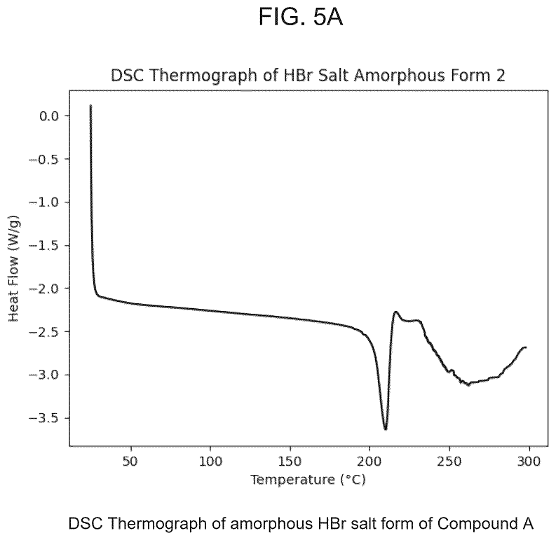

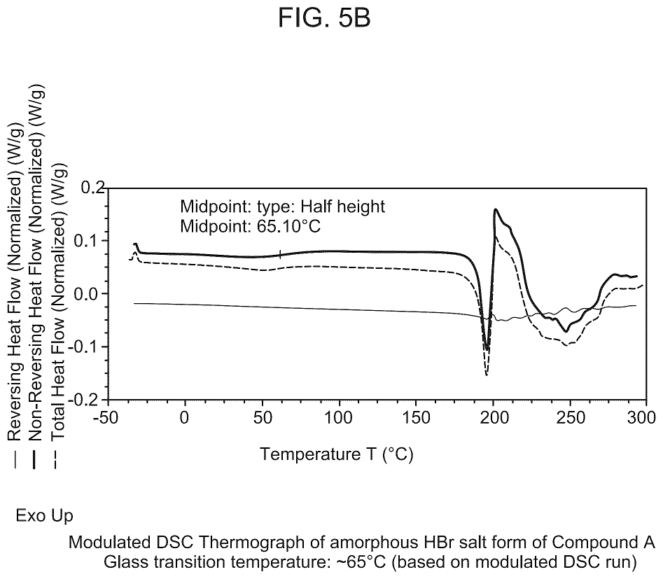

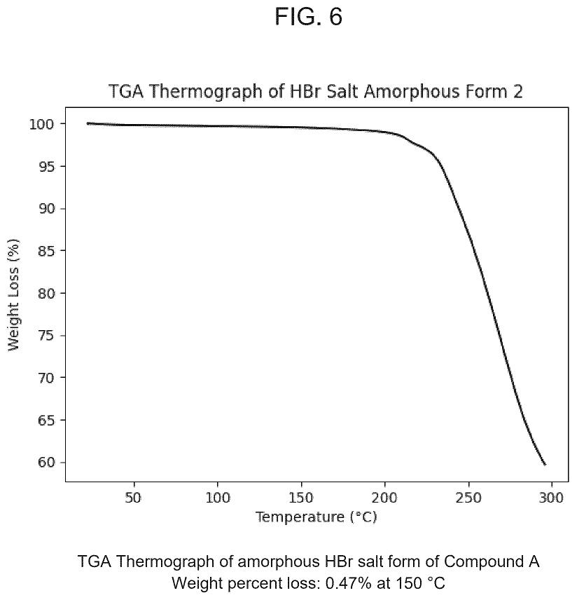

The amorphous HBr salt form of Compound A was characterized by proton NMR, X-ray powder diffraction (XRPD) data (FIG. 4), DSC (FIGS. 5A and 5B), and TGA (FIG. 6).

A modulated differential scanning calorimetry thermogram comprised a glass transition temperature of about 65±1° C. (FIG. 5B).

A thermogravimetric analysis thermogram comprised a weight loss of about 0.5%±0.1% when heated from about 25° C. to about 505° C. (FIG. 6).

¹H NMR (400 MHz, DMSO-d₆) δ ppm 1.53 (br m, 6H) 1.63-1.75 (m, 2H) 1.78-1.87 (m, 3H) 1.94 (s, 2H) 2.08* (s, 2H) 2.15 (td, J=13.22, 4.08 Hz, 1H) 2.34-2.39 (m, 4H) 3.04-3.25 (m, 3H) 3.74-3.86 (m, 2H) 6.42 (dd, J=11.50, 6.06 Hz, 1H) 6.94-7.20 (m, 4H) 7.30-7.39 (m, 2H) 7.45 (m, j=18.29, 6.38 Hz, 2H) 7.58-7.68 (m, 1H) 7.89 (dd, j=7.94, 2.09 Hz, 1H) 10.44 (d, j=3.55 Hz, 1H)

### Example 8: Preparation of Crystalline HCL Salt Form 3 of Compound A

Compound A was charged to a vial and then ethanol was charged to the vial until dissolution of Compound A occurred. 2 equivalences of 2N aqueous HCl was charged to the vial. The vial was placed on a heated stir plate. The vial was agitated with magnetic stirring and heated slowly to 70 C° over about an hour. The vial was cooled to 35 C° and held overnight. The above heating cycle procedure (heated slowly to 70 C° over about an hour and cooled to 35 C° and held overnight) was performed again the following day. The resultant slurry was filtered.

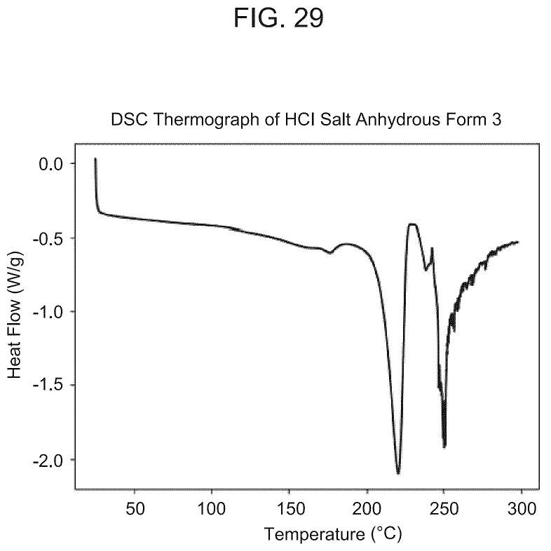

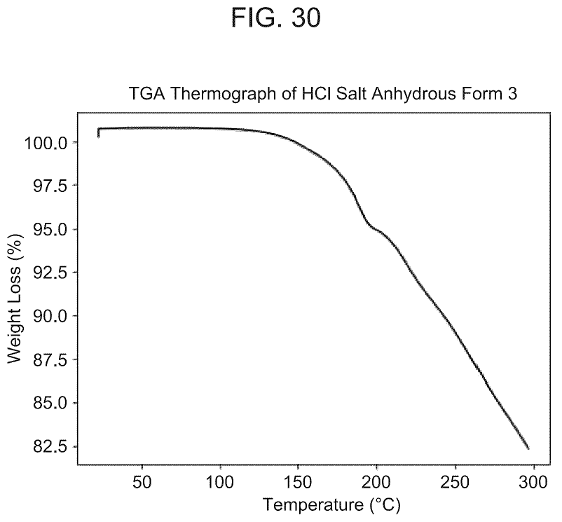

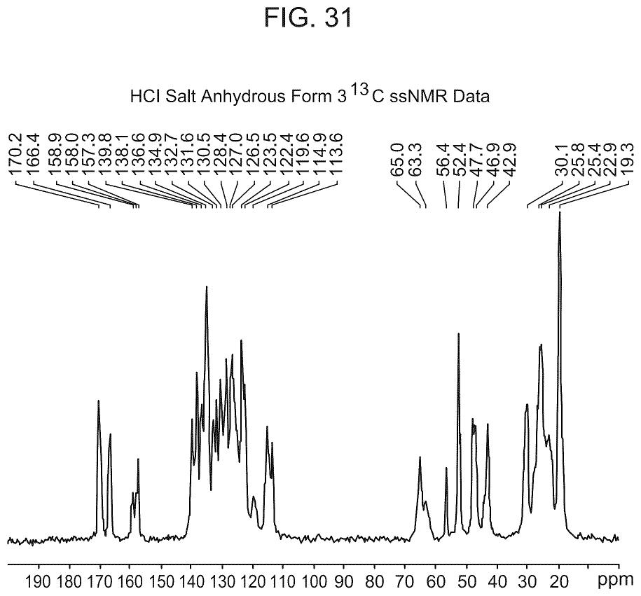

The crystalline HCl salt Form 3 of Compound A was characterized by ¹³C ssNMR (FIG. 31), XRPD (FIG. 28), DSC (FIG. 29), and TGA (FIG. 30).

The differential scanning calorimetry (DSC) thermogram comprised an endotherm with an onset of about 192° C.±1° C. (FIG. 29).

The thermogravimetric analysis (TGA) thermogram comprised a weight loss of about of about 0.2 wt. %±0.1% when heated from about 25° C. to about 150° C. (FIG. 30).

TABLE 6
XRPD data of crystalline HCl salt Form 3 of Compound A:
XRPD Peak Table:

| 2θ° | Rel. Intensity (%) |
|---|---|
| 3.9 | 100 |
| 5.2 | 28 |
| 7.7 | 22 |
| 10.5 | 40 |
| 11.0 | 21 |
| 11.7 | 18 |
| 12.3 | 39 |
| 12.6 | 64 |
| 13.9 | 23 |
| 14.6 | 20 |
| 15.0 | 24 |
| 15.6 | 70 |
| 17.1 | 15 |
| 18.1 | 32 |
| 18.6 | 83 |
| 19.6 | 27 |
| 20.2 | 19 |
| 21.5 | 32 |
| 21.9 | 26 |
| 22.9 | 19 |
| 24.5 | 15 |
| 25.2 | 34 |
| 27.1 | 15 |
| 27.9 | 13 |
| 29.4 | 19 |
| 35.7 | 14 |

TABLE 7
¹³C ssNMR data of crystalline HCl salt Form 3 of Compound A:
¹³C ssNMR data Peak Table:

| Peak | ν(F1) [ppm] | Intensity [abs] |
|---|---|---|
| 1 | 170.2 | 9169391 |
| 2 | 166.4 | 6982544 |
| 3 | 158.9 | 3088180 |
| 4 | 158.0 | 3096289 |
| 5 | 157.3 | 5300046 |
| 6 | 139.8 | 7968344 |
| 7 | 138.1 | 11052992 |
| 8 | 136.6 | 8925288 |
| 9 | 134.9 | 16755488 |
| 10 | 132.7 | 7965057 |
| 11 | 131.6 | 9263093 |
| 12 | 130.5 | 10596855 |
| 13 | 128.4 | 11949130 |
| 14 | 127.0 | 10757930 |
| 15 | 126.5 | 12226728 |
| 16 | 123.5 | 13148431 |
| 17 | 122.4 | 10274459 |
| 18 | 119.6 | 2828750 |
| 19 | 114.9 | 7496381 |
| 20 | 113.6 | 6415068 |
| 21 | 65.0 | 5451826 |
| 22 | 63.3 | 2483351 |
| 23 | 56.4 | 4704864 |
| 24 | 52.4 | 13631227 |
| 25 | 47.7 | 8030867 |
| 26 | 46.9 | 7589570 |
| 27 | 42.9 | 7637283 |
| 28 | 30.1 | 8939145 |
| 29 | 25.8 | 12797249 |
| 30 | 25.4 | 12878234 |
| 31 | 22.9 | 6879297 |
| 32 | 19.3 | 23250691 |

### Example 9: Preparation of the HCL Salt THF/H₂0 Solvate Form 1 of Compound A

582 mg of compound A was dissolved in 2 mL TH F at room temperature. To this solution around 6 mL (3 eq) of 1N aq HCl was added. Around 6 mL of water was added and stirred at room temperature. After 6 days, a crystalline slurry was isolated.

The HCl salt THF/H₂O solvate form of Compound A was characterized by ¹³C ssNMR, X-ray powder diffraction (XRPD) data (FIG. 4), DSC (FIGS. 5A and 5B), and TGA (FIG. 6).

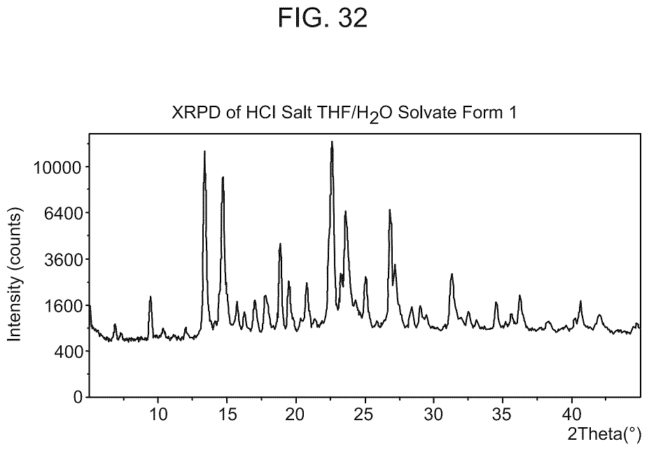

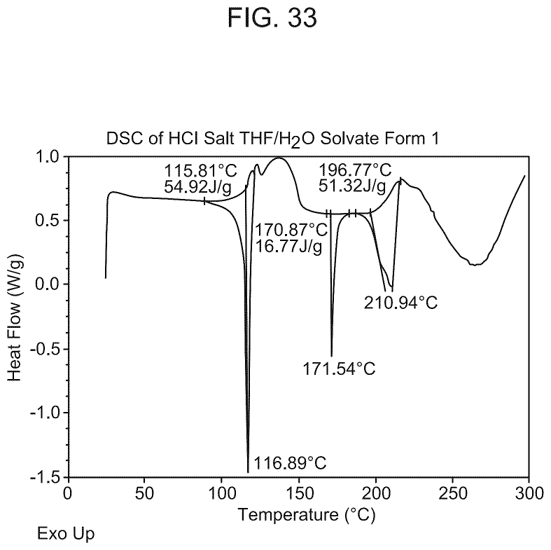

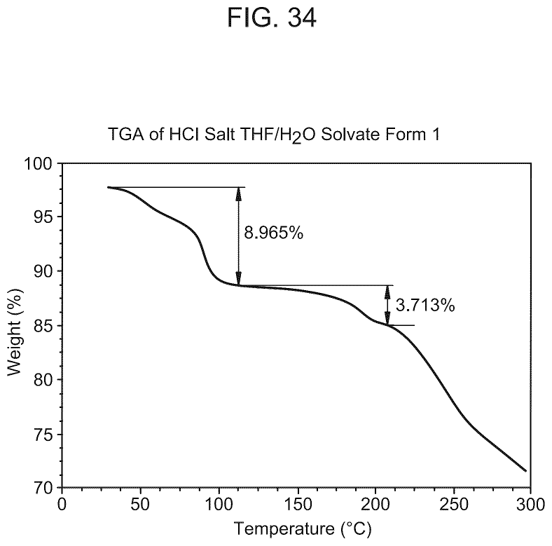

The crystalline HCl salt THF/H₂O Form of Compound A was characterized by, XRPD (FIG. 32), DSC (FIG. 33), and TGA (FIG. 34).

The differential scanning calorimetry (DSC) thermogram comprised an endotherm with an onset of about 117° C.±1° C. (FIG. 33).

The thermogravimetric analysis (TGA) thermogram comprised a weight loss of about of about 9%±1% when heated from about 25° C. to about 100° C. (FIG. 34).

TABLE 8
XRPD data of crystalline toluene solvate
of the HCl salt THF/H₂O Form of Compound A:
XRPD Peak Table:

| 2θ° | Rel. Intensity (%) |
|---|---|
| 9.4 | 16 |
| 13.4 | 93 |
| 14.7 | 74 |
| 15.7 | 14 |
| 17 | 14 |
| 17.8 | 16 |
| 18.9 | 36 |
| 19.5 | 21 |
| 20.8 | 20 |
| 22.6 | 100 |
| 23.6 | 53 |
| 25 | 22 |
| 26.8 | 54 |
| 27.2 | 27 |
| 28.3 | 13 |
| 29 | 13 |
| 31.3 | 23 |
| 34.5 | 14 |
| 36.2 | 16 |
| 40.6 | 15 |

### Example 10: Solubility Studies

Solubility: Samples were prepared by adding ˜10 mg of the solid to a small centrifuge tube followed by the addition of 1.75 mL freshly prepared FaSSIF solution. The pH of each suspension was measured prior to shaking the samples in an Eppendorf ThermoMixer F1.5 at 37° C. and 1500 rpm for 2 hr. The samples were centrifuged, and the pH of each suspension was measured again at the T=2 hr timepoint. The samples were filtered through Millex 0.20 μm PTFE syringe filters and the filtrates were diluted by a factor of 2 with acetonitrile. Solubility samples were prepared in duplicate. Calibration standards were prepared at API concentrations of 1, 2.5, 7.5, 15, 25, and 50 μg/mL using 1:1 H2O/ACN as a diluent (linear regression R2=0.999965). A check standard was prepared at a concentration of 14.94 μg/mL, with an observed concentration of 15.04 μg/mL (100.6% of the target value). The samples were analyzed by HPLC using the method parameters described in the table below.

| Item | Value | |
|---|---|---|
| Instrument | Agilent 1260 Infinity w/DAD detector | |
| Column | Waters X Bridge C18, 4.6 × 150 mm, | |
| | 3.6 μm Part #186003034 | |
| Mobile Phase A | H₂O + 0.1% TFA | |
| Mobile Phase B | ACN +0.1% TFA | |
| Mobile Phase | 45% A, 55% B | |
| Composition (Isocratic) | | |
| Flow | 1.0 | mL/min |
| Injection Volume | 50 | μL |
| Column Temperature | 60° | C. |
| Detector Wavelength | 254 | nm |
| Run Time | 10 | min |

The solubility of the following crystalline and amorphous forms of Compound A were compared:

| Initial Form of | | | Temperature | Solubility | Equilibrium |
|---|---|---|---|---|---|
| Compound A | Solvent | pH | (° C.) | (μg/ml) | Form |
| Free Base | FaSSIF | 6.524 | 37 | 1.71 | Free Base |
| Anhydrous | | | | | Anhydrous |
| Crystalline | | | | | Crystalline |
| Form | | | | | Form |
| Free Base | FaSSIF | 6.513 | 37 | 19.44 | Free Base |
| Amorphous | | | | | Amorphous |
| Form | | | | | Form |
| Crystalline | FaSSIF | 6.433 | 37 | 18.50* | Crystalline |
| HCl salt form | | | | | HCl salt |
| | | | | | form |
| Crystalline | FaSSIF | 6.421 | 37 | 18.84* | Crystalline |
| HBr salt form | | | | | HBr salt |
| | | | | | form |

*Note:
Solubility is not corrected for counterion content

Besylate, Tosylate Napsylate salt forms of Compound A were prepared as described in WO 2021/092295. The solubility of the crystalline HCl salt Form 1 of Compound A described in this disclosure and crystalline HBr Salt Form 1 of Compound A described in this disclosure were compared to the Besylate, Tosylate, and Napsylate crystalline salt forms of Compound:

Salt Solubility Data for Compound A

| Initial | | | Temperature | Solubility * | Equilibrium |
|---|---|---|---|---|---|
| Form | Media | pH | (° C.) | (μg/mL) | Form |
| HCl Salt | FaSSIF | 6.450 | 37 | 17.45 | HCl Salt |
| Anhydrous | | | | | Anhydrous |
| Form 1 | | | | | Form 1 |
| HBr Salt | FaSSIF | 6.445 | 37 | 16.26 | HBr Salt |
| Anhydrous | | | | | Anhydrous |
| Form 1 | | | | | Form 1 |
| Besylate | FaSSIF | 6.479 | 37 | 15.12 | Besylate |
| Anhydrous | | | | | Anhydrous |
| Form | | | | | Form |
| Tosylate | FaSSIF | 6.472 | 37 | 14.94 | Tosylate |
| Anhydrous | | | | | Anhydrous |
| Form | | | | | Form |
| Napsylate | FaSSIF | 6.469 | 37 | 15.57 | Napsylate |
| Anhydrous | | | | | Anhydrous |
| Form | | | | | Form |

* Solubility of salts is not corrected for counterion content

In the table above, the HCl and HBr salt forms of Compound A were significantly more soluble than the Besylate, Tosylate and Napsylate forms.

While the invention has been described and illustrated with reference to certain particular embodiments thereof, those skilled in the art will appreciate that various adaptations, changes, modifications, substitutions, deletions, or additions of procedures and protocols may be made without departing from the spirit and scope of the disclosure. It is intended, therefore, that the invention be defined by the scope of the claims that follow and that such claims be interpreted as broadly as is reasonable.

### Example 11: Dissolution Studies

Dissolution profiles were generated comparing the free base anhydrous crystalline form of Compound A, the amorphous free base form of Compound A, the crystalline HCl salt form of Compound A, and the crystalline HBr salt form of Compound A. The dissolution profiles were obtained by employing a PION MicroDiss profiler equipped with Rainbow R2D 8-channel in-situ fiber optic UV-vis spectrometer, heating blocks and magnetic stirrers. Dissolution media was equilibrated at 37° C. using Thermo Neslab RTE 7 recirculating chiller prior to addition of samples. Calibration standards were prepared by serial dilution from a methanol stock solution at 12 levels spanning concentrations 0-38 μg/mL. Samples were analyzed simultaneously in duplicate using the parameters described in the table below.

| Item | Value | |
|---|---|---|
| Instrument | Pion Rainbow R2D | |
| Temperature | ~37° | C. |
| Stirring rate | 250 | rpm |
| Wavelength range | 254-256 | nm |
| Media | FaSSIF + 0.2% Tween 80 | |
| Run time | 18 | hr |
| Probe path length | 5 | mm |
| Sample mass | ~2.5 | mg |
| Volume | 10.0 | mL |

| | |
|---|---|
| Baseline correction algorithm | Point at 300 nm |

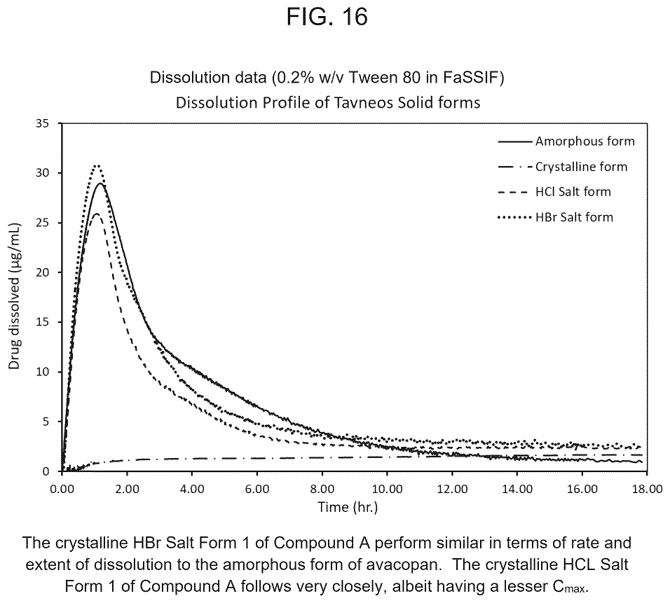

The resulting dissolution profiles of these forms of Compound A are shown in FIG. 16. From these data, it was observed that the crystalline HBr salt form of Compound A unexpectedly performed similar in terms of rate and extent of dissolution to the amorphous form of avacopan. The crystalline HCl salt form of Compound A follows very closely, albeit having a lesser Cmax.

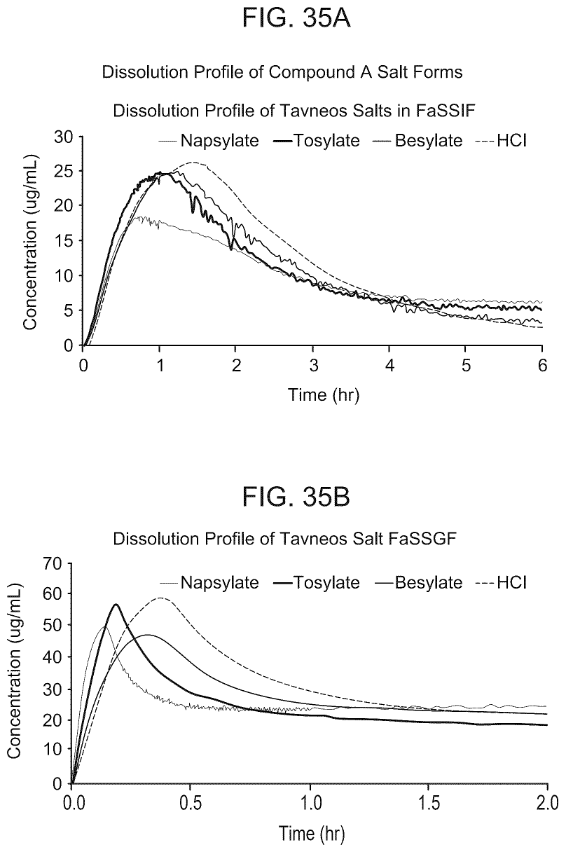

FIGS. 35(a) and 35(b) show dissolution profiles of crystalline HCl Form 1 of Compound A of this disclosure compared to Napsylate, Tosylate, and Besylate salt forms of Compound A. Besylate, Tosylate Napsylate salt forms of Compound A were prepared as described in WO 2021/092295. The concentrations of the salts used are roughly double in FIG. 35(b) than in FIG. 35(a). Crystalline HCl salt Form 1 of Compound A had the highest Cmax of all salts in both FaSSIF+0.2% tween 80 (pH 6.5) and FaSSGF+0.2% tween 80 (pH 1.2). The Tmax of the crystalline HCl salt Form 1 of Compound A was also higher than the Napsylate, Tosylate, and Besylate salt forms of Compound A.

While the invention has been described and illustrated with reference to certain particular embodiments thereof, those skilled in the art will appreciate that various adaptations, changes, modifications, substitutions, deletions, or additions of procedures and protocols may be made without departing from the spirit and scope of the disclosure. It is intended, therefore, that the invention be defined by the scope of the claims that follow and that such claims be interpreted as broadly as is reasonable.

What is claimed is:

1. A crystalline salt form of (2R,3S)-2-(4-(cyclopentylamino)phenyl)-1-(2-fluoro-6-methylbenzoyl)-N-(4-methyl-3-(trifluoromethyl)phenyl)piperidine-3-carboxamide (Compound A), wherein the crystalline salt form of Compound A is a crystalline HCl salt form or a crystalline HBr salt form.

2. The crystalline HCl salt form of claim 1, wherein the crystalline HCl salt form is anhydrous.

3. The crystalline HCl salt form of claim 1, wherein the crystalline HCl salt form is characterized by a powder X-ray diffraction pattern comprising at least one peak selected from 5.9, 7.3, 13.4, 13.7, 15.4, 16.3, 17.7, 18.7, 21.0, and 22.4±0.2 degrees 2 theta as measured by X-ray powder diffraction using an X-ray wavelength of 1.54 Å.

4. The crystalline HCl salt form of claim 1, wherein the crystalline HCl salt form is characterized by a powder X-ray diffraction pattern comprising at least two peaks selected from 5.9, 7.3, 13.4, 13.7, 15.4, 16.3, 17.7, 18.7, 21.0, and 22.4±0.2 degrees 2 theta as measured by X-ray powder diffraction using an X-ray wavelength of 1.54 Å.

5. The crystalline HCl salt form of claim 1, wherein the crystalline HCl salt form is characterized by a powder X-ray diffraction pattern comprising at least three peaks selected from 5.9, 7.3, 13.4, 13.7, 15.4, 16.3, 17.7, 18.7, 21.0, and 22.4±0.2 degrees 2 theta as measured by X-ray powder diffraction using an X-ray wavelength of 1.54 Å.

6. The crystalline HCl salt form of claim 1, wherein the crystalline HCl salt form is characterized by a powder X-ray diffraction pattern comprising peaks at 5.9, 7.3, 13.4, 13.7, 15.4, 16.3, 17.7, 18.7, 21.0, and 22.4±0.2 degrees 2 theta as measured by X-ray powder diffraction using an X-ray wavelength of 1.54 Å.

7. The crystalline HCl salt form of claim 1, wherein the crystalline HCl salt form is characterized by a powder X-ray diffraction pattern comprising at least three peaks selected from 5.9, 6.7, 7.3, 10.3, 11.5, 12.9, 13.4, 13.7, 15.4, 16.3, 17.7, 18.7, 19.8, 21.0, 21.9, 22.4, 23.1, 23.8, 25.7, and 28.3±0.2 degrees 2 theta as measured by X-ray powder diffraction using an X-ray wavelength of 1.54 Å.

8. The crystalline HCl salt form of claim 1, wherein the crystalline HCl salt form is characterized by a powder X-ray diffraction pattern comprising at least five peaks selected from 5.9, 6.7, 7.3, 10.3, 11.5, 12.9, 13.4, 13.7, 15.4, 16.3, 17.7, 18.7, 19.8, 21.0, 21.9, 22.4, 23.1, 23.8, 25.7, and 28.3±0.2 degrees 2 theta as measured by X-ray powder diffraction using an X-ray wavelength of 1.54 Å.

9. The crystalline HCl salt form of claim 1, wherein the crystalline HCl salt form is characterized by a powder X-ray diffraction pattern comprising at least seven peaks selected from 5.9, 6.7, 7.3, 10.3, 11.5, 12.9, 13.4, 13.7, 15.4, 16.3, 17.7, 18.7, 19.8, 21.0, 21.9, 22.4, 23.1, 23.8, 25.7, and 28.3±0.2 degrees 2 theta as measured by X-ray powder diffraction using an X-ray wavelength of 1.54 Å.

10. The crystalline HCl salt form of claim 1, wherein the crystalline HCl salt form is characterized by a powder X-ray diffraction pattern comprising peaks at 5.9, 6.7, 7.3, 10.3, 11.5, 12.9, 13.4, 13.7, 15.4, 16.3, 17.7, 18.7, 19.8, 21.0, 21.9, 22.4, 23.1, 23.8, 25.7, and 28.3±0.2 degrees 2 theta as measured by X-ray powder diffraction using an X-ray wavelength of 1.54 Å.

11. The crystalline HCl salt form of claim 1, wherein the crystalline HCl salt form is characterized by the powder X-ray diffraction pattern substantially as shown in FIG. 7 as measured by X-ray powder diffraction using an X-ray wavelength of 1.54 Å.

12. The crystalline HCl salt form of claim 1, wherein the crystalline HCl salt form of is characterized by a differential scanning calorimetry thermogram comprising an endotherm with an onset of about 192±1° C.

13. The crystalline HCl salt form of claim 1, wherein the crystalline HCl salt is characterized by a thermogravimetric analysis thermogram comprising a weight loss of about 0.33%±0.1% when heated from about 25° C. to about 125° C.

14. The crystalline HCl salt form of claim 13, wherein the thermogravimetric analysis thermogram further comprising a weight loss of about 5.6%±0.5% from about 125° C. to about 200° C.

15. The crystalline HCl salt of claim 1, wherein the crystalline HCl salt form is characterized by ¹³C solid state NMR comprising at least three peaks selected from peaks at approximately 170, 166, 160, 158, 140, 137, 135, 132, 129, 126, 124, 116, 115, 111, 65, 60, 54, 52, 49, 46, 44, 30, 28, 25, and 19 ppm.

16. The crystalline HCl salt of claim 1, wherein the crystalline HCl salt form is characterized by ¹³C solid state NMR comprising at least five peaks selected from peaks at approximately 170, 166, 160, 158, 140, 137, 135, 132, 129, 126, 124, 116, 115, 111, 65, 60, 54, 52, 49, 46, 44, 30, 28, 25, and 19 ppm.

17. The crystalline HCl salt of claim 1, wherein the crystalline HCl salt form is characterized by ¹³C solid state NMR comprising at least seven peaks selected from peaks at approximately 170, 166, 160, 158, 140, 137, 135, 132, 129, 126, 124, 116, 115, 111, 65, 60, 54, 52, 49, 46, 44, 30, 28, 25, and 19 ppm.

18. The crystalline HCl salt of claim 1, wherein the crystalline HCl salt form is characterized by ¹³C solid state NMR comprising peaks at approximately 170, 166, 160, 158, 140, 137, 135, 132, 129, 126, 124, 116, 115, 111, 65, 60, 54, 52, 49, 46, 44, 30, 28, 25, and 19 ppm.

19. The crystalline HCl salt form of claim 1, wherein the crystalline HCl salt form is substantially free of other crystalline or amorphous forms.

20. The crystalline HCl salt form of claim 19, wherein the amount of other crystalline or amorphous forms is 5% (w/w) or less.

21. The crystalline HBr salt form of claim 1, wherein the crystalline HBr salt form is anhydrous.

22. The crystalline HBr salt form of claim 1, wherein the crystalline HBr salt form is characterized by a powder X-ray diffraction pattern comprising at least one peak selected from 7.5, 12.8, 13.2, 13.6, 15.3, 17.9, 18.8, 20.9, 21.3, 22.9, and 25.2±0.2 degrees 2 theta as measured by X-ray powder diffraction using an X-ray wavelength of 1.54 Å.

23. The crystalline HBr salt form of claim 1, wherein the crystalline HBr salt form is characterized by a powder X-ray diffraction pattern comprising at least two peaks selected from 7.5, 12.8, 13.2, 13.6, 15.3, 17.9, 18.8, 20.9, 21.3, 22.9, and 25.2±0.2 degrees 2 theta as measured by X-ray powder diffraction using an X-ray wavelength of 1.54 Å.

24. The crystalline HBr salt form of claim 1, wherein the crystalline HBr salt form is characterized by a powder X-ray diffraction pattern comprising at least three peaks selected from 7.5, 12.8, 13.2, 13.6, 15.3, 17.9, 18.8, 20.9, 21.3, 22.9, and 25.2±0.2 degrees 2 theta as measured by X-ray powder diffraction using an X-ray wavelength of 1.54 Å.

25. The crystalline HBr salt form of claim 1, wherein the crystalline HBr salt form is characterized by a powder X-ray diffraction pattern comprising peaks at 7.5, 12.8, 13.2, 13.6, 15.3, 17.9, 18.8, 20.9, 21.3, 22.9, and 25.2±0.2 degrees 2 theta as measured by X-ray powder diffraction using an X-ray wavelength of 1.54 Å.

26. The crystalline HBr salt form of claim 1, wherein the crystalline HBr salt form is characterized by a powder X-ray diffraction pattern comprising at least three peaks selected from 7.5, 10.1, 11.8, 12.5, 12.8, 13.2, 13.6, 15.3, 16.0, 16.4, 17.4, 17.9, 18.8, 19.8, 20.3, 20.9, 21.3, 22.0, 22.9, 23.3, 23.8, 24.7, 25.2, 26.4, 27.8, 28.9, 30.0, 32.8, and 34.7±0.2 degrees 2 theta as measured by X-ray powder diffraction using an X-ray wavelength of 1.54 Å.

27. The crystalline HBr salt form of claim 1, wherein the crystalline HBr salt form is characterized by a powder X-ray diffraction pattern comprising at least five peaks selected from 7.5, 10.1, 11.8, 12.5, 12.8, 13.2, 13.6, 15.3, 16.0, 16.4, 17.4, 17.9, 18.8, 19.8, 20.3, 20.9, 21.3, 22.0, 22.9, 23.3, 23.8, 24.7, 25.2, 26.4, 27.8, 28.9, 30.0, 32.8, and 34.7±0.2 degrees 2 theta as measured by X-ray powder diffraction using an X-ray wavelength of 1.54 Å.

28. The crystalline HBr salt form of claim 1, wherein the crystalline HBr salt form is characterized by a powder X-ray diffraction pattern comprising at least seven peaks selected from 7.5, 10.1, 11.8, 12.5, 12.8, 13.2, 13.6, 15.3, 16.0, 16.4, 17.4, 17.9, 18.8, 19.8, 20.3, 20.9, 21.3, 22.0, 22.9, 23.3, 23.8, 24.7, 25.2, 26.4, 27.8, 28.9, 30.0, 32.8, and 34.7±0.2 degrees 2 theta as measured by X-ray powder diffraction using an X-ray wavelength of 1.54 Å.

29. The crystalline HBr salt form of claim 1, wherein the crystalline HBr salt form is characterized by a powder X-ray diffraction pattern comprising peaks at 7.5, 10.1, 11.8, 12.5, 12.8, 13.2, 13.6, 15.3, 16.0, 16.4, 17.4, 17.9, 18.8, 19.8, 20.3, 20.9, 21.3, 22.0, 22.9, 23.3, 23.8, 24.7, 25.2, 26.4, 27.8, 28.9, 30.0, 32.8, and 34.7±0.2 degrees 2 theta as measured by X-ray powder diffraction using an X-ray wavelength of 1.54 Å.

30. The crystalline HBr salt form of claim 1, wherein the crystalline HBr salt form is characterized by the powder X-ray diffraction pattern substantially as shown in FIG. 11 as measured by X-ray powder diffraction using an X-ray wavelength of 1.54 Å.

31. The crystalline HBr salt form of claim 1, wherein the crystalline HBr salt form is characterized by a differential scanning calorimetry thermogram comprising an endotherm with an onset of about 201±1° C.

32. The crystalline HBr salt form of claim 1, wherein the crystalline HBr salt is characterized by a thermogravimetric analysis thermogram comprising a weight loss of about 0.35%±0.1% when heated from about 25° C. to about 150° C.

33. The crystalline HBr salt of claim 1, wherein the crystalline HBr salt form is characterized by ¹³C solid state NMR comprising at least three peaks selected from peaks at approximately 170, 166, 160, 159, 158, 157, 141, 137, 135, 132, 129, 126, 124, 117, 115, 111, 65, 60, 52, 46, 44, 30, 28, 24, and 19 ppm.

34. The crystalline HBr salt of claim 1, wherein the crystalline HBr salt form is characterized by ¹³C solid state NMR comprising at least five peaks selected from peaks at approximately 170, 166, 160, 159, 158, 157, 141, 137, 135, 132, 129, 126, 124, 117, 115, 111, 65, 60, 52, 46, 44, 30, 28, 24, and 19 ppm.

35. The crystalline HBr salt of claim 1, wherein the crystalline HBr salt form is characterized by ¹³C solid state NMR comprising at least seven peaks selected from peaks at approximately 170, 166, 160, 159, 158, 157, 141, 137, 135, 132, 129, 126, 124, 117, 115, 111, 65, 60, 52, 46, 44, 30, 28, 24, and 19 ppm.

36. The crystalline HBr salt of claim 1, wherein the crystalline HBr salt form is characterized by ¹³C solid state NMR comprising peaks at approximately 170, 166, 160, 159, 158, 157, 141, 137, 135, 132, 129, 126, 124, 117, 115, 111, 65, 60, 52, 46, 44, 30, 28, 24, and 19 ppm.

37. The crystalline HBr salt form of claim 1, wherein the crystalline HBr salt form is characterized by ¹³C solid state NMR substantially as depicted in FIG. 14.

38. The crystalline HBr salt form of claim 1, wherein the crystalline HBr salt form is substantially free of other crystalline or amorphous forms.

39. The crystalline HBr salt form of claim 38, wherein the amount of other crystalline or amorphous forms is 5% (w/w) or less.

40. A pharmaceutical composition comprising the crystalline salt form of Compound A of claim 1, and a pharmaceutically acceptable excipient.

41. A method of treating a disease mediated by C5aR in a subject in need of treatment, the method comprising administering to the subject a therapeutically effective amount of the crystalline salt form of Compound A of claim 1.

42. The method of claim 41, wherein the disease mediated by C5aR is anti-neutrophil cytoplasmic autoantibody-associated vasculitis, complement 3 glomerulopathy, hidradenitis suppurativa, or lupus nephritis, or any combination of the foregoing.

43. The method of claim 42, wherein the disease mediated by C5aR is anti-neutrophil cytoplasmic autoantibody-associated vasculitis.

44. The method of claim 41, wherein the compound is administered at a total daily dose of EQ 60 mg free base of Compound A or EQ 30 mg freebase of compound A twice daily.

45. The crystalline HCl salt form of claim 1, wherein the crystalline HCl salt form is a crystalline toluene solvate.

46. The crystalline toluene solvate of claim 45, wherein the crystalline toluene solvate is characterized by a powder X-ray diffraction pattern comprising at least three peaks selected from 6.3, 13.5, 16.7, 17.1, 18.9, 20.5, 23.4, and 24.1±0.2 degrees 2 theta as measured by X-ray powder diffraction using an X-ray wavelength of 1.54 Å.

47. The crystalline toluene solvate of claim 46, further characterized by one or more peaks selected from 5.1, 9.8, 11.9, 12.6, 14.4, 15.3, 17.8, 18.2, 19.5, 21.4, 22.6, and 24.9±0.2 degrees 2 theta as measured by X-ray powder diffraction using an X-ray wavelength of 1.54 Å.

48. The crystalline toluene solvate of claim 45, wherein the crystalline toluene solvate is characterized by a powder X-ray diffraction pattern comprising at least 3 peaks selected at 5.2, 11.9, 13.7, 14.9, 15.6, and 16.4±0.2 degrees 2 theta as measured by X-ray powder diffraction using an X-ray wavelength of 1.54 Å.

49. The crystalline toluene solvate of claim 48, further characterized by one or more peaks selected from 6.6, 8.1, 8.6, 10.3, 17.2, 18.0, 18.8, 21.6, 22.1, 22.8, 23.4, and 24.3, ±0.2 degrees 2 theta as measured by X-ray powder diffraction using an X-ray wavelength of 1.54 Å.

50. The crystalline HBr salt form of claim 1, wherein the crystalline HBr salt form is a crystalline toluene solvate.

51. The crystalline toluene solvate of claim 50, wherein the crystalline toluene solvate is characterized by a powder X-ray diffraction pattern comprising at least three peaks selected at 6.4, 8.4, 11.7, 12.9, 13.6, 15.3, 16.2, 21.8, and 24.0±0.2 degrees 2 theta as measured by X-ray powder diffraction using an X-ray wavelength of 1.54 Å.

52. The crystalline toluene solvate of claim 51, further characterized by one or more of the following:
(a) one or more peaks selected 10.1, 16.9, 17.6, 18.4, 19.2, 19.8, 20.4, 21.0, 21.4, 22.6, 23.0, and 24.9±0.2 degrees 2 theta as measured by X-ray powder diffraction using an X-ray wavelength of 1.54 Å;
(b) a differential scanning calorimetry thermogram comprising an endotherm with an onset of about 195±1° C.; or
(c) a thermogravimetric analysis thermogram comprising a weight loss of about 8%±1% when heated from about 25° C. to about 150° C.

53. An amorphous salt form of (2R,3S)-2-(4-(cyclopentylamino)phenyl)-1-(2-fluoro-6-methylbenzoyl)-N-(4-methyl-3-(trifluoromethyl)phenyl)piperidine-3-carboxamide (Compound A), wherein the amorphous salt form of Compound A is an amorphous HCl salt form or an amorphous HBr salt form.

54. The amorphous HCl salt form of claim 53 characterized by one or more of the following:
(a) a powder X-ray diffraction pattern substantially as shown in FIG. 1 using an X-ray wavelength of 1.54 Å;
(b) a modulated differential scanning calorimetry thermogram comprising a glass transition temperature of about 74±1° C.; or
(c) a thermogravimetric analysis thermogram comprising a weight loss of about 0.6%±0.1% when heated from about 25° C. to about 125° C.

55. The amorphous HCl salt form of claim 54, wherein the amorphous HCl salt form is substantially free of other crystalline or amorphous forms.

56. The amorphous HCl salt form of claim 54, wherein the amount of other crystalline or amorphous forms is 5% (w/w) or less.

57. The amorphous HBr salt form of claim 53 characterized by one or more of the following:
(a) a powder X-ray diffraction pattern substantially as shown in FIG. 4 using an X-ray wavelength of 1.54 Å;
(b) a modulated differential scanning calorimetry thermogram comprising a glass transition temperature of about 65±1° C.; or
(c) a thermogravimetric analysis thermogram comprising a weight loss of about 0.5%±0.1% when heated from about 25° C. to about 150° C.

58. The amorphous HBr salt form of claim 57, wherein the amorphous HBr salt form is substantially free of other crystalline or amorphous forms.

59. The amorphous HBr salt form of claim 58, wherein the amount of other crystalline or amorphous forms is 5% (w/w) or less.

60. A pharmaceutical composition comprising the amorphous salt form of Compound A of claim 51 and a pharmaceutically acceptable excipient.

61. A method of treating a disease mediated by C5aR in a subject in need of treatment, the method comprising administering to the subject a therapeutically effective amount of the amorphous salt form of Compound A of claim 53.

62. The method of claim 61, wherein the disease mediated by C5aR is anti-neutrophil cytoplasmic autoantibody-associated vasculitis, complement 3 glomerulopathy, hidradenitis suppurativa, or lupus nephritis, or any combination of the foregoing.

63. The method of claim 62, wherein the disease mediated by C5aR is anti-neutrophil cytoplasmic autoantibody-associated vasculitis.

64. The method of claim 1, wherein the compound is administered at a total daily dose of EQ 60 mg free base of Compound A or EQ 30 mg freebase of compound A twice daily.

65. The crystalline HCl salt form of claim 1, wherein the crystalline HCl salt form is characterized by a powder X-ray diffraction pattern comprising at least one peak selected from 3.9, 10.5, 12.3, 12.6, 13.9, 15.6, 18.1, 18.6, 21.5, and 25.2±0.2 degrees 2 theta as measured by X-ray powder diffraction using an X-ray wavelength of 1.54 Å.

66. The crystalline HCl salt form of claim 1, wherein the crystalline HCl salt form is characterized by a powder X-ray diffraction pattern comprising at least two peaks selected from 3.9, 10.5, 12.3, 12.6, 13.9, 15.6, 18.1, 18.6, 21.5, and 25.2±0.2 degrees 2 theta as measured by X-ray powder diffraction using an X-ray wavelength of 1.54 Å.

67. The crystalline HCl salt form of claim 1, wherein the crystalline HCl salt form is characterized by a powder X-ray diffraction pattern comprising at least three peaks selected from 3.9, 10.5, 12.3, 12.6, 13.9, 15.6, 18.1, 18.6, 21.5, and 25.2±0.2 degrees 2 theta as measured by X-ray powder diffraction using an X-ray wavelength of 1.54 Å.

68. The crystalline HCl salt form of claim 1, wherein the crystalline HCl salt form is characterized by a powder X-ray diffraction pattern comprising peaks at 3.9, 10.5, 12.3, 12.6, 13.9, 15.6, 18.1, 18.6, 21.5, and 25.2±0.2 degrees 2 theta as measured by X-ray powder diffraction using an X-ray wavelength of 1.54 Å.

69. The crystalline HCl salt form of claim 1, wherein the crystalline HCl salt form is characterized by a powder X-ray diffraction pattern comprising at least three peaks selected from 3.9, 5.2, 7.7, 10.5, 11.0, 11.7, 12.3, 12.6, 13.9, 14.6, 15.0, 15.6, 17.1, 18.1, 18.6, 19.6, 20.2, 21.5, 21.9, 22.9, 24.5, 25.2, 27.1, 27.9, 29.4, and 35.7±0.2 degrees 2 theta as measured by X-ray powder diffraction using an X-ray wavelength of 1.54 Å.

70. The crystalline HCl salt form of claim 1, wherein the crystalline HCl salt form is characterized by a powder X-ray diffraction pattern comprising at least five peaks selected from 3.9, 5.2, 7.7, 10.5, 11.0, 11.7, 12.3, 12.6, 13.9, 14.6, 15.0, 15.6, 17.1, 18.1, 18.6, 19.6, 20.2, 21.5, 21.9, 22.9, 24.5, 25.2, 27.1, 27.9, 29.4, and 35.7±0.2 degrees 2 theta as measured by X-ray powder diffraction using an X-ray wavelength of 1.54 Å.

71. The crystalline HCl salt form of claim 1, wherein the crystalline HCl salt form is characterized by a powder X-ray diffraction pattern comprising at least seven peaks selected from 3.9, 5.2, 7.7, 10.5, 11.0, 11.7, 12.3, 12.6, 13.9, 14.6, 15.0, 15.6, 17.1, 18.1, 18.6, 19.6, 20.2, 21.5, 21.9, 22.9, 24.5, 25.2, 27.1, 27.9, 29.4, and 35.7±0.2 degrees 2 theta as measured by X-ray powder diffraction using an X-ray wavelength of 1.54 Å.

72. The crystalline HCl salt form of claim 1, wherein the crystalline HCl salt form is characterized by a powder X-ray diffraction pattern comprising peaks at 3.9, 5.2, 7.7, 10.5, 11.0, 11.7, 12.3, 12.6, 13.9, 14.6, 15.0, 15.6, 17.1, 18.1, 18.6, 19.6, 20.2, 21.5, 21.9, 22.9, 24.5, 25.2, 27.1, 27.9, 29.4, and 35.7, and 28.3±0.2 degrees 2 theta as measured by X-ray powder diffraction using an X-ray wavelength of 1.54 Å.

73. The crystalline HCl salt form of claim 1, wherein the crystalline HCl salt form is characterized by the powder X-ray diffraction pattern substantially as shown in FIG. 28 as measured by X-ray powder diffraction using an X-ray wavelength of 1.54 Å.

74. The crystalline HCl salt form of claim 1, wherein the crystalline HCl salt form is characterized by a differential scanning calorimetry thermogram comprising an endotherm with an onset of about 192±1° C.

75. The crystalline HCl salt form of any one of claim 1, wherein the crystalline HCl salt is characterized by a thermogravimetric analysis thermogram comprising a weight loss of about 0.2%±0.1% when heated from about 25° C. to about 150° C.

76. The crystalline HCl salt form of any one of claim 1, wherein the crystalline HCl salt is characterized by a thermogravimetric analysis thermogram comprising a weight loss of about 0.2%±0.05% when heated from about 25° C. to about 150° C.

77. The crystalline HCl salt of any one of claim 1, wherein the crystalline HCl salt form is characterized by ¹³C solid state NMR comprising at least three peaks selected from peaks at approximately 170.2, 166.4, 158.9, 158.0, 157.3, 139.8, 138.1, 136.6, 134.9, 132.7, 131.6, 130.5, 128.4, 127.0, 126.5, 123.5, 122.4, 119.6, 114.9, 113.6, 65.0, 63.3, 56.4, 52.4, 47.7, 46.9, 42.9, 30.1, 25.8, 25.4, 22.9, and 19.3.

78. The crystalline HCl salt of claim 1, wherein the crystalline HCl salt form is characterized by ¹³C solid state NMR comprising at least five peaks selected from peaks at approximately 170.2, 166.4, 158.9, 158.0, 157.3, 139.8, 138.1, 136.6, 134.9, 132.7, 131.6, 130.5, 128.4, 127.0, 126.5, 123.5, 122.4, 119.6, 114.9, 113.6, 65.0, 63.3, 56.4, 52.4, 47.7, 46.9, 42.9, 30.1, 25.8, 25.4, 22.9, and 19.3.

79. The crystalline HCl salt of any one of claim 1, wherein the crystalline HCl salt form is characterized by ¹³C solid state NMR comprising at least seven peaks selected from peaks at approximately 170.2, 166.4, 158.9, 158.0, 157.3, 139.8, 138.1, 136.6, 134.9, 132.7, 131.6, 130.5, 128.4, 127.0, 126.5, 123.5, 122.4, 119.6, 114.9, 113.6, 65.0, 63.3, 56.4, 52.4, 47.7, 46.9, 42.9, 30.1, 25.8, 25.4, 22.9, and 19.3 ppm.

80. The crystalline HCl salt of any one of claim 1, wherein the crystalline HCl salt form is characterized by ¹³C solid state NMR comprising peaks at approximately 170.2, 166.4, 158.9, 158.0, 157.3, 139.8, 138.1, 136.6, 134.9, 132.7, 131.6, 130.5, 128.4, 127.0, 126.5, 123.5, 122.4, 119.6, 114.9, 113.6, 65.0, 63.3, 56.4, 52.4, 47.7, 46.9, 42.9, 30.1, 25.8, 25.4, 22.9, and 19.3 ppm.

81. The crystalline HCl salt form of claim 1, wherein the crystalline HCl salt form is substantially free of other crystalline or amorphous forms.

82. The crystalline HCl salt form of claim 1, wherein the crystalline HCl salt form is a THF/H₂O solvate.

83. The crystalline THF/H₂O solvate of claim 81, wherein the crystalline THF/H₂O solvate is characterized by a powder X-ray diffraction pattern comprising at least three peaks selected from 9.4, 13.4, 14.7, 15.7, 17.0, 17.8, 18.9, 19.5, 20.8, 22.6, 23.6, 25.0, 26.8, 27.2, 28.3, 29.0, 31.3, 34.5, 36.2, and 40.6±0.2 degrees 2 theta as measured by X-ray powder diffraction using an X-ray wavelength of 1.54 Å.

84. The crystalline THF/H₂O solvate of claim 83, further characterized by one or more of the following:
(d) one or more peaks selected from 9.4, 15.7, 17.0, 17.8, 20.8, 27.2, 28.3, 29.0, 31.3, 34.5, 36.2, and 40.6±0.2 degrees 2 theta as measured by X-ray powder diffraction using an X-ray wavelength of 1.54 Å;
(e) a differential scanning calorimetry thermogram comprising an endotherm with an onset of about 117±1° C.; or
(f) a thermogravimetric analysis thermogram comprising a weight loss of about 9%±1% when heated from about 25° C. to about 100° C.

85. The crystalline THF/H₂O solvate of claim 84, wherein the amount of other crystalline or amorphous forms is 5% (w/w) or less.

86. The crystalline HCl salt form of claim 3, wherein the crystalline HCl salt form is characterized by a powder X-ray diffraction pattern comprising peaks at 13.7, 15.4 and 16.3±0.2 degrees 2 theta as measured by X-ray powder diffraction using an X-ray wavelength of 1.54 Å.

87. The crystalline HBr salt form of claim 22, wherein the crystalline HBr salt form is characterized by a powder X-ray diffraction pattern comprising peaks at 7.5, 13.2, and 15.3±0.1 degrees 2 theta as measured by X-ray powder diffraction using an X-ray wavelength of 1.54 Å.
